SEQUENCE LISTING (33 sequences)

SEQ ID NO: 1 - DNA, 23 bp, synthetic construct
  source 1..23: mol_type = other DNA
cctgcactgaatcaagaggttgc

SEQ ID NO: 2 - DNA, 22 bp, synthetic construct
  source 1..22: mol_type = other DNA
ccatcagaaggacttgctggct

SEQ ID NO: 3 - DNA, 22 bp, synthetic construct
  source 1..22: mol_type = other DNA
gctaccagatggaaactgtgcg

SEQ ID NO: 4 - DNA, 23 bp, synthetic construct
  source 1..23: mol_type = other DNA
gcctctgtaatgttggtgagatc

SEQ ID NO: 5 - DNA, 23 bp, synthetic construct
  source 1..23: mol_type = other DNA
gcaactcgactatggcatctacg

SEQ ID NO: 6 - DNA, 22 bp, synthetic construct
  source 1..22: mol_type = other DNA
cggaatgaggtccacatcactg

SEQ ID NO: 7 - DNA, 23 bp, synthetic construct
  source 1..23: mol_type = other DNA
gcctctcattgtcaccgaagaac

SEQ ID NO: 8 - DNA, 22 bp, synthetic construct
  source 1..22: mol_type = other DNA
tggctgacgttggagatcacca

SEQ ID NO: 9 - DNA, 22 bp, synthetic construct
  source 1..22: mol_type = other DNA
gcagtgagcatcgaagagtacc

SEQ ID NO: 10 - DNA, 23 bp, synthetic construct
  source 1..23: mol_type = other DNA
ggcaaaagcatctggagttccag

SEQ ID NO: 11 - DNA, 22 bp, synthetic construct
  source 1..22: mol_type = other DNA
gaaggaacacggctgtcagcaa

SEQ ID NO: 12 - DNA, 22 bp, synthetic construct
  source 1..22: mol_type = other DNA
gatgagcaggttgctgttctgg

SEQ ID NO: 13 - DNA, 22 bp, synthetic construct
  source 1..22: mol_type = other DNA
gagcctactttgtacgtggcga

SEQ ID NO: 14 - DNA, 23 bp, synthetic construct
  source 1..23: mol_type = other DNA
gctctttccagactatgtaggag

SEQ ID NO: 15 - DNA, 22 bp, synthetic construct
  source 1..22: mol_type = other DNA
catcagtgtgcctgtaccagag

SEQ ID NO: 16 - DNA, 23 bp, synthetic construct
  source 1..23: mol_type = other DNA
gaggaatcacgtagcacttgtcc

SEQ ID NO: 17 - DNA, 22 bp, synthetic construct
  source 1..22: mol_type = other DNA
ctgctgtaacgatgaagccctg

SEQ ID NO: 18 - DNA, 22 bp, synthetic construct
  source 1..22: mol_type = other DNA
gctgtaggaagctcatctctcc

SEQ ID NO: 19 - DNA, 22 bp, synthetic construct
  source 1..22: mol_type = other DNA
tctgtgccagacacgattgacg

SEQ ID NO: 20 - DNA, 22 bp, synthetic construct
  source 1..22: mol_type = other DNA
gaggcagagttcagagccaagt

SEQ ID NO: 21 - DNA, 22 bp, synthetic construct
  source 1..22: mol_type = other DNA
cctctcatcacctacagcaacg

SEQ ID NO: 22 - DNA, 22 bp, synthetic construct
  source 1..22: mol_type = other DNA
ctggagacagtgggtaatacgg

SEQ ID NO: 23 - DNA, 20 bp, synthetic construct
  source 1..20: mol_type = other DNA
aggtgccctactacctggag

SEQ ID NO: 24 - DNA, 20 bp, synthetic construct
  source 1..20: mol_type = other DNA
gtctctctcggccattctgg

SEQ ID NO: 25 - DNA, 18 bp, synthetic construct
  source 1..18: mol_type = other DNA
catccatccatccatcca

SEQ ID NO: 26 - DNA, 20 bp, synthetic construct
  source 1..20: mol_type = other DNA
ttcagggtcatggagacagt

SEQ ID NO: 27 - DNA, 20 bp, synthetic construct
  source 1..20: mol_type = other DNA
aggtctacccgccatacctt

SEQ ID NO: 28 - DNA, 20 bp, synthetic construct
  source 1..20: mol_type = other DNA
cgccatagtgacagccagat

SEQ ID NO: 29 - DNA, 22 bp, synthetic construct
  source 1..22: mol_type = other DNA
agattcgggatatgctgttggc

SEQ ID NO: 30 - DNA, 21 bp, synthetic construct
  source 1..21: mol_type = other DNA
tcgggtcctagaccagtgttc

SEQ ID NO: 31 - RNA, 21 nt, synthetic construct
  source 1..21: mol_type = other RNA
atattcagaatagatcatggg

SEQ ID NO: 32 - RNA, 21 nt, synthetic construct
  source 1..21: mol_type = other RNA
tgtgcttcaacattctccctc

SEQ ID NO: 33 - RNA, 21 nt, synthetic construct
  source 1..21: mol_type = other RNA
tgcttaatcacaagagggctt

(19) United States
(12) Patent Application Publication
(10) Pub. No.: US 2024/0398831 A1
(43) Pub. Date: Dec. 5, 2024

(54) COMPOSITIONS COMPRISING SMALL MOLECULE REGULATORS OF TUMOR IMMUNITY AND METHODS OF USING SAME

(71) Applicant: Duke University, Durham, NC (US)

(72) Inventors: Donald McDonnell, Durham, NC (US); Binita Chakraborty, Durham, NC (US); Ching-Yi Chang, Durham, NC (US); Jovita Byemerwa, Durham, NC (US)

(21) Appl. No.: 18/697,976

(22) PCT Filed: Oct. 5, 2022
(86) PCT No.: PCT/US2022/045822

§ 371 Date: Apr. 2, 2024

Related U.S. Application Data
(60) Provisional application No. 63/252,298, filed on Oct. 5, 2021.

(51) Int. Cl. A61K 31/565; A61K 39/00; A61K 39/395; A61K 45/06; A61P 35/00; C12Q 1/6886

(52) U.S. Cl. CPC A61K 31/565; A61K 39/39558; A61K 45/06; A61P 35/00; C12Q 1/6886; A61K 2039/507; C12Q 2600/106; C12Q 2600/158

(57) ABSTRACT

Disclosed herein are compositions and methods for treating cancer. The methods may include administering to a subject at least one estrogen receptor (ER) modulating drug. The methods may further include administering to the subject at least one additional therapy. Further provided herein are methods of predicting response of a subject to immune checkpoint blockade (ICB) therapy.

### CROSS-REFERENCE TO RELATED APPLICATIONS

This application claims priority to U.S. Provisional Patent Application No. 63/252,298, filed Oct. 5, 2021, the entire contents of which are hereby incorporated by reference.

### STATEMENT REGARDING FEDERALLY SPONSORED RESEARCH

This invention was made with government support under grant BC170954 awarded by the United States Department of Defense. The government has certain rights in the invention.

### FIELD

This disclosure relates to compositions and methods for treating cancer, including estrogen receptor (ER) modulating drugs.

### INTRODUCTION

Metastatic melanoma is one of the most aggressive, morbid cancers with a median survival of 6-9 months. Whereas the development of MAPK-pathway inhibitors and antibodies directed against immune checkpoints have significantly improved outcome in this disease, de novo and acquired resistance to these therapies remains a major impediment to achieving durable clinical responses in most patients. Further, although complete responses to combination immune checkpoint blockade (ICB) therapies (for example, α-CTLA4, α-PD1) occurs in ˜20% of patients, the general toxicity and immune related adverse events seen in the majority of individuals receiving existing combination therapies significantly limits their clinical use. Thus, strategies that increase the efficacy and/or reduce the toxicities associated with ICB would likely expand the clinical utility of existing drugs and ultimately improve long-term outcomes in this disease.

The classification of melanoma as a hormone-sensitive neoplasm remains controversial and the importance of hormone associated risk factors, such as pregnancy, menopausal status, hormone therapies, and the use of oral contraceptives, on the pathobiology of this disease remains unclear. While the potential effects of sex steroids on melanoma risk needs to be assessed in large clinical studies, there already exists compelling evidence that the incidence of secondary melanoma is significantly lower in anti-estrogen treated breast cancer patients than in the general population. Further, the results of a recently published meta-analysis revealed that the degree of benefit from ICB in melanoma, and in patients with non-small cell lung cancer, is lower in women than in men.

Under normal physiological conditions and in some disease contexts, it has been demonstrated that female sex steroids that target the estrogen receptor (ER) affect the differentiation and function of both the humoral and adaptive immune systems. However, it has not been established whether the extent to which estrogen action/signaling in the tumor-immune microenvironment impacts the growth of melanoma or if and how this signaling axis can be exploited for therapeutic benefit.

Estrogens mediate their physiological actions in cells through the classical nuclear ERs (ERα and ERβ) and through the non-classical G-protein coupled receptor GPER1 (also referred to as GPR30). A recent study highlighted a tumor cell-intrinsic role for GPER1 in regulating melanocyte differentiation, thereby preventing melanoma cell proliferation. Further, a synergistic anti-tumor response was observed when GPER agonists were combined with immune checkpoint inhibitors. While anecdotal evidence exists regarding the expression of nuclear ERs in melanoma cancer cells, the extent to which these receptors play a role in tumor progression remains to be determined. ERs have also been shown to be expressed in several different cell types within the tumor microenvironment and may play a role in determining tumor response to ER modulators. Indeed, 17β-estradiol (E2) working through ERα expressed in endothelial cells in the tumor microenvironment has been shown to induce tumor growth by improving tumor angiogenesis and protecting tumor cells against hypoxia and necrosis. Further. ER actions have been studied in different immune cell types in different diseases, but the extent to which ER influences immune cell biology within the tumor microenvironment has not been examined in detail. Recently, it has been demonstrated in ovarian cancer that E2 can create an immune suppressive tumor microenvironment (TME) by promoting the mobilization of myeloid-derived suppressor cells (MDSC) from bone, which function to suppress tumor immunity and increase tumor growth. While this study demonstrates that ER function is important for MDSC mobilization, the tumor microenvironment is infiltrated with multiple other myeloid cell types such as dendritic cells (DCs), monocytes, and tumor associated macrophages, all of which impact tumor immunity. Notably. ERs have been shown to play a critical role in development and functionality of these myeloid cell types. However, the extent to which ER function regulates myeloid cell-T cell crosstalk within the TME and how it affects ICB responses are not known.

### SUMMARY

In an aspect, the disclosure relates to method of treating cancer in a subject. The method may include administering to the subject at least one estrogen receptor (ER) modulating drug and at least one additional therapy.

In a further aspect, the disclosure relates to method of treating cancer in a subject. The method may include administering to the subject at least one estrogen receptor (ER) modulating drug such that the effectiveness of an ICB therapy is increased relative to a control. In some embodiments, the method further includes administering to the subject the ICB therapy. In some embodiments, the ICB therapy is selected from anti-PD1, anti-CTLA4, anti-PDL1, and DMXAA, or a combination thereof. In some embodiments, the effectiveness of the ICB therapy is increased by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. In some embodiments, the method further includes administering to the subject at least one additional therapy.

In some embodiments, the at least one ER modulating drug is selected from a selective estrogen receptor modulator (SERM), a selective estrogen receptor degrader (SERD), an antiprogestin, an aromatase inhibitor, or a combination thereof. In some embodiments, the SERM is selected from lasofoxifene, bazodoxifene, tamoxifen, raloxifene, clomiphene, ospemiphene, arzoxifene, toremifene, and H3B6545, or a combination thereof. In some embodiments, the SERD is selected from fulvestrant, LSZ102, LY3484356, giredestrant, camizestrant, GDC0927, D-052, AC0682, AZD9496, SAR439859, RAD1901, G1T48, Zn-c5, ARV-471, and OP-1250, or a combination thereof. In some embodiments, the antiprogestin is selected from mifepristone, asoprisnil, onapristone, and telapristone, or a combination thereof. In some embodiments, the aromatase inhibitor is selected from letrozole, anastrozole, exemestane, vorozole, formestane, fadrozole, testolactone, aminoglutethimide, androstatrienedione, and 6-Oxo, or a combination thereof. In some embodiments, the at least one additional therapy is selected from chemotherapy, immunotherapy, radiation therapy, hormone therapy, targeted drug therapy, cryoablation, and surgery, or a combination thereof. In some embodiments, the chemotherapy is selected from an antimitotic agent, an alkylating agent, an antimetabolite, an antimicrotubule agent, a topoisomerase inhibitor, a cytotoxic agent, a cell cycle inhibitor, a growth factor inhibitor, a histone deacetylase (HDAC) inhibitor, and an inhibitor of a pathway that cross-talks with and activates ER transcriptional activity, or a combination thereof. In some embodiments, the alkylating agent is selected from cisplatin, oxaliplatin, chlorambucil, procarbazine, and carmustine, or a combination thereof. In some embodiments, the antimetabolite is selected from methotrexate, 5-fluorouracil, cytarabine, and gemcitabine, or a combination thereof. In some embodiments, the antimicrotubule agent is selected from vinblastine and paclitaxel, or a combination thereof. In some embodiments, the topoisomerase inhibitor is selected from etoposide and doxorubicin, or a combination thereof. In some embodiments, the cytotoxic agent comprises bleomycin. In some embodiments, the cell cycle inhibitor is selected from a cyclin-dependent kinase 4/6 (CDK4/6) inhibitor selected from palbociclib, abemaciclib, and ribociclib, or a combination thereof. In some embodiments, the growth factor inhibitor is selected from a human epidermal growth factor receptor 2 (HER2) inhibitor such as trastuzumab, deruxtecan, sacitizumab, or ado-trastuzumab emtansine. In some embodiments, the HDAC inhibitor is selected from vorinostat, romidepsin, chidamide, panobinostat, belinostat, Vvlproic acid, mocetinostat, abexinostat, entinostat, pracinostat, resminostat, givinostat, quisinostat, kevetrin, CUDC-101, AR-42, tefinostat, CHR-3996, 4SC202, CG200745, rocilinostat, and sulforaphane, or a combination thereof. In some embodiments, the entinostat is not administered with an HER2 inhibitor. In some embodiments, the inhibitor of a pathway that cross-talks with and activates ER transcriptional activity is selected from a phosphoinositide 3-kinase (PI3K) inhibitor, a heat shock protein 90 (HSP90) inhibitor, and a mammalian target of rapamycin (mTOR) inhibitor such as Everolimus. In some embodiments, the immunotherapy is selected from a checkpoint inhibitor and denosumab, or a combination thereof. In some embodiments, the checkpoint inhibitor is selected from anti-PD1, anti-CTLA4, anti-PDL1, and DMXAA, or a combination thereof. In some embodiments, the targeted drug therapy is selected from vemurafenib, anti-EGFR targeted therapy, a serotonin-norepinephrine reuptake inhibitor (SNRI), a selective serotonin reuptake inhibitor (SSRI), and gabapentin, or a combination thereof. In some embodiments, the at least one ER modulating drug is administered with anti-PD1, or with anti-CTLA4, or with anti-PD1 and anti-CTLA4. In some embodiments, the method further comprises administering vemurafenib. In some embodiments, the at least one ER modulating drug and the at least one additional therapy are administered simultaneously or sequentially. In some embodiments, the at least one ER modulating drug and the at least one additional therapy and the vemurafenib are administered simultaneously or sequentially. In some embodiments, the at least one ER modulating drug is administered to the subject once every day, once every 2 days, once every 3 days, once every 4 days, once every 5 days, once every 6 days, once every 7 days, once every week, once every 2 weeks, once every 3 weeks, once every 4 weeks, once every 5 weeks, once every 6 weeks, once every 7 weeks, once every 8 weeks, once every month, once every 2 months, once every 3 months, once every 4 months, once every 5 months, or once every 6 months. In some embodiments, the at least one ER modulating drug is administered to the subject for 1 year, 2 years, 3 years, 4 years, 5 years, or more than 5 years. In some embodiments, the at least one ER modulating drug is administered to the subject orally, intravenously, transdermally, or vaginally. In some embodiments, the ER is ER-alpha or ER-beta. In some embodiments, the cancer is selected from melanoma, colon cancer, breast cancer, and lung cancer. In some embodiments, tumor-associated macrophage (TAM) polarization towards an immune suppressive phenotype is reduced, or ER-alpha in myeloid cells is depleted, or the Wnt 5A/TCF4 pathway is reduced, or CD4+ T cell infiltration is not affected, or an interferon pathway is reduced, or CD8+ T cell proliferation is increased, or CD8+ T cell migration is increased, or CD8+ T cell cytotoxicity is increased, or the ratio of M1/M2 macrophages is increased, or tumor growth is decreased, or tumor size is decreased, or metastasis is reduced, or a combination thereof, in the subject.

Another aspect of the disclosure provides a composition for treating cancer. The composition may include at least one estrogen receptor (ER) modulating drug and at least one additional therapy. In some embodiments, the at least one ICB therapy is selected from anti-PD1, anti-CTLA4, anti-PDL1, and DMXAA, or a combination thereof.

Another aspect of the disclosure provides a method of predicting response of a subject to ICB therapy. The method may include determining the level of expression in the subject of a gene selected from “Genes up-regulated upon E2 treatment” in TABLE 5 and/or “Genes down-regulated upon E2 treatment” in TABLE 5. The level of expression of the gene selected from “Genes down-regulated upon E2 treatment” may be increased relative to a control, and/or the level of expression of the gene selected from “Genes up-regulated upon E2 treatment” is decreased relative to a control. The method may further include thereby identifying the subject as responsive to ICB therapy. In some embodiments, the method further includes administering to the subject at least one ICB therapy. In some embodiments, the at least one ICB therapy is selected from anti-PD1, anti-CTLA4, anti-PDL1, and DMXAA, or a combination thereof.

The disclosure provides for other aspects and embodiments that will be apparent in light of the following detailed description and accompanying figures.

### BRIEF DESCRIPTION OF THE DRAWINGS

FIGS. 1A-1E show that intratumoral MDSC do not predict ICB responses in melanoma patients. The relative proportion of MDSC/neutrophils was determined by applying published MDSC signatures from (FIG. 1A) PMID: 21954284, (FIG. 1B) PMID: 23152559, (FIG. 1C) PMID. 28052254, (FIG. 1D) PMID.25822800, and (FIG. 1E) PMID.24138885, and was then applied to analyze data from melanoma patients parsed by their response to pembrolizumab/nivolumab treatment alone. CR=complete response; PR=partial response; SD=stable disease; and PD=progressive disease, obtained from a previous dataset (Gide et al., Cancer Cell. 2019, 35, 238-255, incorporated herein by reference).

FIGS. 2A-2G show that a decreased M1/M2 ratio compromises benefit to immunotherapy in melanoma patients. FIGS. 2A-2B show the relative proportion of M1 macrophages as determined by CIBERSORT or the ratio of M1/M2 macrophages in melanoma patients parsed by their response to immunotherapies in the same patient cohort. FIGS. 2C-2D show the median overall survival in all patient cohorts (Gide et al., Cancer Cell. 2019, 35, 238-255, incorporated herein by reference) treated with immunotherapy with either high or low proportions of M1 macrophages or M1/M2 ratio as determined by CIBERSORT. FIG. 2E shows the median overall survival in all patient cohorts treated with Ipilimumab alone (Van Allen et al., Science. 2015, 350, 207-211, incorporated herein by reference) or either Pembrolizumab or Nivolumab alone (Hugo et al., Cell. 2016, 165, 35-44, incorporated herein by reference), with either a high or low M1/M2 signature ratio as determined by CIBERSORT. FIGS. 2F-2G show CD68, CSF1, CSF1R, and PDCD1 expression in melanoma patients who were classified as non-responders (n=13) and responders (n=12) to anti-PD1 therapy, obtained from previous datasets (Hugo et al., Cell. 2016, 165, 35-44). Both responders and non-responders were stratified to CYP19A1^{hi} and CYP19A1^{lo} by median expression. Significance was calculated using a paired t test (FIG. 2A, FIG. 2C, and FIGS. 2D-2E) and by log rank test (FIGS. 2B and 2F-2G).

FIGS. 3A-3N show that a decreased M1/M2 ratio compromises benefit to immunotherapy in melanoma patients. FIG. 3A shows the relative proportion of M2 macrophages or the ratio of M1/M2 macrophages as determined by CIBERSORT in melanoma patients parsed by their response to pembrolizumab/nivolumab treatment alone. CR=complete response; PR=partial response; SD=stable disease; and PD=progressive disease, obtained from a previous dataset (Gide et al., Cancer Cell. 2019, 35, 238-255, incorporated herein by reference). FIG. 3B shows the median overall survival in all patient cohorts (Gide et al., Cancer Cell. 2019, 35, 238-255, incorporated herein by reference) treated with immunotherapy with either high (upper 50%) or low (lower 50%) proportions of M2 macrophages as determined by CIBERSORT. FIGS. 3C-3E show the relative proportion of M1 and M2 macrophages or the ratio of M1/M2 macrophages as determined by CIBERSORT in melanoma patients parsed by their response to pembrolizumab/nivolumab treatment alone. FIGS. 3F-3H show the median overall survival in all patient cohorts treated with anti-P1 monotherapy (pembrolizumab or nivolumab) with either high (upper 50%) or low (lower 50%) relative proportion of M1 or M2 macrophages or M1/M2 macrophage ratio as determined by CIBERSORT. FIGS. 31-3K show the relative proportion of M1 and M2 macrophages or the ratio of M1/M2 macrophages in melanoma patients parsed by their response to Ipilimumab+Nivolumab and Ipilimumab+Nivolumab combination therapies. CR=complete response; PR=partial response; SD=stable disease; and PD=progressive disease, obtained from a previous dataset (Gide et al., Cancer Cell. 2019, 35, 238-255, incorporated herein by reference). FIGS. 3L-3N show the median overall survival in all patient cohorts treated with combination immunotherapy (Ipilimumab+Nivolumab and Ipilimumab+Nivolumab) with either high or low proportions M1 or M2 macrophages or M1/M2 macrophage ratio.

FIGS. 4A-4C show that intratumoral M1/M2 macrophages dictate survival outcomes in melanoma patients. FIGS. 4A-4C show the median overall survival of all melanoma patients (FIG. 4A), treated patients (FIG. 4B), or untreated patients (FIG. 4C) with either a high (upper 50%) or low (lower 50%) M1/M2 macrophage ratio as obtained from TCGA SKCM dataset.

FIGS. 5A-5F show that melanoma tumor cells do not respond to E2 in vitro. FIG. 5A shows an immunoblot of ERα in melanoma cell lines B16F10 and YuMM5.2. ERα+ MCF7 cells served as a positive control. Cells were treated as either mock transfected or transfected with scrambled control or three different siRNA targeting Esr1. FIG. 5B shows quantitative PCR analysis of Esr1 gene expression in B16F10 and YuMM5.2 cell lines from experiments described in FIG. 5A. Esr1 was first normalized to Rplp0 and then Esr1 knockdown samples were normalized to a non-targeting control. RNA was collected from both cell lines after 48 hr of siRNA transfection. FIG. 5C shows quantitative PCR of Esr1 and ER target genes Pgr and Cxc112 in YuMM5.2 and B16F10 cells treated with either DMSO, E2 (1 nM), or E2 (1 nM)+fulvestrant (100 nM) for 16 hr. Mouse ovary served as a positive control. Individual targets were normalized to Rplp0 and then treated samples (E2 and fulvestrant) and ovary samples were normalized to DMSO. FIGS. 5D-5E show proliferation of B16F10 cells over 3 days when treated with either vehicle DMSO or E2 (0.01-1 nM) (FIG. 5E, right) or E2 (0.01-1 nM)+fulvestrant (100 nM). ERα+MCF7 cells served as a positive control (FIG. 5E, left). FIG. 5F shows uterine wet weights from mice that were ovariectomized and supplemented with placebo or estrogen pellets. Data are expressed as individual data points and represented as mean±S.E.M. Significance was calculated by Student's t test or two-way ANOVA (FIGS. 5E-5F), followed by Bonferroni's multiple correction (**p<0.0001).

FIGS. 6A-6I show that E2 promotes melanoma tumor growth. FIGS. 6A-6B and FIG. 6E show subcutaneous tumor growth of B16F10 (1×10⁵ cells, n=10), Yumm5.2 (0.5×10⁵ cells, n=8), or BPD6 (0.5×10⁵ cells, n=5) cells in syngeneic C57BL/6J ovariectomized hosts supplemented with placebo or E2. FIG. 6C shows weights of YuMM5.2 tumors, resulting from experiments in FIG. 2B. FIG. 6D shows survival of mice harboring YuMM5.2 tumors, resulting from experiment in FIG. 2B. FIG. 6F shows tumor growth in iBP female mice that were ovariectomized and supplemented with either placebo or E2 pellets (n=5). Tumor formation in these mice were induced with a single intradermal dose of 150 μg of 4-hydroxytamoxifen (4OHT). FIGS. 6G-6H show survival and weights of tumors (Placebo vs. E2, n=6), resulting from experiments in FIG. 2F. FIG. 6I shows B16F10 (1×10⁵ cells, n=10) tumor growth in ovariectomized NSG (NOD.Cg⁻Prkd^{scid} Il2rg^{tm1Wjl}/SzJ) mice supplemented with placebo or E2. FIGS. 6A-6B and FIGS. 6E-6F are representative of two independent experiments. Data are expressed as mean t S.E.M. Significance was calculated using the Student's t test (FIG. 6C and FIG. 6H), log-rank test (FIG. 6D and FIG. 6G), and two-way ANOVA followed by Bonferroni's multiple correction (FIGS. 6A-6B, FIGS. 6E-6F, and FIG. 6I) (*p<0.05, *p<0.01, and ***<0.001).

FIGS. 7A-7Q show that E2 regulates myeloid cell function in the tumor microenvironment. FIGS. 7A-7B show uniform manifold approximation and projection (uMAP) plots of expression profiles for tumor infiltrating immune cells (CD45⁺) (n=3 tumors/each treatment, pooled together) isolated from iBP tumors. Each dot represents an individual cell (FIG. 7A). FIG. 7B shows the percentage of CD68⁺ macrophages/monocytes among all sequenced cell types determined by scRNA seq in placebo vs E2 treated samples. FIGS. 7C-7D show syngeneic tumor growth of B16F10 (1×10⁵) cells and YuMM5.2 (5×10⁵) cells in myeloid ERα knockout (Esr1^{f/f}; LysMCre) and littermate control (Esr1M and LysMCre) mice that were ovariectomized and supplemented with either placebo or E2 pellets. Esr1^{f/f}+Placebo (blue, n=10); LysMCre+Placebo (brown, n=7); Esr1^{f/f}; LysMCre+Placebo (black, n=8); Esr1^{f/f}+E2 (maroon, n=8); LysMCre+E2 (red, n=7); Esr1^{f/f}; LysMCre+E2 (purple, n=8). FIG. 7E shows tumor growth of YuMM5.2 cells (5×10⁵) in CD8⁺ T cell depleted C57BL6/J hosts that were ovariectomized and supplemented with placebo and E2 (n=8 mice per treatment). FIGS. 7F-7I show T cell proliferation that was assessed after co-culturing with tumor infiltrating CD11b⁺ cells isolated from iBP mice treated with either placebo or E2. Shown are representative CFSE dilution plots of CD8⁺ (FIG. 7F) and CD4⁺ (FIG. 7H) cells. Quantification of CFSE low/negative CD8⁺ (FIG. 7G) and CD4⁺ (FIG. 7I) populations is shown and expressed as percentage of CD8⁺ and CD4⁺ T cells (n=3), representative of two independent experiments. FIGS. 7J-7Q show representative flow cytometry plots and percentage of IFNγ⁺ and GZMB⁺ CD8⁺ T (FIGS. 7J-7M) or CD4⁺ T cells (FIGS. 7N-7Q) after 72 hr of co-culture with tumor infiltrating CD11b myeloid cells isolated from iBP mice treated with either placebo or E2, n=3 per group. Data are represented as mean±S.E.M. Significance was calculated using a Student's t test (FIG. 7G, FIG. 7I, FIG. 7K, FIG. 7M, FIG. 7O, and FIG. 7Q) and by two-way ANOVA (FIGS. 7C-7E), followed by Bonferroni's multiple correction (*p<0.05, **p<0.01, and **p<0.001).

FIGS. 8A-8D show the identification of immune cell types from scRNA seq. FIG. 8A shows uniform manifold approximation and projection (UMAP) plots of expression of marker genes that define each cluster Ptprc (all immune cells), Cd3e (all T cells), Cd68 (monocytes and macrophages), Kirb1c (NK cells). Kit (Mast Cells), Cd24 (DC), Cd19 (B cells), and Ly6g (granulocytes). FIGS. 8B-8D show UMAP plots of Esr1 (FIG. 8B), Esr2 (FIG. 8C), and Gper (FIG. 8D) in tumor infiltrating immune cells (CD45*) isolated from placebo and E2 treated tumors.

FIGS. 9A-9G show that E2 affects myeloid cell function. FIG. 9A shows the percentage of different immune cell types determined by scRNA seq in placebo and E2 treated melanoma tumors. FIG. 9B shows a violin plot demonstrating the top 20 up- and down-regulated genes in CD68⁺ cluster as determined by scRNA sequencing. FIG. 9C shows quantitative real time PCR of Esr1 expression in bone marrow derived macrophages isolated from Esr1^{f/f} (black, n=5) and Esr1^{f/f}; LysMCre (red, n=5) mice (left). Esr1 was normalized to the internal house-keeping control Rplp0 and then Esr1^{f/f}; LysMCre and ovary samples were normalized to Esr1^{f/f} samples. Absolute quantification of Esr1 was performed from the same experiment. Mouse ovary was used as a positive control (right). FIG. 9D shows weights of tumors resulting from implanting YuMM5.2 cells in Esr1^{f/f} (n=5) and Esr1^{f/f}; LysMCre (n=5) mice in the presence or absence of E2. FIG. 9E shows the percentage of F480⁺CD206⁻MHCIIhi macrophages in tumor infiltrating immune cells isolated from YuMM5.2 tumors implanted in Esr1^{f/f}+placebo, Esr1^{f/f}; LysMCre+placebo, Esr1^{f/f}+E2 and Esr1^{f/f}; LysMCre+E2 mice. Data are expressed as individual data points and represented as mean±S.E.M. Significance was calculated by one-way ANOVA followed by Bonferroni's multiple correction. FIGS. 9F-9G show quantification of CD8⁺ and CD4⁺ T cells derived from peripheral blood of mice used in experiment FIG. 6E (n=5 mice per group). Data are represented as mean±S.E.M. Significance was calculated by Student's t test (FIG. 9B) and one-way ANOVA (FIGS. 9C-9F) followed by Bonferronni's multiple correction (*p<0.05, *p<0.01, and ***p<0.001).

FIGS. 10A-10E show that E2 regulates TAM function in the melanoma tumor microenvironment. FIG. 10A shows the gating strategy for tumor infiltrating myeloid cells. FIGS. 10B-10C show the percentage of CD45⁺ tumor infiltrating immune cells isolated from iBP (n=3) and B16F10 (n=4-6) syngeneic tumors treated with placebo or E2. FIG. 10D shows quantification of tumor infiltrating MDSCs (Ly6C⁺ Ly6G⁺) in B16F10, YuMM5.2, and BPD6 tumors. FIG. 10E shows quantification of intratumoral macrophages upon clodronate-mediated depletion of myeloid cells. Data are expressed as individual data points and are represented as mean±S.E.M. Significance was calculated by one-way ANOVA followed by Bonferroni's multiple correction (*p<0.05).

FIGS. 11A-11Q show that E2 regulates TAM function. FIGS. 11A-11C show the ratio of M1 to M2 macrophages in iBP (n=6) (FIG. 11A) and B16F10 (n=6-10) (FIG. 11B) tumors from placebo and E2 treated mice and representative flow cytometry plots of M2 and M1 macrophages in the B16F10 model (FIG. 11C). FIG. 11D shows the growth of B16F10 tumors (n=12) upon depletion of macrophages by clodronate liposomes in ovariectomized mice supplemented with placebo or E2. FIGS. 11E-11F show the quantification of IFNγ⁺CD8⁺ T (FIG. 11E) and GZMB⁺CD8⁺ T (FIG. 11F) cells (n=3) that were cocultured with BMDM differentiated in NM or TCM and treated with either DMSO or E2 (1 nM). FIGS. 11G-11H show CFSE dilution and quantification representing proliferation of CFSE^{low/-} CD8⁺ T (n=3) after co-culturing with BMDM cells from Esr1^{f/f} and Esr1^{f/f}; LysMCre mice, differentiated in either normal media or TCM (B16F10), and followed by treatment with either DMSO or E2 (1 nM). FIGS. 11I-11K show quantification of IFNγ+, CD44⁺CD69⁺, and GZMB⁺ CD8 T cells (n=3) from the same experiment as in FIG. 11G. FIG. 11L shows the tumor co-mixing methodology. FIG. 11M shows the syngeneic tumor growth of YuMM5.2 (5×10⁵) cells co-mixed with BMDM from either (Esr1^{f/f}; LysMCre) or its littermate controls (Esr1^{f/f}) (1:1) in ovariectomized mice supplemented with either placebo or E2. (Esr1^{f/f} BMDM+YuMM5.2)—placebo (black solid circle, n=10), (Esr1^{f/f}; LysMCre, BMDM+YuMM5.2)—placebo (open square, n=10), (Esr1^{f/f} BMDM+YuMM5.2)—E2 (up triangle, n=10), and (Esr1^{f/f}; LysMCre, BMDM+YuMM5.2)—E2 (down triangle, n=10). FIG. 11N shows a UMAP representation of macrophage/monocyte subclusters as determined from scRNA sequencing. FIG. 11O shows trajectory analysis depicting the differentiation of monocytes into different lineages of macrophages. FIG. 11P shows the density of cells in macrophage/monocyte subclusters along a pseudotime gradient. FIG. 11Q shows the expression of M2 associated genes (Cd163, Lgr2, Retnia, and Folr2) in macrophage clusters along the pseudotime axis. FIGS. 11E-11F and FIGS. 11G-11K are representative of two independent experiments. Data are expressed as individual data points and represented by mean±S.E.M. Significance was calculated by Student's t test (FIGS. 11A-11B), one-way ANOVA (FIGS. 11E-11G and FIGS. 11I-11K), and two-way ANOVA (FIG. 11D and FIG. 11M) followed by Bonferroni's multiple correction (*p<0.05, **p<0.01 and ***p<0.001).

FIGS. 12A-12D show that depletion of ERα in myeloid cells suppresses melanoma tumor growth. FIG. 12A shows the percentage of F480⁺ macrophages from in vitro differentiated BMDM of genotypes Esr1^{f/f} and Esr1^{f/f}; LysMCre used for macrophage tumor cell co-mixing experiments. FIG. 12B shows the syngeneic tumor growth of B16F10 cells when co-mixed with BMDM from (Esr1^{f/f}; LysMCre) and littermate control (Esr1^{f/f}) mice. Mice of both genotypes were ovariectomized and supplemented with E2 pellets and injected with B16F10 (1×10⁵) cells+BMDM at a 1:1 ratio. (Esr1^{f/f} BMDM+B16F10)—E2 (open circle, n=10) and (Esr1^{f/f}; LysMCre, BMDM+B16F10)—E2 (black square, n=10). FIG. 12C shows violin plots of monocyte/macrophage genes from placebo and E2 treated tumors as determined by scRNA seq. FIG. 12D shows the distribution of different clusters along the pseudotime axis. Data are expressed as mean±S.E.M. Significance was calculated by Student's t test (FIG. 12A) and two-way ANOVA (FIG. 12B), followed by Bonferroni's multiple correction (*p<0.05, *p<0.01, **p<0.001, and **p<0.0001).

FIGS. 13A-13S show the identification of monocyte/macrophage subclusters from CD68⁺ cells. FIGS. 13A-13P show the expression of marker genes in different monocyte/macrophage subclusters. FIG. 13O shows a comparison of the proportion of cells from each cluster shown in FIG. 11N in tumors from placebo (black) and E2 (red) treated mice. FIGS. 13R-13S show the percentage of intratumoral (Ly6C⁺/Ly6G⁻) monocytes and F480⁺ macrophages as determined by flow cytometry from iBP tumors treated with placebo or E2.

FIGS. 14A-14F show that E2 regulates Wht5A/TCF4 pathways in myeloid cells. FIGS. 14A-14B show bubble plots showing the upregulation of TCF4 and WNT5A signaling in tumor associated macrophages as determined by upstream regulator analysis of DEGs in the macrophage cluster by ingenuity pathway analysis (IPA) software (red bubbles=upregulated; green bubbles=downregulated; solid line=direct target; dashed line=indirect target). FIG. 14C shows quantitative real time PCR of WNTSA and TCF4 target genes in tumor infiltrating myeloid cells isolated from iBP tumors treated with placebo and E2 (n=6 per group). FIG. 14D shows quantitative real time PCR of genes associated with angiogenesis from CD11b⁺ tumor infiltrating myeloid cells isolated from iBP tumors treated with placebo (black solid circle) and E2 (open circle) (n=3 per group). All target genes were normalized to Rplp0 and then E2 samples were normalized to placebo. FIGS. 14E-14F show quantitative real time PCR of genes associated with Wnt5A-S catenin targets in YuMM5.2 melanoma tumor cells depleted for Esr1 with siRNA or treated with DMSO, E2 (1 nM), or E2 (1 nM)+fulvestrant (100 nM). All target genes were normalized to Rplp0 and then siEsr1 (FIG. 14E) or DMSO (FIG. 14F) samples were normalized to siRNA control. Data are represented as mean±S.E.M. Significance was calculated by Student's t test (FIGS. 14C-14D) (*p<0.05, **p<0.01, and ***p<0.001).

FIGS. 15A-15L show that E2 suppresses the anti-tumor T cell response. Representative flow cytometry plots and quantification are shown of CD3⁺ (FIGS. 15A-15B) and CD8⁺ (FIGS. 15C-15D) tumor infiltrating lymphocytes in iBP (n=5-6) tumors isolated from mice treated with either placebo (black) or E2 (gray). FIGS. 15E-15L show representative flow cytometry plots and quantification of PD1⁺ (FIGS. 15E-15F), GZMB⁺ (FIGS. 15G-15H), CD44-CD69⁺ (FIGS. 15I-15J), and IFNγ+ (FIGS. 15K-15L) CD8⁺ T cells in YuMM5.2 tumors from mice treated with placebo (black solid circle) or E2 (open circle) (n=3-5) (FIG. 15H). Data are expressed as individual data points and are represented as mean±S.E.M. Significance was calculated using the Student's t test (*p<0.05, **p<0.01, and ***p<0.001).

FIGS. 16A-16E show that E2 treatment does not affect CD4⁺ T cell infiltration. FIG. 16A shows the gating strategy for identifying tumor infiltrating T cells. FIGS. 16B-16C show quantification of percentage of tumor infiltrating CD4⁺ T cells in B16F10 and YuMM5.2 tumors. FIGS. 16D-16E show quantification of percentage of tumor infiltrating CD3⁺CD4⁺ CD25^{hi}FoxP3⁺ T cells (Tregs) in B16F10 and YuMM5.2 tumors. Data are expressed as individual data points and are represented as mean±S.E.M. Significance was calculated by Student's t test (*p<0.05, **p<0.01, and ***p<0.001).

FIGS. 17A-17J show that E2 has no direct effect on T cell functionality. FIGS. 17A-17B and FIGS. 17F-17G show representative CFSE dilution plots demonstrating T cell proliferation and quantification of CFSE stain from CD8⁺ (FIGS. 17A-17B) and CD4⁺ (FIGS. 17F-17G) (n=3) cells. T cells were isolated from naive WT C57BLJ6J mice and sub-optimally activated with CD3/D28 and IL2 for 3 days in the presence or absence of E2 (1 nM) and E2 (1 nM)+fulvestrant (100 nM). FIG. 17C and FIG. 17H show the percentage of CD44⁺CD69⁺ activated CD8⁺ T cells (FIG. 17C) and CD4⁺ T cells (FIG. 17H) from experiments described above. FIG. 17D and FIG. 17I show the percentage of GZMB⁺ activated CD8⁺ T cells (FIG. 17D) and CD4⁺ T cells (FIG. 17I) from experiments described above. FIG. 17E and FIG. 17J show the percentage of IFNγ⁺ activated CD8⁺ T cells (FIG. 17E) and CD4⁺ T cells (FIG. 17J) from experiments described above. Data are expressed as individual data points and represented as mean±S.E.M. Significance was calculated by one way ANOVA.

FIGS. 18A-18M show that pharmacological depletion of ER reverses E2 dependent melanoma tumor growth. FIGS. 18A-18C show growth of B16F10 (0.5×10⁵) (n=9), YuMM5.2 (5×10⁵) (n=6), and BPD6 (5×10⁵) (n=5) tumors in ovariectomized C57BL/6J mice supplemented with placebo or E2 and co-treated with the ERα antagonist fulvestrant. FIG. 18D shows quantification of the ratio of M1 and M2 macrophages isolated from BPD6 tumors. FIGS. 18E-18G show quantification of M1 macrophages (MHCII^{hi} CD206^{-ive}), GZMB⁺CD8⁺ T cells, and PD1⁺CD8⁺ T cells in YuMM5.2 tumors from FIG. 18C (n=4). FIG. 18H shows individual volumes of BPD6 tumors implanted in ovariectomized mice treated with placebo or E2 following co-treatment with fulvestrant and ICB (anti PD1+anti CtLA4) either alone or in combination. Vehicle+IgG (n=10, red), fulvestrant+IgG (n=15, blue), vehicle+ICB (n=15, black), and fulvestrant+ICB (n=15, brown). Black arrow indicates start of ICB treatment regimen. FIG. 18I shows tumor volumes of BPD6 measured at day 12 after inoculation. FIG. 18J shows individual tumor volumes of B16F10 (0.5×10⁵) implanted in ovariectomized C57BL6/J mice supplemented with placebo and E2 and co-treated with fulvestrant along with ICB (anti-PD1). Vehicle+IgG (n=9, red), fulvestrant+IgG (n=8, blue), vehicle+ICB (n=9, black), and fulvestrant+ICB (n=10, brown). Black arrow indicates start of anti-PD1 treatment regimen. FIGS. 18K-18L show tumor volumes of B16F10 measured at day 16 (all 4 groups) and day 22 (E2+fulvestrant vs. E2+fulvestrant+anti-PD1) group after inoculation. FIG. 18M shows the median overall survival in all patients treated with immunotherapy (Pembrolizumab or Nivolumab alone, or in combination with Ipilimumab) from a previous dataset (Gide et al., Cancer Cell. 2019, 35, 238-255, incorporated herein by reference) with either high or low E2-down-regulated gene signatures derived from CD68⁺ cells in the scRNA seq. FIGS. 18A-18C are representative of two individual experiments. Data are expressed as mean 3 S.E.M. Significance was calculated by one-way ANOVA followed by Bonferroni's multiple correction (FIG. 18K), by Student's t test (FIG. 18L), and by log rank test (FIG. 18M) (*p<0.05, **p<0.01, and ***p<0.001).

FIGS. 19A-19F show that fulvestrant treatment reverses E2 induced melanoma tumor growth. FIG. 19A shows uterine wet weights of E2 treated mice bearing B16F10 tumors and treated with either vehicle or fulvestrant. FIGS. 19B-19C show weights and photographs of tumors resulting from implanting B16F10 cells in E2 supplemented C57BL/6J mice that were treated with either vehicle or fulvestrant (n=6). FIGS. 19D-19E show representative flow cytometry analysis of tumor infiltrating M2 macrophages isolated from B16F10 tumors treated with either vehicle or fulvestrant (FIG. 19D). Quantification of total CD206⁺ macrophages from the same experiment (n=6) are shown in FIG. 19E. FIG. 19F shows CFSE⁺ CD8 T cells from T cells:BMDM co-cultures where BMDM were differentiated on either normal media or tumor conditioned media and then treated with DMSO, E2 (1 nM), or E2 (1 nM)+fulvestrant (100 nM) and polarized to the M2 lineage. Data are expressed as individual data points and represented as mean±S.E.M. Significance was calculated by Student's t test (FIGS. 19A-19B and FIG. 19E) (*p<0.05).

### DETAILED DESCRIPTION

Described herein are compositions and methods for treating cancer, such as melanoma, lung cancer, breast cancer, and colon cancer. The compositions and methods may include at least one estrogen receptor (ER) modulating drug. The compositions and methods may further include at least one additional cancer therapy.

It was studied whether there are sex hormone-dependent baseline differences in the immune system that contribute to gender specific differences in tumor immunity and immune checkpoint blockade (ICB) efficacy. As detailed herein, it was explored how E2 modulates immune cell function and repertoire within the melanoma tumor microenvironment (TME) and how this influences tumor growth in established murine models of this disease. Specifically, it was discovered that a primary action of E2 is to facilitate the polarization of macrophages towards an immune-suppressive state in the tumor microenvironment, characterized by an enhanced ability to promote tumor growth and, in an indirect manner, suppress cytotoxic T cell responses. The immune-suppressive state promotes CD8+ T cell dysfunction/exhaustion and ICB resistance. This activity was not evident in mice harboring a macrophage specific depletion of ERα confirming a direct role for estrogen signaling within myeloid cells in establishing an immunosuppressed state. Further, pharmacological inhibition of E2 signaling, using the Selective Estrogen Receptor Downregulator (SERD)/antagonist fulvestrant, reversed E2 enhanced melanoma tumor growth by stimulating the establishment and maintenance of a pro-immunogenic TME characterized by increased presence of activated CD8+ T cells. In preclinical models of melanoma, fulvestrant treatment increased the efficacy of ICBs such as α-PD1 and α-CTLA4. Further, a gene signature that reads on ER activity in macrophages predicted survival in ICB treated melanoma patients. These results highlight the importance of E2/ER as a regulator of intratumoral macrophage polarization—an activity that may be therapeutically targeted to reverse immune suppression and increase ICB efficacy. Accordingly, contemporary SERDs may be combined with standard of care immunotherapies to maximize therapeutic response in melanoma patients.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

The term “administration” or “administering,” as used herein refers to providing, contacting, and/or delivery an agent or composition as detailed herein, by any appropriate route to achieve the desired effect. These agents may be administered to a subject in numerous ways and may be used in combination.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Antiprogestogens” and “antiprogestins” as used herein, are used interchangeably and refer those class of drugs/compounds that act as progesterone antagonists or progesterone blockers and prevent progestogens (for example, progesterone) from mediating their biological effects in the body of a subject.

“Aromatase inhibitor” as used herein refers to the class of compounds/drugs that target aromatase, which is an enzyme involved in the biosynthesis of estrogen. Aromatase inhibitors may block the production of estrogen or block the action of estrogen on receptors.

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Cancer” refers to all types of cancer or neoplasm or malignant tumors found in animals, including carcinoma, adenoma, melanoma, sarcoma, lymphoma, leukemia, blastoma, glioma, astrocytoma, mesothelioma, or a germ cell tumor. Cancer may include cancer of, for example, the colon, rectum, stomach, pancreas, bladder, cervix, uterus, vulva, endometrium, salivary gland, skin, epithelium, muscle, kidney, liver, lymph, thyroid, bone, blood, ovary, prostate, lung, brain, head and neck, and/or breast. Cancer may include medullablastoma, non-small cell lung cancer, small cell lung cancer, gastrointestinal, neuroblastoma, glioblastoma, peripheral neuroepithelioma, hepatoma, colorectal cancer, uterine cervical cancer, melanoma, myeloma, and/or mesothelioma. The cancer may include leukemia. The cancer may include any metastasis of the cancer. The term “leukemia” refers to broadly progressive, malignant diseases of the hematopoietic organs/systems and is generally characterized by a distorted proliferation and development of leukocytes and their precursors in the blood and bone marrow. Leukemia diseases include, for example, chronic myeloid leukemia (CML), acute myeloid leukemia (AML), acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid leukemia, myeloid granulocytic leukemia, myelomonocytic leukemia, Naegeli leukemia, plasma cell leukemia, plasmacytic leukemia, and promyelocytic leukemia. In some embodiments, the cancer is selected from melanoma, lung cancer, breast cancer, and colon cancer, and metastatic variations thereof. In some embodiments, the cancer comprises melanoma. In some embodiments, the cancer comprises breast cancer.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another. e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Effective amount” or “therapeutically effective amount” refers to an amount sufficient to effect beneficial or desirable biological and/or clinical results.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Nucleic acid” or“oligonucleotide” or“polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, mRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

A “peptide” or“polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein.” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Selective Estrogen Receptor Degrader or Downregulator” or “SERDs” are used interchangeably and refer to those class of drugs/compounds that bind to the estrogen receptor (ER) and, in the process of doing so, causes the estrogen receptor to be degraded and thus downregulated.

“Selective Estrogen Receptor Modulators” or “SERMs” refers to the class of drugs/compounds that act on the estrogen receptor (ER).

“Subject” and “patient” as used herein interchangeably refers to any vertebrate, including, but not limited to, a mammal that wants or is in need of the herein described compositions or methods. The methods and compositions disclosed herein can be used on a sample either in vitro (for example, on isolated cells or tissues) or in vivo in a subject (for example, a living organism, such as a patient). The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment. In some embodiments, the subject has cancer.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. ESTROGEN RECEPTOR (ER) MODULATING DRUG

Provided herein are estrogen receptor (ER) modulating drugs, which may also be referred to as an “ER modulator.” The term “estrogen receptor (ER) modulating drug” refers to any drug/compound, or class of drug/compound that is capable of modulating the estrogen receptor on a cell. An ER modulating drug may bind an estrogen receptor. An ER modulating drug may prevent or reduce the binding of a molecule to the estrogen receptor.

An ER modulating drug may increase and/or prolong the binding of a molecule to the estrogen receptor. An ER modulating drug may decrease or reduce the activity of the estrogen receptor. An ER modulating drug may increase or enhance the activity of the estrogen receptor.

An ER modulating drug may modulate an ER receptor by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may modulate an ER receptor by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may modulate an ER receptor by about 5-95%, 10-90%, 15-85%, 20-80%, or 1.5-fold to 10-fold, relative to a control.

An ER modulating drug may have agonist activity against an ER receptor. An ER modulating drug may increase or enhance the activity of an ER receptor by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may increase or enhance the activity of an ER receptor by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may increase or enhance the activity of an ER receptor by about 5-95%, 10-90%, 15-85%, 20-80%, or 1.5-fold to 10-fold, relative to a control.

An ER modulating drug may have antagonist activity against an ER receptor. An ER modulating drug may decrease or inhibit the activity of an ER receptor by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may decrease or inhibit the activity of an ER receptor by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may decrease or inhibit the activity of an ER receptor by about 5-95%, 10-90%, 15-85%, 20-80%, or 1.5-fold to 10-fold, relative to a control.

ER modulating drugs may comprise a small molecule, peptide, polypeptide, antibody, nucleotide, polynucleotide, lipid, or carbohydrate, or a combination thereof. ER modulating drugs may include, for example, a selective estrogen receptor modulator (SERM), a selective estrogen receptor degrader (SERD), an antiprogestin, an aromatase inhibitor, or a combination thereof. The ER may be ER-alpha, or ER-beta, or a combination thereof. An effective amount of the ER modulating drug may be administered.

a. SERM

SERMs may comprise a small molecule, peptide, polypeptide, antibody, nucleotide, polynucleotide, lipid, or carbohydrate, or a combination thereof. SERMs may be synthesized and/or extracted and/or purified by any suitable means known in the art. SERMs may be commercially available. SERMs may include, for example, lasofoxifene (FABLYNO), bazodoxifene, tamoxifen (NOLVADEX®; TAMIFENO), raloxifene (EVISTA®), toremifene (FARESTON®), orarzoxifene (also known as LY-353381), ospemifene (OSPHENA®; SENSHIO®), clomiphene (also known as clomiphene; CLOMIDO; SEROPHENE®), or H3B6545, or a combination thereof. Examples of other SERMS are described in International Patent Application No. PCT/US2015/023216 published as WD 2015/149045, U.S. Pat. Nos. 7,612,114, 7,960,412, 8,399,520, U.S. Patent Publication No. US 2009-0325930, and U.S. Patent Publication No. US 2006-0116364, the contents of which are incorporated by reference in their entirety.

b. SERD

SERDs may comprise a small molecule, peptide, polypeptide, antibody, nucleotide, polynucleotide, lipid, or carbohydrate, or a combination thereof. SERDs may be synthesized and/or extracted and/or purified by any suitable means known in the art. SERDs may be commercially available. SERDs may include, for example. ICI 182780 (also known as fulvestrant; FASLODEX®). LSZ102, LY3484356, giredestrant (also known as GDC9545), camizestrant (also known as AZD-9833), AZD9496, GDC0927, D-052, AC0682, SAR439859 (also known as amcenestrant), RAD1901 (also known as elacestrant), G1T48 (also known as rintodestrant), Zn-c5, ARV-471, or OP-1250, or a combination thereof.

c. Antiprogestin

Antiprogestins may comprise a small molecule, peptide, polypeptide, antibody, nucleotide, polynucleotide, lipid, or carbohydrate, or a combination thereof. Antiprogestins may be synthesized and/or extracted and/or purified by any suitable means known in the art. Antiprogestins may be commercially available. Antiprogestins may include, for example, mifepristone (also known as RU-488; MIFEGYNE®), asoprisnil, onapristone, or telapristone (PROELLEX®), or a combination thereof.

d. Aromatase Inhibitor

Aromatase inhibitors may comprise a small molecule, peptide, polypeptide, antibody, nucleotide, polynucleotide, lipid, or carbohydrate, or a combination thereof. Aromatase inhibitors may be synthesized and/or extracted and/or purified by any suitable means known in the art. Aromatase inhibitors may be commercially available. Aromatase inhibitors may include, for example, letrozole (FEMARA®), anastrozole (ARIMIDEX®), Exemestane (AROMASIN®), vorozole, formestane (LENTARON®), fadrozole (AFEMA®), testolactone (TESLACO), aminoglutethimide (ELIPTEN®; CYTADREN®; ORIMETEN®), androstatrienedione, or 4-androstene-3,6,17-trione (also known as 4-AT; 6-Oxo, 6-OXO™), or a combination thereof.

### 3. ADDITIONAL THERAPIES

In some embodiments, the at least one ER modulating drug is combined with at least one additional cancer therapy. As used herein, the term “standard of care treatment” or “additional therapy” or “additional treatment” are used interchangeably and refer to any other standard cancer treatments/additional cancer treatments that do not include ER modulating drugs. Additional cancer therapies may comprise a small molecule, peptide, polypeptide, antibody, nucleotide, polynucleotide, lipid, or carbohydrate, or a combination thereof. Additional cancer therapies may be synthesized and/or extracted and/or purified by any suitable means known in the art. Additional cancer therapies may be commercially available. Additional cancer therapies may include, for example, chemotherapy, immunotherapy, radiation therapy, hormone therapy, targeted drug therapy, cryoablation, and surgery, or a combination thereof. Hormone therapy, for example, may block hormone synthesis such as blocking estrogen synthesis. An effective amount of the additional therapy may be administered.

Chemotherapies may include, for example, an antimitotic agent, an alkylating agent, an antimetabolite, an antimicrotubule agent, a topoisomerase inhibitor, a cytotoxic agent, a cell cycle inhibitor, a growth factor inhibitor, a histone deacetylase (HDAC) inhibitor, or an inhibitor of a pathway that cross-talks with and activates ER transcriptional activity, or a combination thereof.

Alkylating agents may include, for example, cisplatin (PLATINOL®), oxaliplatin (ELOXATIN®), chlorambucil (LEUKERAN®), procarbazine (MATULANE®; NATULAN®), or carmustine (BiCNU®), or a combination thereof. Antimetabolites may include, for example, methotrexate (also known as amethopterin), 5-fluorouracil, cytarabine (also known as cytosine arabinoside or ara-C; CYTOSAR®), or gemcitabine (GEMZAR®), or a combination thereof. Antimicrotubule agents may include, for example, vinblastine (VELBAN®; VELBE®), or paclitaxel (TAXOL®), or a combination thereof. Topoisomerase inhibitors may include, for example, etoposide (VEPESID®), or doxorubicin (ADRIAMYCIN®; MYOCET®), or a combination thereof. Cytotoxic agents may include, for example, bleomycin (BLENOXANE®). Growth factor inhibitors may include, for example, human epidermal growth factor receptor 2 (HER2) inhibitors. HER2 inhibitors include, for example, trastuzumab (HERCEPTIN®), deruxtecan, sacitizumab, and/or ado-trastuzumab emtansine (KADCYLA®). HDAC inhibitors may include, for example, vorinostat (ZOLINZA®), romidepsin (ISTODAX®), chidamide (also known as tucidinostat; EPIDAZA®; HIYASTA™) panobinostat (FARYDAK®), belinostat (also known as BELEODAQ® or PXD101), valproic acid (DEPAKOTE®; DEPAKENE®; STAVZOR®)), mocetinostat (also known as MGCD0103), abexinostat (also known as PCI-24781), entinostat (also known as SNDX-275 or MS-275), pracinostat (also known as SB939), resminostat (also known as 4SC-201 or RAS2410), givinostat (also known as gavinostat or ITF2357), quisinostat (also known as JNJ-26481585), kevetrin, CUDC-101, AR-42, tefinostat (also known as CHR-2845), nanatinostat (also known as CHR-3996), domatinostat (also known as 4SC-202), ivaltinostat (also known as CG-200745), rocilinostat (also known as ACY-1215), or sulforaphane, or a combination thereof. Inhibitors of a pathway that cross-talks with and activates ER transcriptional activity may include, for example, a phosphoinositide 3-kinase (PI3K) inhibitor, a heat shock protein 90 (HSP90) inhibitor, or a mammalian target of rapamycin (mTOR) inhibitor, mTOR inhibitors include, for example, everolimus (AFINITOR®; VOTUBIA®; ZORTRESS®). In some embodiments, the HDAC inhibitor comprises vorinostat (ZOLINZA®). In some embodiments, the HDAC inhibitor comprises romidepsin (ISTODAX®).

In some embodiments, the entinostat is not administered with an HER2 inhibitor. In some embodiments, the HDAC inhibitor comprises entinostat with the proviso that the subject is not treated with a HER2 inhibitor.

Immunotherapies may include, for example, a checkpoint inhibitor, or denosumab (PROLIA®; XGEVA®), or a combination thereof. “Checkpoint inhibitor” or “immune checkpoint inhibitor” may also be referred to as an immune checkpoint blockade (ICB) therapy. Checkpoint inhibitors may comprise an antibody. Checkpoint inhibitors may include, for example, an antibody to programmed cell death protein 1 (PD1) (anti-PD1), or an antibody to cytotoxic T-lymphocyte-associated protein 4 (CTLA4) (anti-CTLA4), or an antibody to programmed death-ligand 1 (PDL1) (anti-PDL1), or DMXAA (sting agonist; also known as ASA404, vadimezan, or dimethylxanthone acetic acid) or a combination thereof. “Anti-PD1” refers to an antibody that binds PD1, “anti-CTLA4” refers to an antibody that binds CTLA4, and “anti-PDL1” refers to an antibody that binds PDL1. In some embodiments, the PD-1 antibody comprises pembrolizumab (KEYTRUDA®) or nivolumab (OPDIVOo®). In some embodiments, the CTLA-4 antibody comprises ipilimumab (YERVOY®).

Targeted drug therapies may include, for example, vemurafenib (ZELBORAF®), anti-EGFR targeted therapies (such as, for example, erlotinib (TARCEVA®), and/or gefitinib (IRESSA®)), a serotonin-norepinephrine reuptake inhibitor (SNRI; such as venlafaxine (EFFEXOR XR®)), a selective serotonin reuptake inhibitor (SSRI), or gabapentin (NEURONTINO), or a combination thereof.

In some embodiments, the at least one ER modulating drug is combined with anti-PD1. In some embodiments, the at least one ER modulating drug is combined with anti-CTLA4. In some embodiments, the at least one ER modulating drug is combined with anti-PD1 and anti-CTLA4.

a. Vemurafenib

In some embodiments, the at least one ER modulating drug is combined with vemurafenib (ZELBORAF®). In some embodiments, the at least one ER modulating drug is combined with anti-PD1 and vemurafenib. In some embodiments, the at least one ER modulating drug is combined with anti-CTLA4 and vemurafenib. In some embodiments, the at least one ER modulating drug is combined with anti-PD1 and anti-CTLA4 and vemurafenib.

The at least one ER modulating drug, with or without the at least one additional cancer therapy, may include, for example, the following (in the list below, “a” stands for “anti”, as in “α-PD1” referring to an antibody that binds PD1, and “α-CTLA4” referring to an antibody that binds CTLA4):
ICI 182780 alone
Lasofoxifene alone
Bazodoxifene alone
LSZ102 alone
AZD9496 alone
SAR439859 alone
RAD1901 alone
ICI182780 and α-PD1
ICI182780 and α-CTLA4
ICI 182780 and α-PD1 and α-CTLA4
LSZ102 and α-PD1
LSZ102 and α-CTLA4
LSZ102 and α-PD1 and α-CTLA4
SAR439859 and α-PD1
SAR439859 and α-CTLA4
SAR439859 and α-PD1 and α-CTLA4
Tamoxifen and α-PD1
Tamoxifen and α-CTLA4
Tamoxifen and α-PD1 and α-CTLA4
Lasofoxifene and α-PD1
Lasofoxifene and α-CTLA4
Lasofoxifene and α-PD1 and α-CTLA4
Bazodoxifene and α-PD1
Bazodoxifene and α-CTLA4
Bazodoxifene and α-PD1 and α-CTLA4
AZD9496 and α-PD1
AZD9496 and α-CTLA4
AZD9496 and α-PD1 and α-CTLA4
AZD9496 and vemurafenib and α-PD1
AZD9496 and vemurafenib and α-CTLA4
AZD9496 and vemurafenib and α-PD1 and α-CTLA4
ICI182780 and vemurafenib and α-PD1
ICI182780 and vemurafenib and α-CTLA4
ICI 182780 and vemurafenib and α-PD1 and α-CTLA4
LSZ102 and vemurafenib and α-PD1
LSZ102 and vemurafenib and α-CTLA4
LSZ102 and vemurafenib and α-PD1 and α-CTLA4
SAR439859 and vemurafenib and α-PD1
SAR439859 and vemurafenib and α-CTLA4
SAR439859 and vemurafenib and α-PD1 and α-CTLA4
Tamoxifen and vemurafenib and α-PD1
Tamoxifen and vemurafenib and α-CTLA4
Tamoxifen and vemurafenib and α-PD1 and α-CTLA4
Lasofoxifene and vemurafenib and α-PD1
Lasofoxifene and vemurafenib and α-CTLA4
Lasofoxifene and vemurafenib and α-PD1 and α-CTLA4
Bazodoxifene and vemurafenib and α-PD1
Bazodoxifene and vemurafenib and α-CTLA4
Bazodoxifene and vemurafenib and α-PD1 and α-CTLA4
Rad1901 and vemurafenib and α-PD1
Rad1901 and vemurafenib and α-CTLA4
Rad1901 and vemurafenib and α-PD1 and α-CTLA4
G1T48 alone
G1T48 and α-PD1
G1T48 and α-CTLA4
G1T48 and α-PD1 and α-CTLA4
G1T48 and vemurafenib and α-PD1
G1T48 and vemurafenib and α-CTLA4
G1T48 and vemurafenib and α-PD1 and α-CTLA4
Raloxifene and α-PD1
Raloxifene and α-CTLA4
Raloxifene and α-PD1 and α-CTLA4
Raloxifene and vemurafenib and α-PD1
Raloxifene and vemurafenib and α-CTLA4
Raloxifene and vemurafenib and α-PD1 and α-CTLA4
Clomiphene alone
Clomiphene and α-PD1
Clomiphene and α-CTLA4
Clomiphene and α-PD1 and α-CTLA4
Clomiphene and vemurafenib and α-PD1
Clomiphene and vemurafenib and α-CTLA4
Clomiphene and vemurafenib and α-PD1 and α-CTLA4
Ospemiphene alone
Ospemiphene and α-PD1
Ospemiphene and α-CTLA4
Ospemiphene and α-PD1 and α-CTLA4
Ospemiphene and vemurafenib and α-PD1
Ospemiphene and vemurafenib and α-CTLA4
Ospemiphene and vemurafenib and α-PD1 and α-CTLA4
Arzoxifene alone
Arzoxifene and α-PD1
Arzoxifene and α-CTLA4
Arzoxifene and α-PD1 and α-CTLA4
Arzoxifene and vemurafenib and α-PD1
Arzoxifene and vemurafenib and α-CTLA4
Arzoxifene and vemurafenib and α-PD1 and α-CTLA4
Toremifene alone
Toremifene and α-PD1
Toremifene and α-CTLA4
Toremifene and α-PD1 and α-CTLA4
Toremifene and vemurafenib and α-PD1
Toremifene and vemurafenib and α-CTLA4
Toremifene and vemurafenib and α-PD1 and α-CTLA4
Zn-c5 alone
Zn-c5 and α-PD1
Zn-c5 and α-CTLA4
Zn-c5 and α-PD1 and α-CTLA4
Zn-c5 and vemurafenib and α-PD1
Zn-c5 and vemurafenib and α-CTLA4
Zn-c5 and vemurafenib and α-PD1 and α-CTLA4
ARV471 alone
ARV471 and α-PD1
ARV471 and α-CTLA4
ARV471 and α-PD1 and α-CTLA4
ARV471 and vemurafenib and α-PD1
ARV471 and vemurafenib and α-CTLA4
ARV471 and vemurafenib and α-PD1 and α-CTLA4
OP-1250 alone
OP-1250 and α-PD1
OP-1250 and α-CTLA4
OP-1250 and α-PD1 and α-CTLA4
OP-1250 and vemurafenib and α-PD1
OP-1250 and vemurafenib and α-CTLA4
OP-1250 and vemurafenib and α-PD1 and α-CTLA4
H3B6545 alone
H3B6545 and α-PD1
H3B6545 and α-CTLA4
H3B6545 and α-PD1 and α-CTLA4
H3B6545 and vemurafenib and α-PD1
H3B6545 and vemurafenib and α-CTLA4
H3B6545 and vemurafenib and α-PD1 and α-CTLA4
LY3484356 alone
LY3484356 and α-PD1
LY3484356 and α-CTLA4
LY3484356 and α-PD1 and α-CTLA4
LY3484356 and vemurafenib and α-PD1
LY3484356 and vemurafenib and α-CTLA4
LY3484356 and vemurafenib and α-PD1 and α-CTLA4
Giredestrant alone
Giredestrant and α-PD1
Giredestrant and α-CTLA4
Giredestrant and α-PD1 and α-CTLA4
Giredestrant and vemurafenib and α-PD1
Giredestrant and vemurafenib and α-CTLA4
Giredestrant and vemurafenib and α-PD1 and α-CTLA4
Camizestrant alone
Camizestrant and α-PD1
Camizestrant and α-CTLA4
Camizestrant and α-PD1 and α-CTLA4
Camizestrant and vemurafenib and α-PD1
Camizestrant and vemurafenib and α-CTLA4
Camizestrant and vemurafenib and α-PD1 and α-CTLA4
GDC0927alone
GDC0927 and α-PD1
GDC0927 and α-CTLA4
GDC0927 and α-PD1 and α-CTLA4
GDC0927 and vemurafenib and α-PD1
GDC0927 and vemurafenib and α-CTLA4
GDC0927 and vemurafenib and α-PD1 and α-CTLA4
D-052 alone
D-052 and α-PD1
D-052 and α-CTLA4
D-052 and α-PD1 and α-CTLA4
D-052 and vemurafenib and α-PD1
D-052 and vemurafenib and α-CTLA4
D-052 and vemurafenib and α-PD1 and α-CTLA4
AC0682 alone
AC0682 and α-PD1
AC0682 and α-CTLA4
AC0682 and α-PD1 and α-CTLA4
AC0682 and vemurafenib and α-PD1
AC0682 and vemurafenib and α-CTLA4
AC0682 and vemurafenib and α-PD1 and α-CTLA4
Letrozole alone
Letrozole and α-PD1
Letrozole and α-CTLA4
Letrozole and α-PD1 and α-CTLA4
Letrozole and vemurafenib and α-PD1
Letrozole and vemurafenib and α-CTLA4
Letrozole and vemurafenib and α-PD1 and α-CTLA4
Anastrozole alone
Anastrozole and α-PD1
Anastrozole and α-CTLA4
Anastrozole and α-PD1 and α-CTLA4
Anastrozole and vemurafenib and α-PD1
Anastrozole and vemurafenib and α-CTLA4
Anastrozole and vemurafenib and α-PD1 and α-CTLA4
Exemestane alone
Exemestane and α-PD1
Exemestane and α-CTLA4
Exemestane and α-PD1 and α-CTLA4
Exemestane and vemurafenib and α-PD1
Exemestane and vemurafenib and α-CTLA4
Exemestane and vemurafenib and α-PD1 and α-CTLA4
Vorozole alone
Vorozole and α-PD1
Vorozole and α-CTLA4
Vorozole and α-PD1 and α-CTLA4
Vorozole and vemurafenib and α-PD1
Vorozole and vemurafenib and α-CTLA4
Vorozole and vemurafenib and α-PD1 and α-CTLA4
Formestane alone
Formestane and α-PD1
Formestane and α-CTLA4
Formestane and α-PD1 and α-CTLA4
Formestane and vemurafenib and α-PD1
Formestane and vemurafenib and α-CTLA4
Formestane and vemurafenib and α-PD1 and α-CTLA4
Fadrozole alone
Fadrozole and α-PD1
Fadrozole and α-CTLA4
Fadrozole and α-PD1 and α-CTLA4
Fadrozole and vemurafenib and α-PD1
Fadrozole and vemurafenib and α-CTLA4
Fadrozole and vemurafenib and α-PD1 and α-CTLA4
Testolactone alone
Testolactone and α-PD1
Testolactone and α-CTLA4
Testolactone and α-PD1 and α-CTLA4
Testolactone and vemurafenib and α-PD1
Testolactone and vemurafenib and α-CTLA4
Testolactone and vemurafenib and α-PD1 and α-CTLA4
Aminoglutethimide alone
Aminoglutethimide and α-PD1
Aminoglutethimide and α-CTLA4
Aminoglutethimide and α-PD1 and α-CTLA4
Aminoglutethimide and vemurafenib and α-PD1
Aminoglutethimide and vemurafenib and α-CTLA4
Aminoglutethimide and vemurafenib and α-PD1 and α-CTLA4
Andostatrienedione alone
Androstatrienedione and α-PD1
Androstatrienedione and α-CTLA4
Androstatrienedione and α-PD1 and α-CTLA4
Androstatrienedione and vemurafenib and α-PD1
Androstatrienedione and vemurafenib and α-CTLA4
Androstatrienedione and vemurafenib and α-PD1 and α-CTLA4
6-OXO alone
6-OXO and α-PD1
6-OXO and α-CTLA4
6-OXO and α-PD1 and α-CTLA4
6-OXO and vemurafenib and α-PD1
6-OXO and vemurafenib and α-CTLA4
6-OXO and vemurafenib and α-PD1 and α-CTLA4
Mifepristone alone
Mifepristone and α-PD1
Mifepristone and α-CTLA4
Mifepristone and α-PD1 and α-CTLA4
Mifepristone and vemurafenib and α-PD1
Mifepristone and vemurafenib and α-CTLA4
Mifepristone and vemurafenib and α-PD1 and α-CTLA4
Asoprisnil alone
Asoprisnil and α-PD1
Asoprisnil and α-CTLA4
Asoprisnil and α-PD1 and α-CTLA4
Asoprisnil and vemurafenib and α-PD1
Asoprisnil and vemurafenib and α-CTLA4
Asoprisnil and vemurafenib and α-PD1 and α-CTLA4
Onapristone alone
Onapristone and α-PD1
Onapristone and α-CTLA4
Onapristone and α-PD1 and α-CTLA4
Onapristone and vemurafenib and α-CTLA4
Onapristone and vemurafenib and α-PD1
Onapristone and vemurafenib and α-PD1 and α-CTLA4
Telapristone alone
Telapristone and α-PD1
Telapristone and α-CTLA4
Telapristone and α-PD1 and α-CTLA4
Telapristone and vemurafenib and α-PD1
Telapristone and vemurafenib and α-CTLA4
Telapristone and vemurafenib and α-PD1 and α-CTLA4

### 4. PHARMACEUTICAL COMPOSITIONS

Further provided herein are pharmaceutical compositions comprising the above-described ER modulating drug(s). The pharmaceutical composition may further include at least one additional cancer therapy. In some embodiments, the pharmaceutical composition may comprise about 1 ng to about 10 mg of ER modulating drug, or about 1 ng to about 10 mg of ER modulating drug and additional cancer therapy. The ER modulating drug as detailed herein, with or without at least one additional cancer therapy, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal oil, vegetable oil, mineral oil or synthetic oil. Physiological saline solution, dextrose or other saccharide solution or glycols such as ethylene glycol, propylene glycol or polyethylene glycol can also be included. For parenteral administration, the ER modulating drug will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. Pharmaceutical compositions for vaginal topical administration can be in the form of ointment, cream, gel or lotion. The pharmaceutical compositions for vaginal topical administration often include water, alcohol, animal oil, vegetable oil, mineral oil or synthetic oil. Hydrocarbon (paraffin), wool fat, beeswax, macrogols, emulsifying wax or cetrimide can also be included.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition at a concentration less than 6 mg/mL.

The ER modulating drug and/or additional cancer therapy may be present or formulated as a pharmaceutically acceptable salt thereof, or a prodrug thereof. The term “pharmaceutically acceptable salt” refers to non-toxic pharmaceutically acceptable salts (see Gould, International Journal of Pharmaceutics 1986, 33, 201-217; and Berge et al., Journal of Pharmaceutical Sciences 1977, 66, 1-19). Other salts well known to those in the art may, however, be used. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

Embodiments also include prodrugs of the compounds disclosed herein. In general, such prodrugs will be functional derivatives of the compounds which are readily convertible in vivo into the required compound. Thus, in the methods of treatment of the present invention, the term ‘administering’ shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, H. Bundgaard, Elsevier, 1985.

Some of the crystalline forms for the compounds may exist as polymorphs and as such are intended to be included in the present invention. In addition, some of the compounds may form solvates with water (i.e., hydrates) or common organic solvents, and such solvates are intended to be encompassed by some embodiments.

Where the processes for the preparation of the compounds as disclosed herein give rise to mixtures of stereoisomers, these isomers may be separated by conventional techniques such as preparative chromatography. The compounds may be prepared in racemic form or as individual enantiomers or diastereomers by either stereospecific synthesis or by resolution. The compounds may, for example, be resolved into their component enantiomers or diastereomers by standard techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral HPLC column. It is to be understood that all stereoisomers, racemic mixtures, diastereomers, cis-trans isomers, and enantiomers thereof are encompassed by some embodiments.

In embodiments wherein the pharmaceutical composition comprises both the at least one ER modulating drug and the at least one additional cancer therapy, they may be present in the pharmaceutical composition in a variety of molar ratios. The molar ratio between the at least one ER modulating drug and the at least one additional cancer therapy may be 1:1, or 1:15, or from 5:1 to 1:10, or from 1:1 to 1:5. The molar ratio between the at least one ER modulating drug and the at least one additional cancer therapy may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the at least one ER modulating drug and the at least one additional cancer therapy may be less than 20:1, less than 15:1, less than 10:1, less than 9:1, less than 8:1, less than 7:1, less than 6:1, less than 5:1, less than 4:1, less than 3:1, less than 2:1, or less than 1:1.

### 5. ADMINISTRATION

The ER modulating drug as detailed herein, with or without at least one additional cancer therapy as detailed herein, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed ER modulating drug, with or without at least one additional cancer therapy, or compositions comprising the same, may be administered to a subject by different routes including orally, ocularly, nasally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In some embodiments, administration is via aerosol or suppository. In certain embodiments, the ER modulating drug, with or without at least one additional cancer therapy, or compositions comprising the same, is administered to a subject orally, intravenously, vaginally, or transdermally, or a combination thereof. The composition may be injected into any organ or tissue of the subject. In some embodiments, the ER modulating drug, with or without at least one additional cancer therapy, or compositions comprising the same, is administered to the subject by vaginal ring administration.

In some embodiments, the ER modulating drug is administered either alone or in combination with one or more additional therapies. The at least one ER modulating drug and the at least one additional cancer therapy may be administered in a variety of molar ratios. The molar ratio between the at least one ER modulating drug and the at least one additional cancer therapy may be 1:1, or 1:15, or from 5:1 to 1:10, or from 1:1 to 1:5. The molar ratio between the at least one ER modulating drug and the at least one additional cancer therapy may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the at least one ER modulating drug and the at least one additional cancer therapy may be less than 20:1, less than 15:1, less than 10:1, less than 9:1, less than 8:1, less than 7:1, less than 6:1, less than 5:1, less than 4:1, less than 3:1, less than 2:1, or less than 1:1.

In some embodiments, the ER modulating drug is administered to the subject by oral administration (orally, or “os”). In certain embodiments, ER modulating drug is administered at about 0.5 mg/day per os to about 10 mg/day per os, such as about 0.5 mg/day per os to about 5 mg/day per os, about 0.5 mg/day per os to about 5 mg/day per os, about 1 mg/day per os to about 5 mg/day per os, about 2 mg/day per os to about 5 mg/day per os, about 3 mg/day per os to about 5 mg/day per os, about 4 mg/day per os to about 5 mg/day per os, about 0.5 mg/day per os to about 4 mg/day per os, about 1 mg/day per os to about 4 mg/day per os, about 2 mg/day per os to about 4 mg/day per os, about 3 mg/day per os to about 4 mg/day per os, about 0.5 mg/day per os to about 3 mg/day per os, about 1 mg/day per os to about 3 mg/day per os, about 2 mg/day per os to about 3 mg/day per os, about 0.5 mg/day per os to about 2 mg/day per os, about 1 mg/day per os to about 2 mg/day per os, or about 0.5 mg/day per os to about 1 mg/day per os. In some embodiments, the ER modulating drug is administered at about 0.5 mg/day per os. In some embodiments, the ER modulating drug is administered at about 1 mg/day per os. In some embodiments, the ER modulating drug is administered at about 1.5 mg/day per os. In some embodiments, the ER modulating drug is administered at about 2 mg/day per os. In some embodiments, the ER modulating drug is administered at about 2.5 mg/day per os. In some embodiments, the ER modulating drug is administered at about 3 mg/day per os. In some embodiments, the ER modulating drug is administered at about 3.5 mg/day per os. In some embodiments, the ER modulating drug is administered at about 4 mg/day per os. In some embodiments, the ER modulating drug is administered at about 4.5 mg/day per os. In some embodiments, the ER modulating drug is administered at about 5 mg/day per os. In some embodiments, the ER modulating drug is administered at about 6 mg/day per os. In some embodiments, the ER modulating drug is administered at about 7 mg/day per os. In some embodiments, the ER modulating drug is administered at about 8 mg/day per os. In some embodiments, the ER modulating drug is administered at about 9 mg/day per os. In some embodiments, the ER modulating drug is administered at about 10 mg/day per os. In some other embodiments, the ER modulating drug is administered at more than 10 mg/day per os.

In certain embodiments, when the ER modulating drug is administered to a subject whose cancer has not acquired endocrine resistance, the ER modulating drug can be administered at less than 0.5 mg/day per os for prevention of endocrine resistance. In certain embodiments, when the ER modulating drug is administered to cancer patient as adjuvant treatment, the ER modulating drug can be administered at less than 0.5 mg/day per os for prevention of endocrine resistance.

A composition can be administered alone or in combination with other treatments, either simultaneously or sequentially, dependent upon the condition to be treated. The at least one ER modulating drug and the at least one additional therapy may be administered together or simultaneously, they may be administered at different times or sequentially. The at least one ER modulating drug and the at least one additional therapy and the Vemurafenib may be administered simultaneously or sequentially.

The at least one ER modulating drug, with or without the at least one additional therapy, may be administered to the subject once every day, once every 2 days, once every 3 days, once every 4 days, once every 5 days, once every 6 days, once every 7 days, once every week, once every 2 weeks, once every 3 weeks, once every 4 weeks, once every 5 weeks, once every 6 weeks, once every 7 weeks, once every 8 weeks, once every month, once every 2 months, once every 3 months, once every 4 months, once every 5 months, or once every 6 months. The at least one ER modulating drug may be administered to the subject for 1 year, 2 years, 3 years, 4 years, 5 years, or more than 5 years. In some embodiments, the ER modulating drug, with or without the at least one additional therapy, is administered to the subject until the subject's cancer progresses on therapy.

### 6. METHODS

a. Methods of Treating Cancer

Provided herein are methods of treating cancer. Provided herein are methods of treating cancer in a subject in need thereof. The methods may include administering to the subject at least one ER modulating drug, as detailed herein. The methods may further include administering to the subject at least one additional therapy, as detailed herein.

In some embodiments, the at least one ER modulating drug is administered with anti-PD1. In some embodiments, the at least one ER modulating drug is administered with anti-CTLA4. In some embodiments, the at least one ER modulating drug is administered with anti-PD1 and anti-CTLA4. In some embodiments, the at least one ER modulating drug is administered with anti-PD1 and Vemurafenib. In some embodiments, the at least one ER modulating drug is administered with anti-CTLA4 and Vemurafenib. In some embodiments, the at least one ER modulating drug is administered with anti-PD1, anti-CTLA4, and Vemurafenib.

The compositions and methods detailed herein may have a variety of effects in the subject, relative to a control. Tumor-associated macrophage (TAM) polarization towards an immune suppressive phenotype may be reduced. ER-alpha in myeloid cells may be depleted. The Wnt 5A/TCF4 pathway may be reduced. CD4+ T cell infiltration may not be affected. Interferon pathways may be reduced. CD8⁺ T cell proliferation may be increased. CD8+ T cell migration may be increased. CD8+ T cell cytotoxicity may be increased. The ratio of M1/M2 macrophages may be increased. Tumor growth may be decreased. Tumor size may be decreased. Circulating tumor cells may be reduced. Cancer metastasis may be reduced.

The at least one ER modulating drug, or the at least one additional therapy, or a combination thereof, may treat cancer. The at least one ER modulating drug, or the at least one additional therapy, or a combination thereof, may reduce cancer. Reducing cancer may include reducing tumor size, reducing tumor growth, reducing cancer metastasis, or a combination thereof. In some embodiments, the at least one ER modulating drug, or the at least one additional therapy, or a combination thereof, reduces cancer by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The cancer may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The cancer may be reduced by about 5-95%, 10-90%, 15-85%, 20-80%, or 1.5-fold to 10-fold, relative to a control.

b. Methods of Improving Effectiveness of ICB Therapies

Provided herein are methods of improving the effectiveness of ICB therapies. The methods may include administering to the subject at least one ER modulating drug, as detailed herein. The methods may further include administering to the subject at least one additional therapy, as detailed herein.

Provided herein are methods of treating cancer in a subject. The method may include administering to the subject at least one estrogen receptor (ER) modulating drug such that the effectiveness of an ICB therapy is increased relative to a control. The method may further include administering to the subject the ICB therapy. In some embodiments, the ICB therapy is selected from anti-PD1, anti-CTLA4, anti-PDL1, and DMXAA, or a combination thereof. In some embodiments, the method further comprises administering to the subject at least one additional therapy.

In some embodiments, the at least one ER modulating drug is administered with anti-PD1. In some embodiments, the at least one ER modulating drug is administered with anti-CTLA4. In some embodiments, the at least one ER modulating drug is administered with anti-PD1 and anti-CTLA4. In some embodiments, the at least one ER modulating drug is administered with anti-PD1 and vemurafenib. In some embodiments, the at least one ER modulating drug is administered with anti-CTLA4 and vemurafenib. In some embodiments, the at least one ER modulating drug is administered with anti-PD1, anti-CTLA4, and vemurafenib.

The at least one ER modulating drug, or the at least one additional therapy, or a combination thereof, may increase the effectiveness of an ICB therapy. In some embodiments, the at least one ER modulating drug, or the at least one additional therapy, or a combination thereof, increases the effectiveness of an ICB therapy by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The effectiveness of an ICB therapy may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The effectiveness of an ICB therapy may be increased by about 5-95%, 10-90%, 15-85%, 20-80%, or 1.5-fold to 10-fold, relative to a control.

c. Methods of Predicting Response of a Subject to ICB Therapy

Provided herein are methods of predicting response of a subject to ICB therapy. The method may include determining the level of expression in the subject of a gene selected from “Genes up-regulated upon E2 treatment” in TABLE 5 and “Genes down-regulated upon E2 treatment” in TABLE 5. In some embodiments, the level of expression of the gene selected from “Genes up-regulated upon E2 treatment” is decreased relative to a control. In some embodiments, the level of expression of the gene selected from “Genes down-regulated upon E2 treatment” is increased relative to a control. The method may further include identifying the subject as responsive to ICB therapy. In some embodiments, the method further includes administering to the subject at least one ICB therapy. In some embodiments, the at least one ICB therapy is selected from anti-PD1, anti-CTLA4, anti-PDL1, and DMXAA, or a combination thereof.

In some embodiments, the expression of the gene is increased by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5-95%, 10-90%, 15-85%, 20-80%, or 1.5-fold to 10-fold, relative to a control. In some embodiments, the expression of the gene is decreased by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be decreased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be decreased by about 5-95%, 10-90%, 15-85%, 20-80%, or 1.5-fold to 10-fold, relative to a control. The level of expression of the gene in the subject, or in a sample therefrom, may be determined by any suitable means known in the art, which may include, for example, antibody binding, Western blot analyses, Northern blot hybridization analyses, hybridization of a probe to a gene transcript such as on a microarray, amplification-based detection methods such as reverse-transcription based polymerase chain reaction (RT-PCR) or quantitative RT-PCR or RNA sequencing, or a combination thereof. The expression level of the genes can be analyzed based on the biological activity or quantity of proteins encoded by the genes. The gene expression levels may be determined by measuring mRNA or protein levels of the genes. The protein levels of a biomarker may be determined using proteomics, immunoassay, enzyme-linked immunoassay (ELISA), radioimmunoassay (RIA), a competitive inhibition assay such as forward or reverse competitive inhibition assays, a fluorescence polarization assay, a competitive binding assay, or a combination thereof.

### 7. Examples

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Mice. C57BL/BJ, LysMCre (B6.129P2-Lyz2^{tm1(cre)lfo}/J) Pmel (B6.Cg-Thy1^{a}/Cy Tg(TcraTcrb)8Rest/J) mice were from Jackson Laboratories (Bar Harbor, ME). Age matched mice were used for all the studies. LysMCre mice were bred to Esr1^{f/f} mice to generate Esr1^{f/f}; LysMCre and littermate control LysMCre and Esr1^{f/f} mice. iBP (Braf^{V600E/WT}, Pten^{f/f}mTyrCreERT2) mice were generated by crossing breeders Braf^{WT/WT}/Pten^{f/f}, mTyrCreERT2 mice to BRAF^{V600E}, Pten^{f/f} mice. The mice were housed in secure animal facility cages in 12 hour light:dark cycles at temperature around 25° C. and 70% humidity. Mice had access to ad-libitum food and water. NSG (NOD.Cg-Prkd^{scid} Il2rg^{tm1Wjl}/SzJ) mice were from the Division of Laboratory Animal Resources (Duke University). The NSG animals were fed with a GL3 diet and were kept in pathogen free conditions.

Tumor models and cells. The mouse B16F10 and Yumm5.2 cell lines were purchased from American Type Culture Collection (ATCC, Manassas, VA). The mouse melanoma cell line BPD6 was established from iBP as described elsewhere (Zhao et al., Immunity 2018, 48, 147-160). B16F10 and BPD6 cells were maintained in Dulbecco's Modified Eagle's Medium (DMEM) supplemented with 8% fetal bovine serum (FBS), 0.1 mM non-essential amino acids (NEAA) and 1 mM sodium pyruvate. YuMM5.2 cells were maintained in DMEM/F12 media supplemented with 10% FBS. The cells were replated 3 times/week at a confluency of 1:10 and were kept in a 37° C. incubator at 5% CO2. For subcutaneous tumor models B16F10 (5×10⁴ or 1×10⁵), YuMM5.2 (5×10⁵), and BPD6 (5×10⁵) cells were injected into the right flank of the mice. For iBP mice, tumors were induced with a single intradermal dose of 4-hydroxytamoxifen in DMSO (150 μg/mouse). Tumors were measured thrice weekly using an electronic caliper. Tumor volumes were calculated by the formula V=L×(W×W)/2. For iBP mouse tumors, volumes were calculated by the formula V=L×B×H. For tumor growth rate studies mice were euthanized when the tumors reached a maximum size of 2000 mm³.

Ovarlectomy and subcutaneous pellet Insertion. Ovariectomy was performed as previously described (Nelson et al., Science 2013, 342, 1094-1098). Eight days prior to tumor inoculation, 7 weeks old C57BL/6J or 5 weeks old (iBP) female mice were subjected to ovariectomy or sham surgery. Mice were anesthetized in an inhalation chamber (2% Isoflurane) and maintained in half the dose of isoflurane (1%) via nose cone throughout the surgical process. Prior to surgery, mice were administered a 5 mg/kg dose of carprofen subcutaneously. The area below the ribs was shaved with an electronic razor and the skin was sterilized by rubbing with betadine and alcohol (3× alternating). A horizontal incision was made through the skin above the ovary fat pad, followed by a vertical incision through the abdominal muscle wall. The ovary was externalized and removed using a cauterizing scissors (or returned if sham). Fat pad was replaced, and muscle walls were opposed and sutured (1-2 stitches). Following suturing, 1 drop of bupivacaine (0.25%) was added on top of the incision site. The skin was opposed, and a wound clip was placed on the incision site. This was repeated for the other ovary. The mouse was then removed from anesthesia and kept in a clean cage and monitored until conscious. The mice were monitored for recovery for 10 days. On day 7 after surgery, the mice were supplemented with either placebo or E2 (0.01 mg/60 days continuous release, Innovative Research of America, FL, Sarasota) pellets with the help of a trocar.

Single Cell RNA sequencing. iBP tumors (three) were pooled and a single cell suspension was isolated. Live tumor infiltrating immune cells (CD45⁺ L/D⁻) were isolated by cell sorting and resuspended in PBS+0.04% BSA at a concentration of 1000 cells/μL. In total, 10,000 cells were loaded on the 10× Genomics Chromium Controller Single-Cell Instrument (10× Genomics) mixed with reverse transcription reagents along with gel beads and oil to generate single-cell gel beads in emulsions (GEMs). GEM-RT was performed in an Eppendorf Mastercycler Pro (cat #950030020, Eppendorf): 53° C. for 45 min; 85° C. for 5 min; then held at 4° C. After reverse transcription, GEMs were disrupted and the single-stranded cDNA was purified using Dynabeads MyOne Silane beads (cat #37002D, Thermo Fisher Scientific). cDNA was amplified using the Eppendorf Mastercycler Pro (cat #950030020, Eppendorf): 98° C. for 3 min; cycled 11×: 98° C. for 15 s, 67° C. for 20 s, and 72° C. for 1 min; 72° C. for 1 min; held at 4° C. The amplified cDNA product was purified with the SPRIselect Reagent Kit (0.6×SPRI) (cat #B23318, Beckman Coulter). Indexed sequencing libraries were constructed using the reagents in the Chromium Single-Cell 3′ version 3 Library Kit, following these steps: (1) fragmentation, end repair and A-tailing; (2) SPRIselect cleanup; (3) adapter ligation; (4) post ligation cleanup with SPRIselect; (5) sample index PCR; (6) PostindexPCR cleanup. The barcoded sequencing libraries were analyzed using quantitative PCR (cat #KK4824, KAPA Biosystems Library Quantification Kit for Illumina platforms). Sequencing libraries were transferred to the Duke University Center for Genomic and Computational Biology (GCB) and were loaded on a NovaSeq6000 (Illumina) for sequencing.

scRNA seq data analysis. Sequencing data was de-multiplexed, trimmed, filtered, aligned, and quantified using the Cell Ranger pipeline (10× Genomics). Reads were aligned to CSC mm10 transcriptome and UMI count matrices for each sample was obtained. The Seurat v3.1 package was used to count matrices. For each sample, cells that express <200 or >2000 genes and cells that express >5% of mitochondrial genes were removed. Highly variable genes were identified and used for principal component analysis. Cell subpopulations were identified using the ‘FindNeighbors’ function with first 30 PCs and ‘FindClusters’ function from Seurat R package with default resolution parameters. Cells were then clustered and visualized using uniform manifold approximation and projection (UMAP) (Liu, Int. J. Mol. Sci. 2020, 21, 16). DE Wilcoxon test analysis was used to identify genes that define a cluster using known cell type signatures and genes that differ within clusters between treatments.

Pseudotime Analysis. To infer the developmental trajectories in the monocyte/macrophage lineages, Monocle3 was used to perform the pseudotime analysis where UMAP coordinates from Seurat were used as input (Cao et al., Nature 2019, 566, 498-502). The graphtest function implemented in Monocle3 was used to find genes that vary with pseudotime where genes with q<0.01 were identified as pseudotime-dependent genes. Cells were divided into four pseudotime blocks (e.g., 0-5, 5-10, 10-15, and 15-21) based on their pseudotime estimate.

Immunoblotting. Cells were washed three times with 2 mL of ice-cold PBS and lysed with 0.15 mL of phospho-RIPA lysis buffer (Tris-HCl pH 7.5, 50 mM; NaCl, 150 mM; NP-40, 1%; Sodium deoxycholate, 0.5%; SDS, 0.05%; EDTA, 5 mM; Sodium fluoride, 50 mM; Sodium pyrophosphate, 15 mM; R-glycerophosphate, 10 mM; Sodium orthovanadate, 1 mM) with protease inhibitor cocktail (Millipore-Sigma, P-8340) 50 μg of B16F10 and YuMM5.2 cell lysates and 25 μg of MCF7 cell lysates were denatured and resolved by SDS-PAGE. Proteins were transferred to Odyssey Nitrocellulose Membrane (Cat no #926-31092. LI-COR Biosciences). Primary antibodies used were anti-ERα (6F11, 1:1000, Leica cat #6F11), anti-actin (Cell Signaling, cat no #8457, dilution 1:20000). Secondary antibodies used were HRP-conjugated anti-mouse IgG (1:5000) Catalog #1706516 and anti-rabbit IgG (1:5000) Catalog #1706515, BioRad) and protein bands were visualized by Western Lightning Plus Ecl system (Catalog #ORT2655 and ORT2755 Perkin Elmer).

Quantitative PCR of tumor Infiltrating myeloid cells. Tumor infiltrating myeloid cells were isolated from iBP tumors using a CD11b isolation kit (catalog #18970, StemCell Technologies). RNA was isolated using RNA Aqueous Micro kit (catalog #1931, Ambion) followed by cDNA synthesis using an iScript cDNA synthesis kit (Cat #170-7691). Quantitative amplification was performed using Sybr Green (Cat #1725124, Bio-Rad) and a CFX-384 Real Time PCR detection system. Primers used are listed below in TABLE 1.

TABLE 1
Details of qPCR primers (mouse).

| Gene | | |
|---|---|---|
| ID | Forward Primer | Reverse Primer |
| Hif1 | CCTGCACTGAATCAAGAGGTTGC | CCATCAGAAGGACTTGCTGGCT |
| | (SEQ ID NO: 1) | (SEQ ID NO: 2) |
| Jak2 | GCTACCAGATGGAAACTGTGCG | GCCTCTGTAATGTTGGTGAGATC |
| | (SEQ ID NO: 3) | (SEQ ID NO: 4) |
| B4galt | GCAACTCGACTATGGCATCTACG | CGGAATGAGGTCCACATCACTG |
| | (SEQ ID NO: 5) | (SEQ ID NO: 6) |
| Stat1 | GCCTCTCATTGTCACCGAAGAAC | TGGCTGACGTTGGAGATCACCA |
| | (SEQ ID NO: 7) | (SEQ ID NO: 8) |
| Zeb2 | GCAGTGAGCATCGAAGAGTACC | GGCAAAAGCATCTGGAGTTCCAG |
| | (SEQ ID NO: 9) | (SEQ ID NO: 10) |
| Tgm2 | GAAGGAACACGGCTGTCAGCAA | GATGAGCAGGTTGCTGTTCTGG |
| | (SEQ ID NO: 11) | (SEQ ID NO: 12) |
| Tspo | GAGCCTACTTTGTACGTGGCGA | GCTCTTTCCAGACTATGTAGGAG |
| | (SEQ ID NO: 13) | (SEQ ID NO: 14) |
| Itm2b | CATCAGTGTGCCTGTACCAGAG | GAGGAATCACGTAGCACTTGTCC |
| | (SEQ ID NO: 15) | (SEQ ID NO: 16) |
| Vegfa | CTGCTGTAACGATGAAGCCCTG | GCTGTAGGAAGCTCATCTCTCC |
| | (SEQ ID NO: 17) | (SEQ ID NO: 18) |
| Lcp1 | TCTGTGCCAGACACGATTGACG | GAGGCAGAGTTCAGAGCCAAGT |
| | (SEQ ID NO: 19) | (SEQ ID NO: 20) |
| Tof7 | CCTCTCATCACCTACAGCAACG | CTGGAGACAGTGGGTAATACGG |
| | (SEQ ID NO: 21) | (SEQ ID NO: 22) |
| Esr1 | AGGTGCCCTACTACCTGGAG (SEQ | GTCTCTCTCGGCCATTCTGG (SEQ |
| | ID NO: 23) | ID NO: 24) |
| Cxcl12 | CATCCATCCATCCATCCA (SEQ ID | TTCAGGGTCATGGAGACAGT (SEQ |
| | NO: 25) | ID NO: 26) |
| Pgr | AGGTCTACCCGCCATACCTT (SEQ | CGCCATAGTGACAGCCAGAT (SEQ |
| | ID NO: 27) | ID NO: 28) |
| Rplpo | AGATTCGGGATATGCTGTTGGC | TCGGGTCCTAGACCAGTGTTC |
| | (SEQ ID NO: 29) | (SEQ ID NO: 30) |

Absolute quantification of Esr1 mRNA. Full-length Esr1 mRNA was generated by in vitro transcription from a 17 promoter present upstream of Esr1 construct (pcDNA-Esr1) using the MaxiScript T7/SP6 in vitro transcription kit (Catalog #AM1322, Thermo Fisher). Esr1 mRNA generated by in vitro transcription was purified using BioRad Aurum RNA isolation kit and reverse transcribed to cDNA using iScript cDNA synthesis kit. cDNA generated from this reaction was used to generate standards (7.5 ng-0.075 fg range) and the absolute amount of RNA present in the BMDM isolated from Esr1^{f/f} and Esr1^{f/f}; LysMCre mice was determined by plotting Ct values generated from BMDM cDNA to the standard cDNA.

sIRNA transfection. B16F10 (50,000 cells/mL) and YuMM5.2 (50,000 cells/mL) cells were transfected with either a scrambled siRNA (Catalog #AM4637, Thermo Fisher Scientific) or Esr1 (50 nM) siRNA using the Dharmafect 1 transfection agent according to the manufacturer's instruction. Cells were collected for downstream analysis after 48 hrs (RNA) or 72 hr post transfection (protein). The siRNA sequences used are listed below in TABLE 2.

TABLE 2
Sequences of siRNA.

| Gene ID | Sequences |
|---|---|
| siEsr1 #1 | AUAUUCAGAAUAGAUCAUGGG (SEQ ID NO: 31) |
| siEsr1 #2 | UGUGCUUCAACAUUCUCCCTC (SEQ ID NO: 32) |
| siEsr1 #3 | UGCUUAAUCACAAGAGGGCTT (SEQ ID NO: 33) |

Proliferation assay. For proliferation assays, B16F10 cells were plated in DMEM media (without phenol red) supplemented with 10% charcoal-stripped FBS. Cells were plated at a concentration of 1000 cells/well of a 96-well plate for 2 days in 200 μL of media. After 2 days, 50 μL of media was removed and replenished with 50 μL of fresh media containing 4× concentration of vehicle (DMSO), E2, or E2+ fulvestrant at stated concentrations. Cells were collected every 24 hr by discarding the media from the plates. Plates were frozen at −80° C. Frozen plates were thawed at room temperature and 100 μL of water was added to each plate to mediate cell lysis. Cell numbers were determined by the addition of 100 μL of (conc.) DNA dye Hoechst 333258 dye (Sigma Cat #94403) in TNE buffer (10 mM Tris, 2M NaCl and 1 mM EDTA) and the fluorescence was read at an excitation of 360 nm and an emission at 465 nm using a microplate reader.

Single cell isolation from tumors. Tumors were isolated, minced on a petri dish in media (DMEM+5% FBS), and then enzymatically digested by the addition of 100 μg/mL DNase I (D5025-150KU, Sigma-Aldrich) and 1 mg/mL collagenase (Collagenase A, Cat #10103586001, Sigma-Aldrich) for 30 mins-45 mins. For iBP models, isolated tumors were sliced into large chunks and subjected to mechanical digestion in a gentleMACS Dissociator for 30 seconds twice. Following this, tumors were digested with an enzyme cocktail containing DNase I, collagenase, and hyaluronidase (100 μg/mL) (H6254, Sigma-Aldrich) for 40 mins following a second round of mechanical digestion for 30 seconds (twice). The cells were then filtered through a 40 μm strainer to produce single cell suspensions and the enzymes were diluted by addition of additional media then spun down to remove media. Red blood cells were lysed with the addition of ACK lysis buffer (Cat #A1049201. ThermoFisher Scientific) for 4 mins at room temperature. Following red blood cell lysis, cells were washed with PBS before proceeding to flow cytometry staining or magnetic bead-based isolation.

Flow cytometry staining. Single cells suspensions (10⁶ cells in 50 μL) were incubated with Live/dead fixable dead cell stain in PBS for 10 mins at 4° C. Cells were spun down at 1000×g and were incubated with anti-CD16/32 (Catalog #14-0161-85, ThermoFisher Scientific) in flow buffer (10 gms BSA in 1 L PBS+sodium azide) for 15 mins. Following this, cells were stained with an antibody cocktail in Brilliant Stain Buffer (Cat #566349, ThermoFisher Scientific). The antibodies used are listed below in TABLE 3. For intracellular staining, cells were fixed and permeabilized using the eBioscience Foxp3 Transcription Factor Staining Buffer Set (Cat #00-5523-00, ThermoFisher Scientific) followed by intracellular staining with the desired antibody for 30 mins at 4° C. Multicolor flow cytometry was performed in BD Fortessa 16 color analyzer. The FACS results were

TABLE 3
Antibodies used for flow cytometry staining.

| | | | Catalog | Working |
|---|---|---|---|---|
| Reagents | Clone | Source | number | concentration |
| Live/Dead Staining dye | | Invitrogen | L34964 | 1:200 |
| BV650 anti mouse CD45 | 30-F11 | Biolegend | 103139 | 1:800 |
| PerCpCy5.5 anti mouse CD3 | 17A2 | BD | 560572 | 1:50 |
| AF647 anti human/mouse | GB11 | Biolegend | 515406 | 1:50 |
| Granzyme B | | | | |
| AF700 anti mouse/human | IM7 | Biolegend | 103026 | 1:100 |
| CD44 | | | | |
| APC-Cy7 anti hamster | H1.2F3 | Biolegend | 104526 | 1:100 |
| CD69 | | | | |
| BV650 anti rat CD8 | 53-6.7 | Biolegend | 100742 | 1:100 |
| BV785 anti rat CD4 | RM4-5 | Biolegend | 100552 | 1:100 |
| BV711 anti rat IFNg | XMG1.2 | Biolegend | 505835 | 1:50 |
| PE anti rat FOXP3 | FJK-16s | eBioscience | 12-5573-82 | 1:100 |
| BV510 anti mouse PD1 | 29F1A12 | Biolegend | 135241 | 1:100 |
| APC anti rat CD25 | PC61.5 | eBioscience | 17-0251-82 | 1:100 |
| PE anti rat CD11b | M1/70 | Biolegend | 101208 | 1:50 |
| AF488 anti mouse CD206 | C06C2 | Biolegend | 141710 | 1:100 |
| BUV496 anti rat CD24 | M1/69 | BD | 564664 | 1:100 |
| PerCPCy5.5 anti mouse | X54- | Biolegend | 139308 | 1:100 |
| monoclonal CD64 | 5/7.1 | | | |
| APC anti rat F4/80 | BM8 | Biolegend | 123116 | 1:100 |
| APC Cy7 anti hamster | HL3 | BD | 561241 | 1:50 |
| CD11c | | | | |
| PE-CY7 anti rat MHCII | M5/114- | eBioscience | 25-5321-82 | 1:600 |
| | 15.2 | | | |
| BV711 anti rat Ly6 C | HK1.4 | Biolegend | 128037 | 1:100 |
| BV786 anti mouse Ly6 G | 1A8 | Biolegend | 127645 | 1:100 |

In vitro bone marrow-derived macrophage differentiation. For this purpose, bone marrow cells were aseptically collected from 8-10 weeks old female C57BL/6J mice by crushing the femurs and tibias in PBS, 1% PBS and 2 mM EDTA. Cells were added to ACK buffer to lyse the red blood cells for 2 mins with intermediate vortexing. The solution was filtered through a 40 μm strainer to remove bone fragments. To differentiate bone marrow cells to macrophages the cells were plated in DMEM media (100%) or DMEM media (70%) and 30% tumor-conditioned media, supplemented with 10% heat-inactivated charcoal-stripped serum in the presence of 30 ng/mL MCSF (Cat #312-02, PeproTech). After 3 days cells were supplemented with 50% of respective fresh media. On day 6 the media was removed and replaced with fresh media. When the cells are fully differentiated to macrophages on day 7, they are treated overnight with either DMSO, E2 (1 nM) or E2+fulvestrant (100 nM). For polarization, cells were further treated with LIPS (100 ng/mL, Cat L2630, Sigma Aldrich) and IFNγ (20 ng/mL, Cat #315-05, PeproTech) (24 hr) for M1 polarization or IL4 (10 ng/mL, cat #214-14, PeproTech) for M2 polarization.

T cell proliferation assay and staining. CD3+ T cells were isolated from the spleens of C57BL/6J or P-mel mice with magnetic bead-based T cell isolation kit (Cat #19851, StemCell Technologies). T cells from naïve mice were stained with 5 μM CFSE (Cat #C34554, ThermoFisher Scientific) for 5 minutes in PBS+5% CFS with rapid vortexing following which the cells were washed twice with PBS. Stained T cells were then counted and plated in 96-well plates coated with anti-CD3 antibody (0.5 μg/mL, Cat #19851, ThermoFisher Scientific) anti-CD28 antibody (1 μg/mL, Cat #16-0281-86, Thermo Fisher Scientific) at desired density (250,000 T cells/50,000 CD11b or BMDM) in the presence of IL2 (50 ng/mL) (Cat #212-12, PeproTech). 72 hr after plating the cells were incubated with protein transport inhibitors brefeldin (Cat #00-4506-51, Thermo Fisher Scientific) and monensin (Cat #00-4505-51, Thermo Fisher Scientific) for 6 hr at a final concentration of 2 μM monensin and 3 μg/mL brefeldin after which they were collected and were processed for staining for flow cytometry.

Flow cytometry staining of grafted tumor Infiltrating T cells. For assessment of TIL function/cytotoxicity, T cells from established YuMM5.2 tumors were isolated after 14 days of tumor growth. For assessment of IFNγ and granzyme-B production by TILs, the isolated TILs were incubated with ionomycin (1 mg/mL) and phorbol myristate acetate (20 ng/mL) for 4 hr in the presence of protein transport inhibitors (brefeldin and monensin) at 37° C. and 5% CO₂. Surface and intracellular staining were performed as described in the section describing flow cytometry staining.

T cell depletion with α-CD8 antibody. For the purpose of CD8 depletion, C57BL/6J mice were injected with 200 μg/mouse of a rat anti-CD8 antibody (clone YTS169.4, cat #BE0017 BioXCell) or rat IgG2b anti-KLH isotype control (clone LTF2, cat #BE0090BioXCell) diluted in sterile PBS, 24 hr before tumor injection and every 4 days after tumor injection. The efficiency of CD8 depletion was analyzed at the end of the experiment by collecting cardiac blood and performing flow cytometry for T cell subpopulations.

Fulvestrant treatment Mice were injected with fulvestrant (Cat #HY-13636 MedChemExpress) 2 days after tumor injection at a dose of 25 mg/kg via intramuscular route. After the initial injection fulvestrant treatment was administered to the mice every 5 days. Corn oil (Cat #C0136, Spectrum Chemical MFG Corp) was used as vehicle for fulvestrant which was administered at the same frequency to all other animals.

Anti-PD1 tumor studies. Age matched C57BL/6J mice harboring B16F10 tumors were treated with α-PD1 (clone RMP-14, cat #0146 BioXCell) or rat IgG2a (clone 2A3, cat #BE0089 BioXCell) at 250 μg/mouse by i.p. injections starting at day 10 after tumor inoculation and every 3 days until the end point was reached.

Macrophage depletion by clodronate liposomes. C57BLJ6J mice were injected with B16F10 tumors into the right flank. 24 hr prior to tumor injection, mice were injected with 1 mg of clodronate liposomes (Liposoma B.V.) in 200 μL of PBS per mouse via intravenous route. Liposomes were further administered 7 days and 14 days after tumor injection. The efficiency of intratumoral macrophage depletion was verified by flow cytometry when the control group reached a tumor size of ˜1000 mm³.

Analysis of human correlates. Raw RNA-sequencing data were downloaded from the European Nucleotide Archive (ENA) accession number PRJEB23709, Gene Expression Omnibus (GEO) accession number GSE78220, and dbGAP accession number phs000452.v2.p1 (Gide et al., Cancer Cell 2019, 35, 238-255; Van Allen et al., Science 2015, 350, 207-211; Hugo et al., Cell 2016, 165, 35-44). Results were aligned and quantified relative to reference genome hg38 using a STAR-Salmon pipeline as previously described (Hollem et al., Cell 2019, 179, 1191-1206) and upper quartile normalized. Hematopoietic immune cell relative fractions were determined from RNA expression data using CIBERSORT (Newman et al., Nat. Methods 2015, 12, 453-457). Cell populations were determined using the LM22 signature from CIBERSORT using 100 permutations and disabling quantile normalization. Survival analyses were performed using the ‘survival’ package’ analysis with R−. Patient populations were partitioned using median expression values and compared using the log-rank test.

Statistics. Statistics were performed using GraphPad Prism 8.0 software, by either two-tailed Student's T test, one-way ANOVA or two-way ANOVA, as indicated in the brief description of the drawings. For both one-way and two-way ANOVA, post-test analysis was performed using Bonferroni's multiple correction. Number of replicates are provided in the brief description of the drawings. Level of significance was determined to be p<0.05.

### Example 2

### Decreased M1/M2 Tumor Associated Macrophage (TAM) Ratio Compromises the Benefit of ICB Therapy in Melanoma Patients

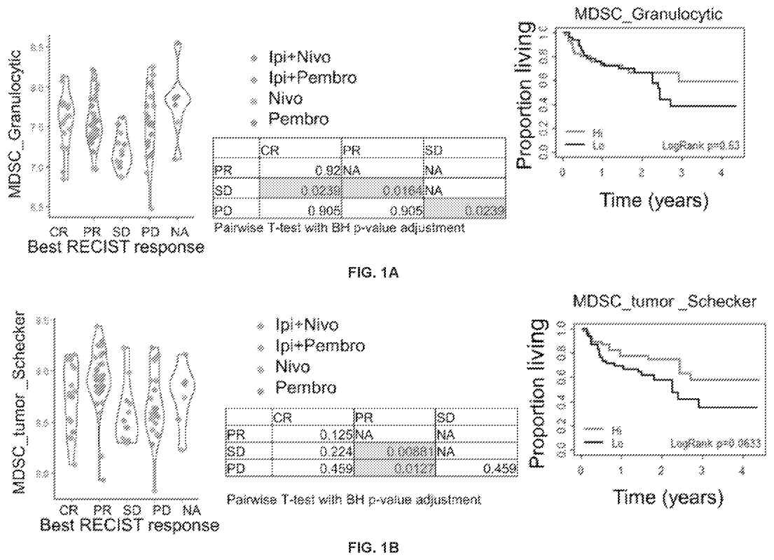

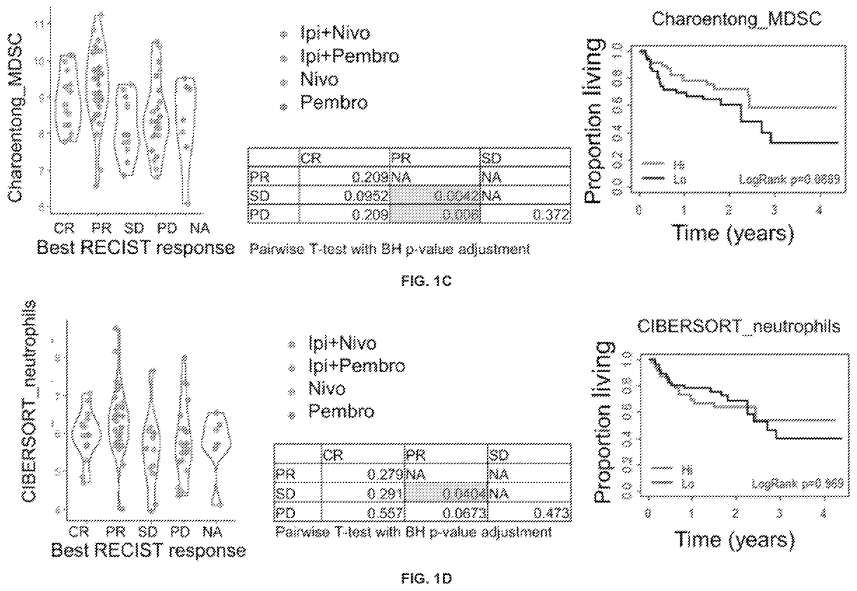

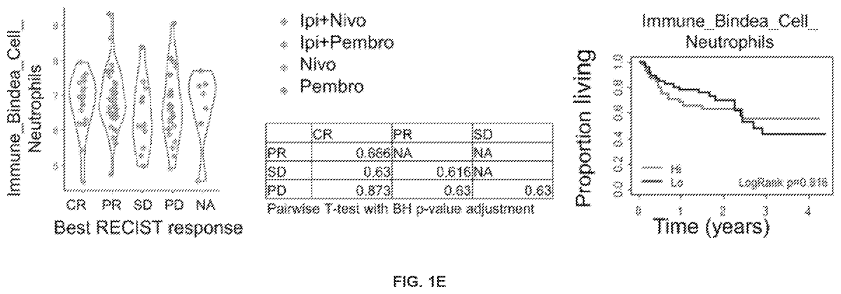

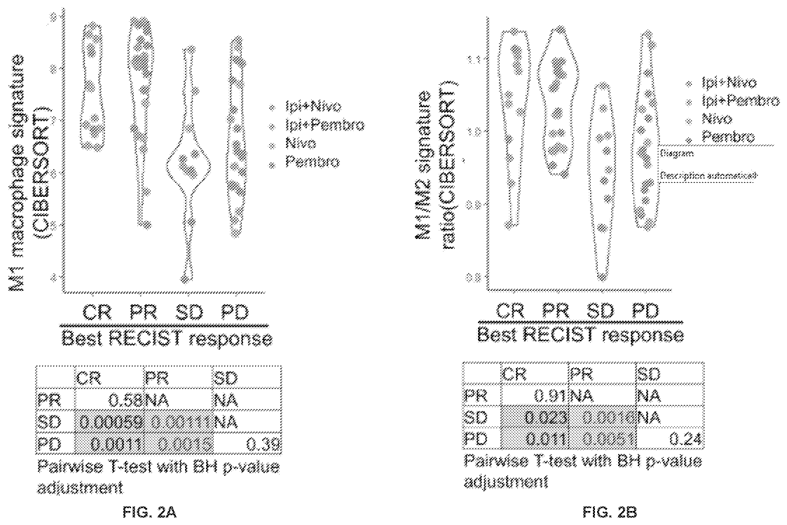

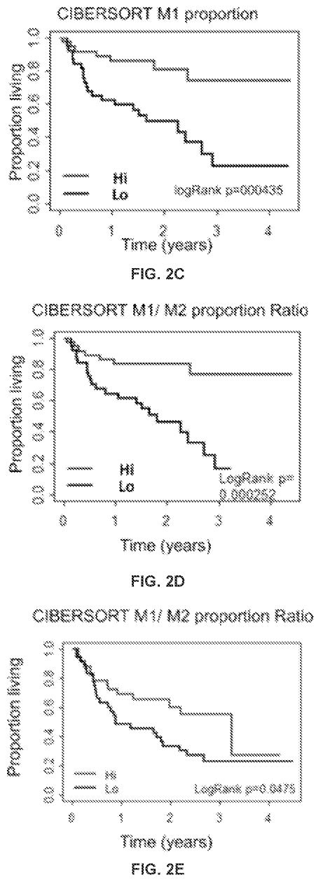

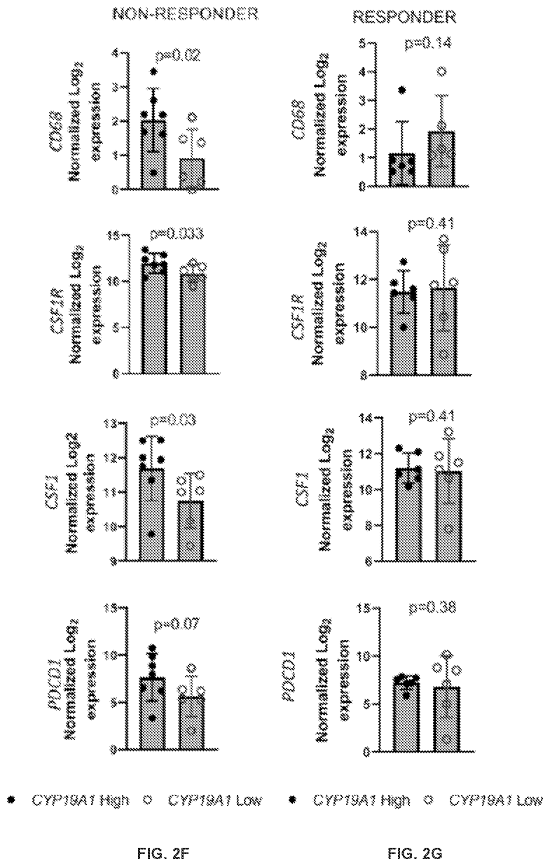

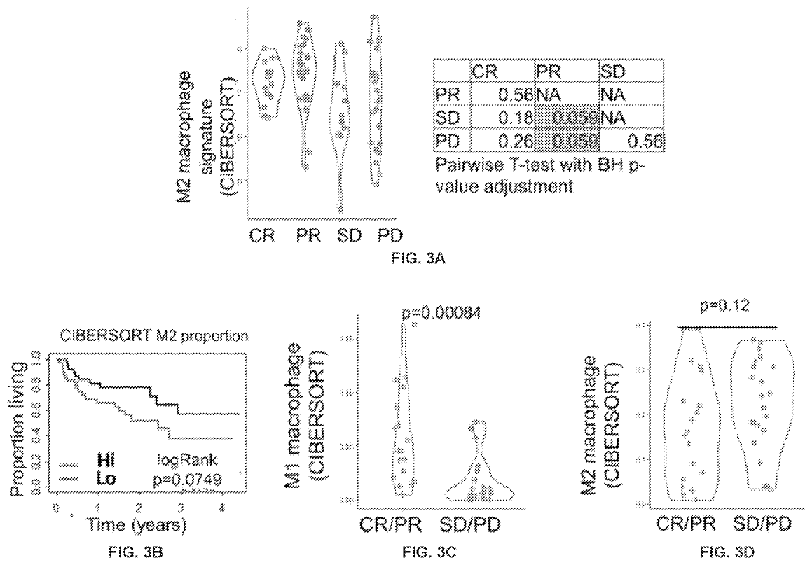

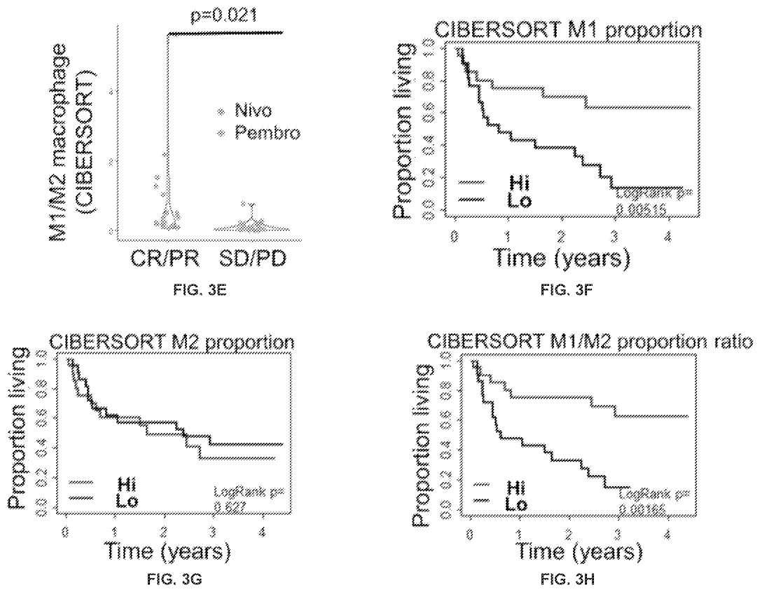

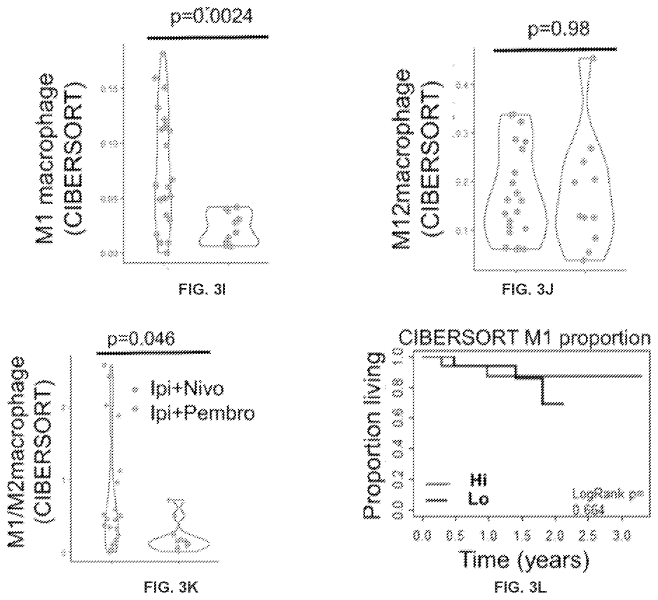

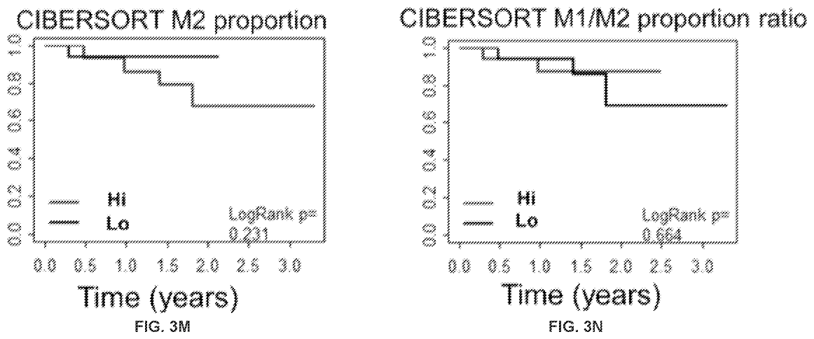

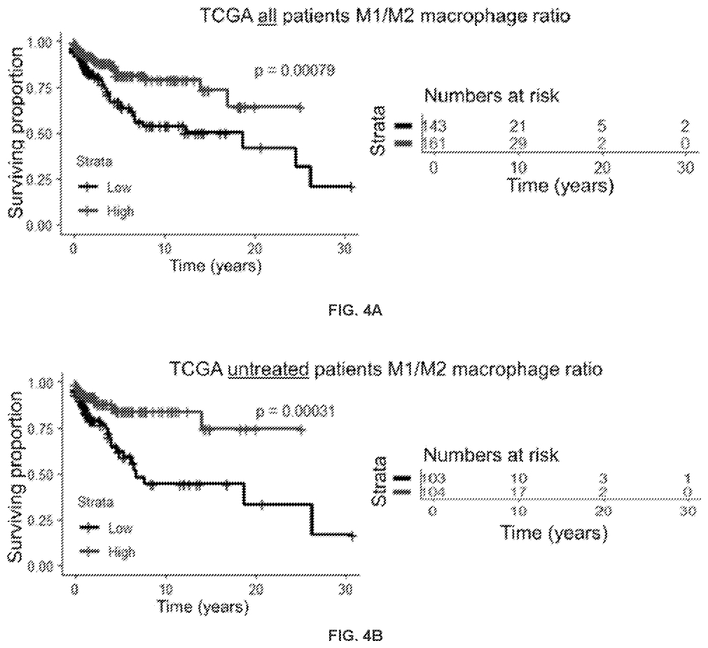

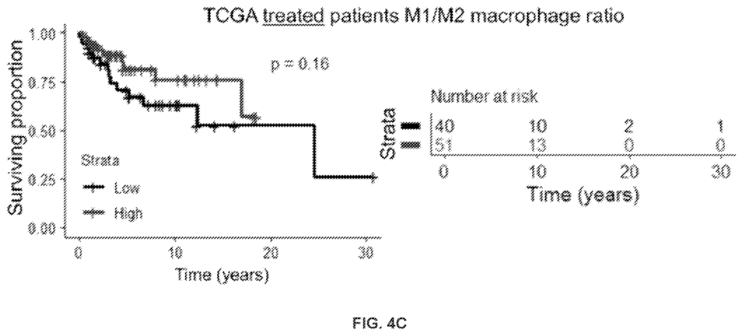

Myeloid cell infiltration has been associated with poor outcomes in multiple cancer types. However, the extent to which tumor infiltrating myeloid cells influence response to immunotherapy in melanoma patients has not been explored. To address this issue, potential correlations between the number and characteristics of tumor infiltrating myeloid cells and patient's response to ICB were evaluated using published transcriptomic datasets from melanoma patients who had received standard of care immune checkpoint blockade. The predominant suppressive myeloid cells in the tumor microenvironment are myeloid derived suppressor cells (MDSC) and tumor associated macrophages (TAMs). To address whether MDSCs play a role in predicting patient response to ICB, a validated MDSC gene signature was used to analyze transcriptomic data from melanoma patients who have received α-PD1 (Nivolumab or Pembrolizumab) or α-CTLA4 (Ipilimumab) either alone or in combination. As shown in FIGS. 1A-1E, MDSC signatures were not predictive of patient's response to ICB or survival. In contrast, signatures from CIBERSORT, that read on the polarization state of TAMs are useful in predicting ICB response in the same datasets. Notably, enrichment of the M1 gene signature in tumors was associated with better responses (increased number of complete responders (CRs) and partial responders (PRs)) when compared to patients with stable disease (SD) or progressive disease (PD) (FIG. 2A). A similar trend in patient prognosis was also observed when patients were parsed as a function of high vs. low intratumoral M1/M2 macrophage ratio (FIG. 2B). Enrichment of the M2 signature alone did not correlate with patient prognosis (FIG. 3A). Using the same dataset, it was also addressed whether the macrophage gene signature is associated with overall survival in melanoma patients receiving immunotherapies. Similar to what was observed with patient prognosis (FIGS. 2A-2B), an enrichment of either the M1 gene signature or the M1/M2 ratio gene signature, but not enrichment of the M2 signature, was associated with better overall survival (FIGS. 2C-2D and FIG. 3B). Interestingly, a positive association between the enrichment of an M1 gene signature, or the ratio of M1/M2 gene signature, with patient prognosis and survival was also noted when the patients were parsed for those who received α-PD1 monotherapy alone (FIGS. 3C-3H), while those patients who received dual therapy showed a non-significant trend in this association (FIGS. 31-3N). Additionally, an increase in intratumoral M1/M2 ratio predicted better survival in melanoma patients in the TCGA SKCM dataset (FIGS. 4A-4C). The prognostic utility of assessing the intratumoral M1/M2 macrophage ratio was confirmed in independent datasets derived from melanoma patients treated with immunotherapy (FIG. 2E). It has been reported in several studies that gender influences patient response to immunotherapy in melanoma, with females receiving a lesser degree of benefit from ICB than males. Motivated by these observations and previous studies demonstrating that female steroid hormone estrogens (E2) affect macrophage differentiation and polarization, it was hypothesized that estrogens may modulate the tumor microenvironment to promote immunotherapy resistance. It was of significance, therefore, that it was observed that increased expression of CYP19A1, the enzyme that controls the rate-limiting step in estrogen biosynthesis, was correlated with increased TAM accumulation in ICB non-responsive melanoma patients (FIGS. 2F-2G). Importantly, stratification of patients based on tumor expression of CYP19A1 mRNA revealed its elevated expression to be associated with the expression of the macrophage markers CD68, CSF1, CSF1R, and the T cell exhaustion marker PDCD1 (FIG. 2F) in non-responders whereas no such associations were identified in responder patient populations (FIG. 2G). These results suggested that E2 may be causally involved in the establishment of an immune suppressive tumor microenvironment through modulating TAM biology; a hypothesis that was then tested experimentally.

### Example 3

### E2 Promotes Melanoma Tumor Growth

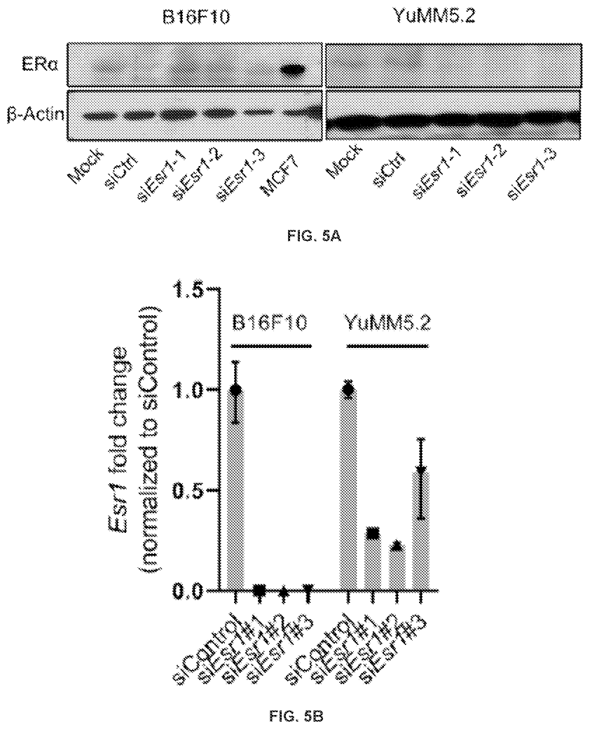

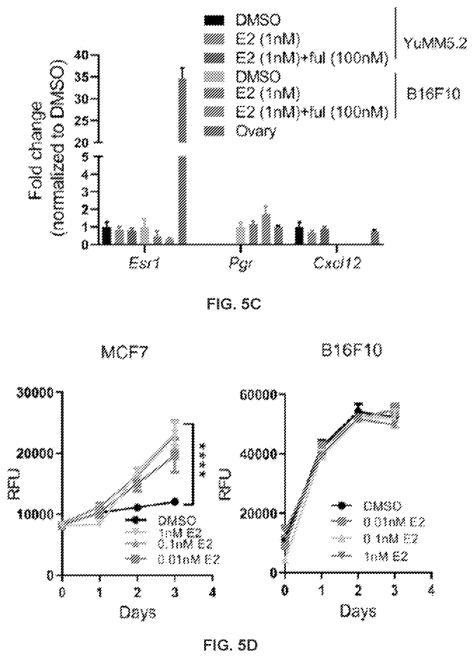

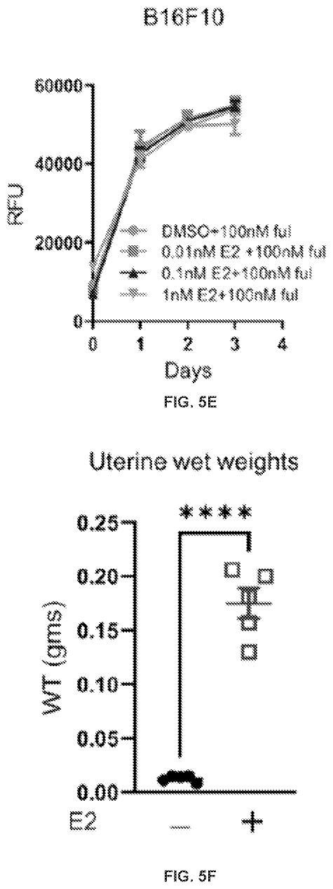

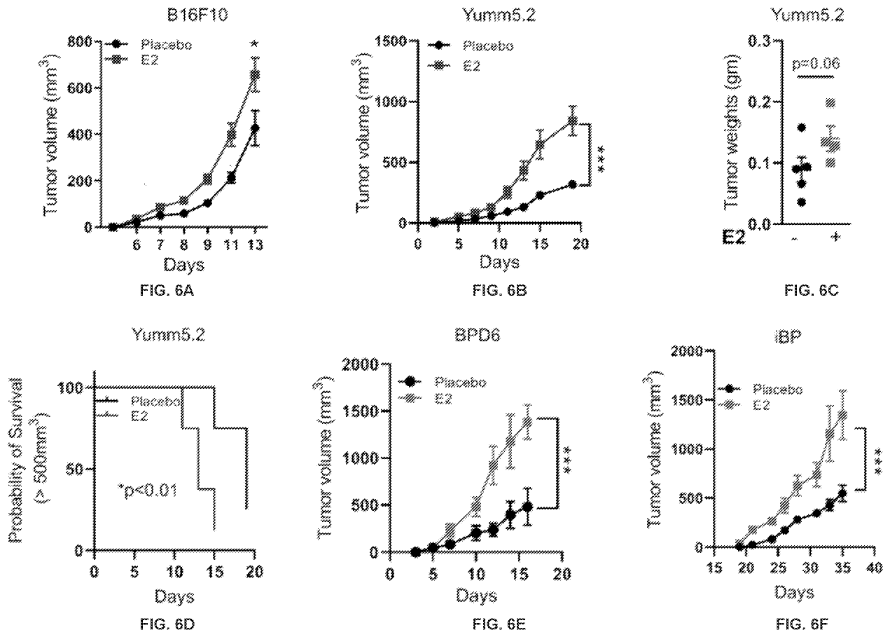

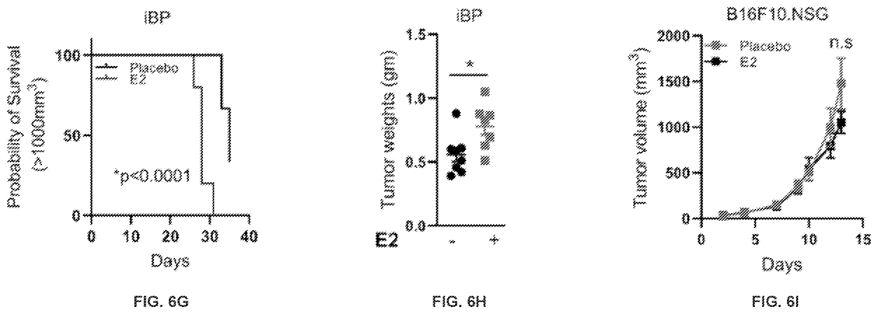

The results of studies addressing whether ERs are expressed within melanoma cells/tumors are equivocal. While some studies have demonstrated low expression of ERα and ERS in human melanoma tumors by immunohistochemical staining (IHC), the functionality of these receptors within tumor cells is unknown. Thus, the expression of ERα in B16F10 and YuMM5.2 mouse melanoma cells was evaluated following siRNA-mediated knockdown of Esr1. ERα+MCF7 cells were used as a positive control for ERα expression. Weak ERα protein was detected in YuMM5.2 cells and this was depleted upon siRNA treatment (FIGS. 5A-5B). By immunoblotting, ERα protein in B16F10 cells was unable to be detected (a band migrating at approximately the same size as ERα was not depleted upon siRNA treatment despite a significant reduction of ERα mRNA (expressed at very low level)). Regardless, treatment of either cell with E2 did not lead to changes in the expression of classical ER target genes (Pgr and Cxc112) (FIG. 5C) nor did it support proliferation (FIGS. 5D-5E). Collectively, these data validated the use of these cell models to study the cancer cell extrinsic actions of estrogens/ER modulators on the pathobiology of melanoma. To this end, B16F10, YuMM5.2, or BPD6 melanoma cells were injected subcutaneously into ovariectomized syngeneic mice supplemented with either placebo or E2 pellets (0.01 mg/60 days continuous release). As expected, E2 administration resulted in an increase in uterine wet weights in the ovariectomized mice (FIG. 5F). As shown in FIGS. 6A-6E, E2 treatment significantly increased tumor growth in all three syngeneic models compared to placebo control mice. To further validate these observations in a more clinically relevant system, an autochthonous mouse model was used in which tumor growth was driven by concomitant conditional activation of B-Raf^{V600E} and homozygous deletion of Pten in melanocytes (Brat^{tm1Mmcm}, Pten^{f/f}; mTyr-CreERT2, heretofore referred as iBP). This mouse model faithfully resembles human melanomas harboring BRAF and PTEN mutations. Similar to the syngeneic models, administration of E2 in ovariectomized mice accelerated tumor growth in the iBP model compared to the placebo counterparts (FIGS. 6F-6H). The slower tumor growth kinetics that were imparted by ovariectomy disappeared when B16F10 cell derived tumors were grown in NOD.Cg-Prkdcs^{scid} Il2rg^{tm1Wjl}/SzJ (NSG) mice (FIG. 6I), suggesting that the actions of E2 on tumor growth were likely mediated by an immune cell(s).

### Example 4

### E2 Regulates the Function of Tumor-Associated Myeloid Cells

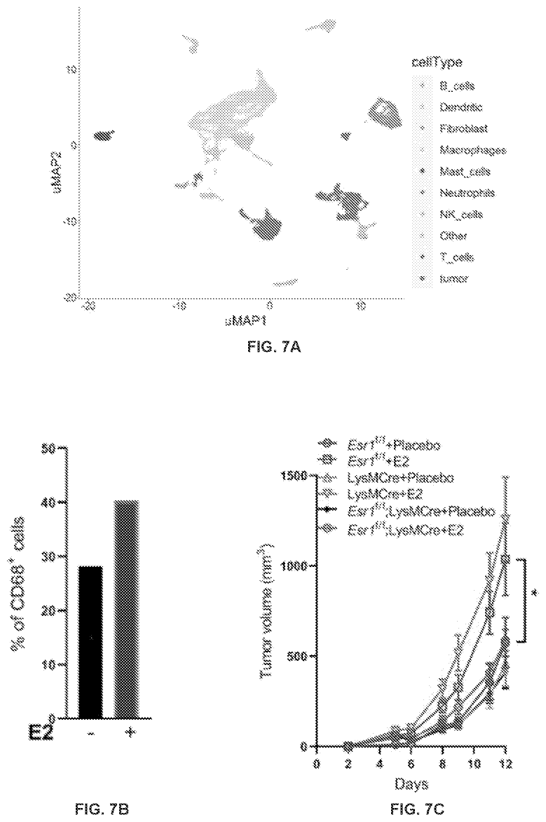

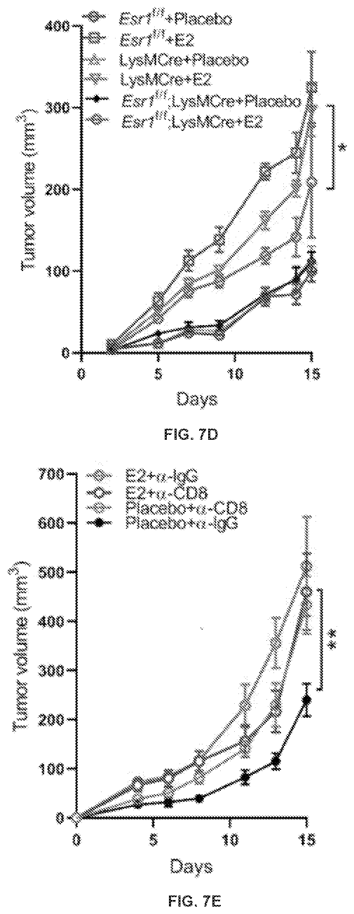

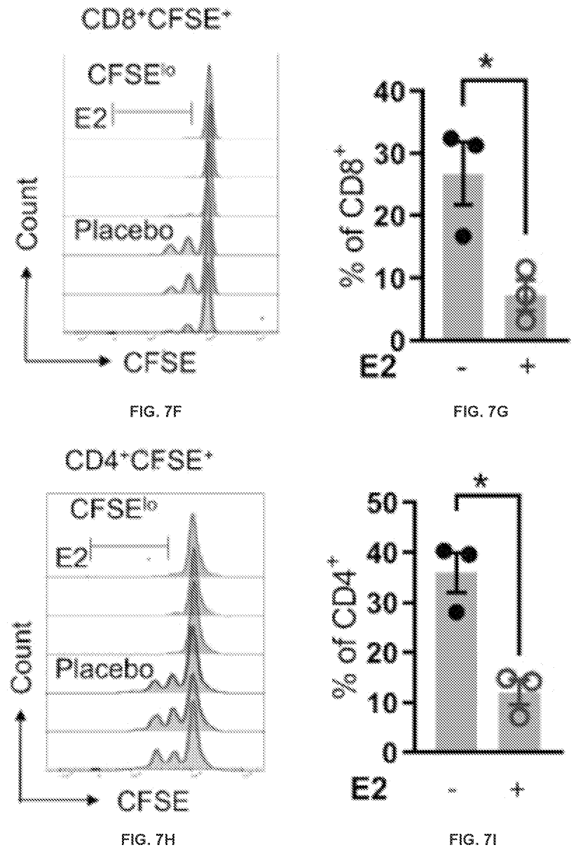

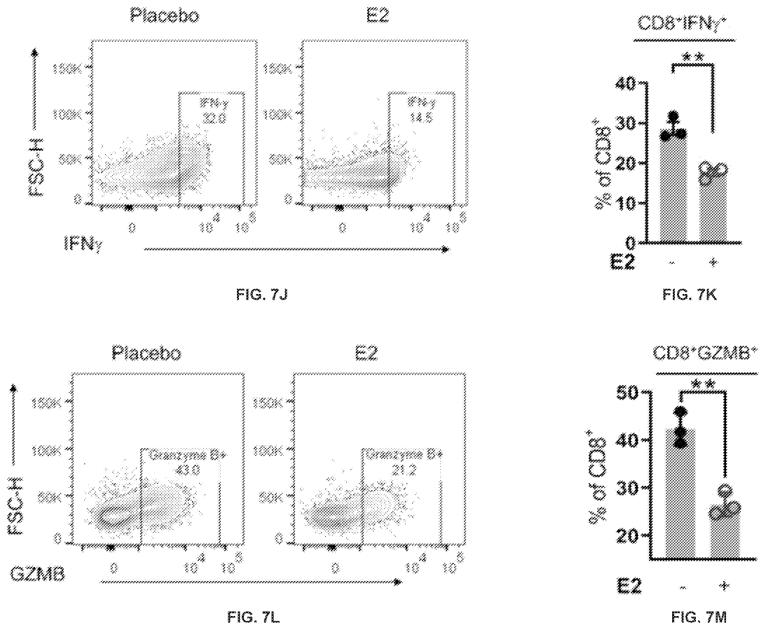

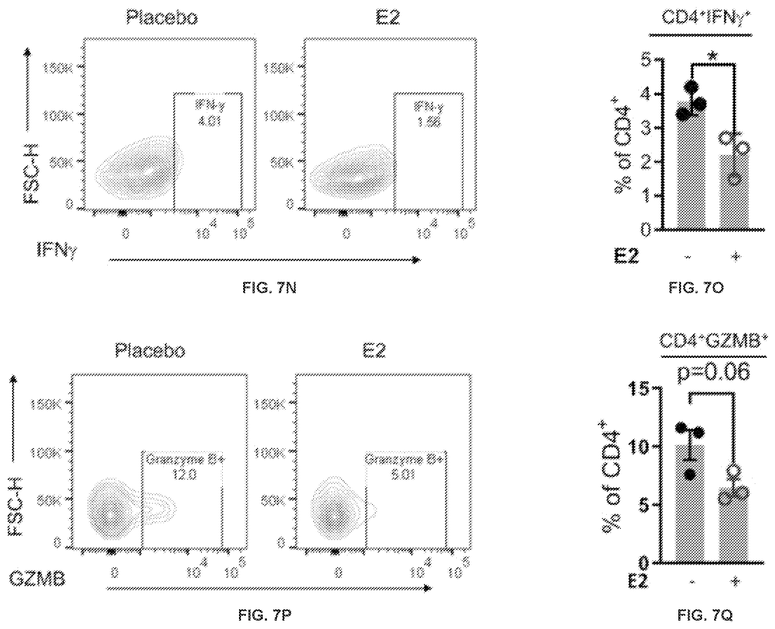

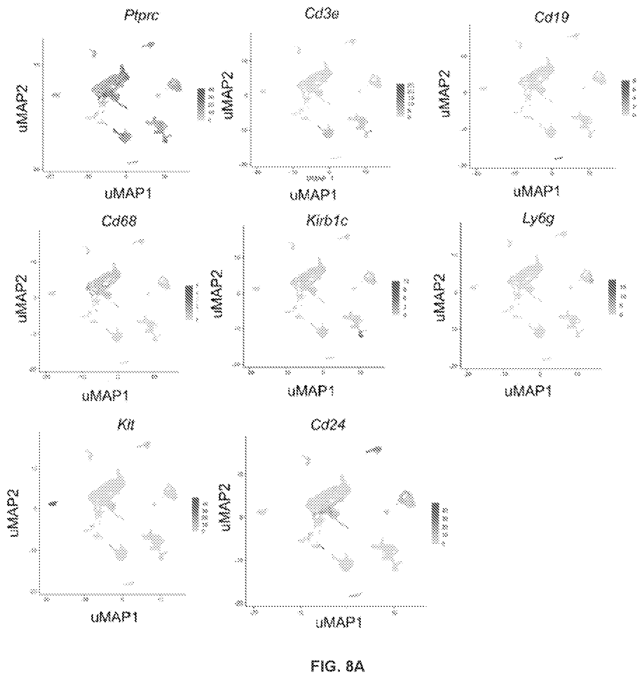

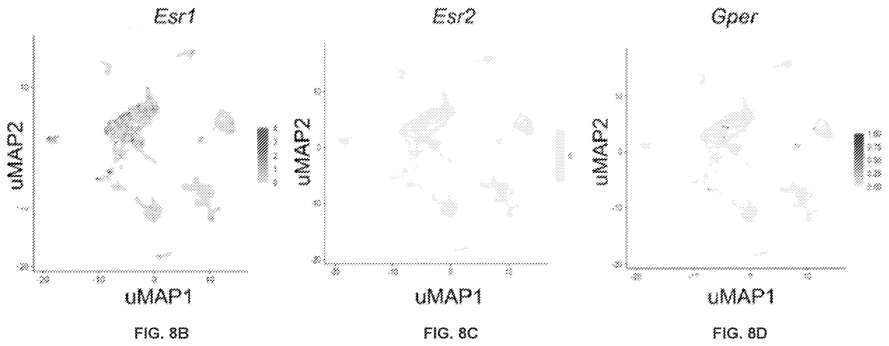

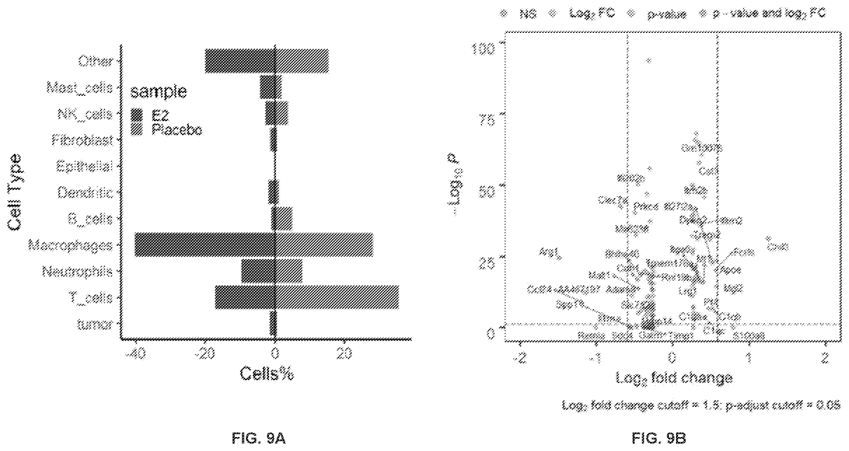

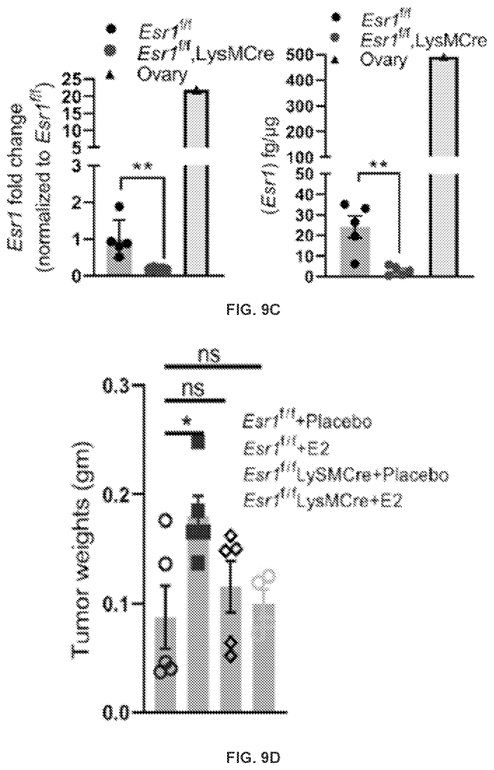

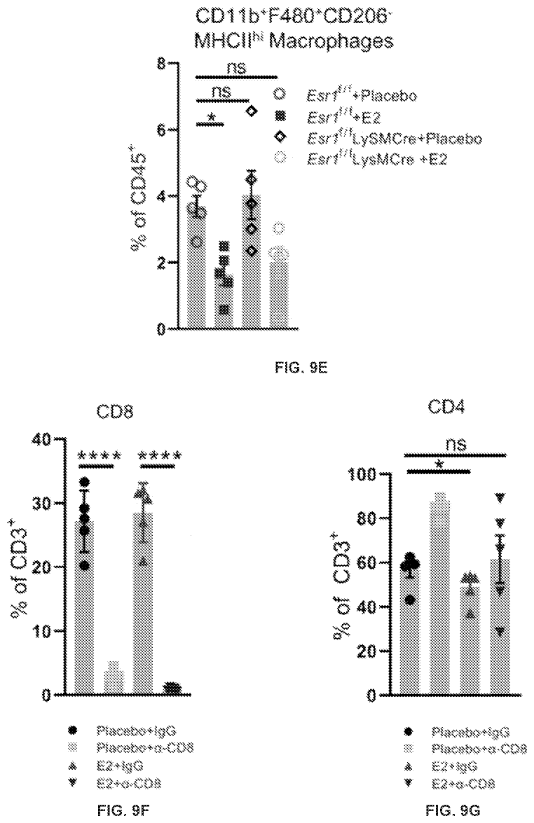

To determine how E2 treatment affects the tumor immune microenvironment, single cell RNA sequencing (scRNA seq) analysis was performed of tumor infiltrating immune cells isolated from iBP tumors treated with either placebo or E2. Unsupervised clustering analysis using uniform manifold approximation and projection (UMAP) revealed global differences in tumor infiltrating immune cells when comparing placebo and E2 treatments and identified clusters of immune cells that have unique transcriptional profiles. Comparison of cell type signature(s) with the Immgen database and known cell type markers (TABLE 4) resulted in the identification of 9 macrophage/myeloid clusters, 10 lymphoid clusters, 2 neutrophil clusters, 2 DC clusters and one B cell, NK cell, and mast cell cluster (FIG. 7A and FIG. 8A). Analysis of the scRNA seq dataset also revealed that the majority of Esr1 transcripts were expressed in cells within the myeloid lineage, while the expression of Esr2 and Gperwere minimal to undetectable (FIGS. 8B-8D). Differences in the immune cell repertoires from placebo and E2 treated tumors were also evident (FIG. 9A). Notably, E2 treatment led to the expansion and significant changes in gene expression in the CD68⁺ monocytes/TAMs clusters (FIG. 7B and FIG. 9B). To determine the functionality of ER signaling in the monocyte/TAM cluster, ERα was genetically depleted in myeloid cells using a lysozyme-driven Cre-recombinase (Esr1^{f/f}; LysMCre) to establish its role(s) in tumor responses to E2. ERα depletion in the myeloid lineage was confirmed in bone marrow derived macrophages (BMDM) isolated from Esr1^{f/f}; LysMCre and littermate Esr1^{f/f} controls (FIG. 9C). Subsequently, 8-week old Esr1m; LysMCre, and littermate control (Esr1^{f/f} and LysMCre) mice, were used to evaluate syngeneic tumor growth in the B16F10 and Yumm5.2 models, in the presence or absence of E2. The growth of B16F10 and YuMM5.2 tumors increased in response to E2 in Esr1^{f/f} and LysMCre mice but this was not evident in Esr1^{f/f}; LysMCre mice (FIGS. 7C-7D and FIG. 9D). Analysis by flow cytometry of tumor infiltrating immune cells revealed a decrease in M1 (proinflammatory macrophages) in E2 treated Esr1^{f/f} but not Esr1^{f/f}; LysMCre animals (FIG. 9E). Myeloid cells can often manifest their actions by modulating other cell types in the TME either by facilitating the release of cytokines and/or by blunting antigen presentation to the adaptive immune cells. To understand whether T cells play a functional role in E2 induced tumor growth, CD8⁺ T cells were depleted with an α-CD8 antibody in mice engrafted with YuMM5.2 tumor cells in the presence or absence of E2. The efficacy of the CD8⁺ T cell depletion was confirmed by flow cytometry analysis (FIGS. 9F-9G). Antibody-mediated acute depletion of CD8⁺ T cells reversed the protective effects of ovariectomy on YuMM5.2 tumor growth but did not accelerate tumor growth in E2 treated mice (FIG. 7E). These results suggested the functional involvement of CD8+ T cells in E2-mediated tumor growth.

TABLE 4
Gene sets for cluster determination.

| Geneset name | Genes used for identification of cell types |
|---|---|
| IMMUNE_Bindea_Cell_aDC_{—} | Ido1, Oas3, Ccl1, Ebi3, Lamp3 |
| Median_Immunity.2013_{—} | |
| PMID.24138885 | |
| IMMUNE_Bindea_Cell_{—} | Tnfrsf17, Blk, Cd19, Ms4a1, Cd72, Cr2, Coch, Dtnb, Gldc, |
| Bcells_Median_Immunity.2013_{—} | Gng7, H2-Ob, H2-Aa, Mef2c, Abcb4, Pnoc, Scn3a, |
| PMID.24138885 | Slc15a2, Spib, Tcl1, Ccr9, Blnk, Bcl11a, Qrsl1, Mical3, |
| | Bach2, Osbpl10 |
| IMMUNE_Bindea_Cell_DC_{—} | Hsd11b1, Npr1, Ccl2, Ccl17, Ccl22, Ppfibp2, Cd209e |
| Median_Immunity.2013_{—} | |
| PMID.24138885 | |
| IMMUNE_Bindea_Cell_iDC_{—} | Blvrb, Csf1r, Ctns, F13a1, Fabp4, Fzd2, Guca1a, Tacstd2, |
| Median_Immunity.2013_{—} | Mmp12, Pparg, Prep, Rap1gap, Pdxk, Ch25h, Abcg2, |
| PMID.24138885 | Hs3st2, Clec10a, Vash1, Slc7a8, Syt17, Nudt9, Card9, |
| | Ms4a6d, Slc26a6, Dcstamp, Lman21 |
| IMMUNE_Bindea_Cell_Macrophages_{—} | Apoe, Bcat1, Scarb2, Cd68, Chil1, Chit1, Col8a2, Cybb, |
| Median_Immunity.2013_{—} | Emp1, Fdx1, Gpc4, Fn1, Gm2a, Me1, Msr1, Ptgds, Scg5, |
| PMID.24138885 | Sult1c2, Marco, Cd84, Cd163, Atg7, Clec5a, Rai14, |
| | Pcolce2, Ms4a4a, Dnase2b, Colec12, Sgms1, Ctsk |
| IMMUNE_Bindea_Cell_Mast | Adcyap1, Nr0b1, Calb2, Cma1, Cpa3, Ctsg, Elane, |
| cells_Median_Immunity.2013_{—} | Ms4a2, Gata2, Hdc, Hpgd, Kit, Vwa5a, Mpo, Prg2, Ptgs1, |
| PMID.24138885 | Slc18a2, Tal1, Tpsb2, Scg2, Abcc4, Hpgds, Slc24a3, |
| | Ppm1h, Tpsb2, Miph, Maob |
| IMMUNE_Bindea_Cell_Neutrophils_{—} | Alpl, Bst1, Csf3r, Fcgr4, Fpr1, Fpr2, Cxcr1, Cxcr2, Kcnj15, |
| Median_Immunity.2013_{—} | Cyp4f13, Mme, Pde4b, Slc22a4, Dysf, H2bc8, Mgam, |
| PMID.24138885 | Creb5, Tecpr2, Hpse, Cd93, G0s2, Sic25a37, Cpped1, |
| | Crispld2 |
| IMMUNE_Bindea_Cell_{—} | Adarb1, Aldh1b1, Apbb2, Bcl2, Cdc5l, Gnas, Igfbp5, |
| NKcells_Median_Immunity.2013_{—} | Psmd4, Xcl1, Spn, Tbxa2r, Zfp13, Pdlim4, Fgf18, |
| PMID.24138885 | Mcm3ap, Ncr1, Mrc2, Ldb3, Mapre3, Kank2, Fzr1, Trpv6, |
| | Ppp4r3a, Prx, Tinagl1, Atl2, Slc30a5, Tctn2, Sgms1 |
| IMMUNE_Bindea_Cell_pDC_{—} | II3ra |
| Median_Immunity.2013_{—} | |
| PMID.24138885 | |
| IMMUNE_Bindea_Cell_{—} | Cd2, Cd3d, Cd3e, Cd3g, Cd6, Cd28, Lck, Sh2d1a, Prkcq, |
| Tcells_Median_Immunity.2013_{—} | Skap1, Itm2a, Cd96, Trat1, Bcl11b, Ncald |
| PMID.24138885 | |
| IMMUNE_Bindea_Cell_T | Cd28, Lrba, Aff2, Nap1l4, Rpa1, Ube2l3, Slc25a12, Itm2a, |
| helper | Sec24c, Batf, Yme1l1, Srsf10, Asf1a, Icos, Phf10, |
| cells_Median_Immunity.2013 | Nup107, Fam111a, Ddx50, Bora, Fryl |
| PMID.24138885 | |
| B-cell_ASH | Vpreb3, Tnfrsf17, Tcl1, Spib, Rnase6, Rasgrp3, Ralgps2, |
| | Pnoc, P2ry14, P2rx5, Ms4a1, H2-Aa, H2-Ob, Hhex, Gng7, |
| | Fcgr2b, Fcer2a, Eaf2, Cxcr5, Cr2, Cd79b, Cd79a, Cd72, |
| | Cd40, Cd37, Cd22, Cd19, Cd180, Blk, Bcl7a, Bank1, |
| | Bach2, Alox5, Adam28, Abcb4 |
| B-cell_PEROU | Tnfrsf17, Tnfrsf13b, Pou2af1, Pax5, Ms4a1, Fcrla, Fcrl5, |
| | Fcrl1, Fcer2a, Cxcr5, Cd79b, Cd79a, Cd19, Bank1 |
| B-cell_IMMGEN | Vpreb3, Tnfrsf17, Tnfrsf13b, Tcl1, Tcf4, Syk, Spib, |
| | Rnase6, Rasgrp3, Ralgps2, Pou2af1, Pnoc, Pax5, P2ry14, |
| | P2rx5, Ms4a1, H2-Ea-ps, H2-Ab1, H2-Aa, H2-Ob, Hhex, |
| | Gng7, Gga2, Fcrla, Fcrl5, Fcrl1, Fcgr2b, Fcer2a, Ebf1, |
| | Eaf2, Cxcr5, Cr2, Cd79b, Cd79a, Cd74, Cd72, Cd40, |
| | Cd37, Cd22, Cd19, Cd180, Blnk, Blk, Bcl7a, Bcl11a, |
| | Bank1, Bach2, Alox5, Aff3, Adam28, Abcb4 |
| B-cell_PURVESH | Vpreb3, Tnfrsf17, Tcl1, Tcf4, Spib, Pou2af1, Pnoc, Pax5, |
| | P2rx5, H2-Eaps, H2-Ab1, H2-Aa, Fcrla, Fcer2a, Ebf1, |
| | Cd79a, Cd72, Cd40, Cd22, Cd19, Blnk, Blk, Bcl11a, |
| | Bank1 |
| T-cell_ASH | Zap70, Ubash3a, Trat1, Tnfrsf4, Tnfrsf14, St8sia1, Sit1, |
| | Sh2d1a, Ptgir, Ptger2, Prf1, Pdcd1, Nkg7, Map4k1, Ly9, |
| | Lta, Lef1, Lck, Lat, Klrg1, Klrd1, Klrb1a, Itk, Il7r, Il2ra, Il21, |
| | Il18rap, Icos, Gzmm, Gzmd, Gzma, Gpr171, Gfi1, Foxp3, |
| | Flt3I, Dusp2, Dsc1, Dgka, Cxcr6, Ctsw, Ctla4, Cst7, |
| | Crtam, Cd96, Cd8a, Cd7, Cd6, Cd5, Cd40lg, Cd4, Cd3g, |
| | Cd3e, Cd3d, Cd300a, Cd28, Cd247, Cd244a, Cd2, |
| | Cd160, Ccr7, Ccr5, Ccnd2, Ccl5, Ccl4, Bcl2a1a, Bcl11b, |
| | Ankrd55, Acap1 |
| T-cell_PEROU | Zap70, Ubash3a, Trat1, Tigit, Themis, Tbx21, Spock2, |
| | Slamf1, Sla2, Sit1, Sh2d1a, Samd3, Prf1, Pdcd1, Nkg7, |
| | Map4k1, Ly9, Lta, Lck, Lat, Itk, Icos, Gzmm, Gzmk, Gzma, |
| | Gpr171, Gfi1, Cxcr6, Cxcr3, Ctla4, Cst7, Crtam, Cd96, |
| | Cd8a, Cd7, Cd6, Cd5, Cd40lg, Cd3g, Cd3e, Cd3d, Cd27, |
| | Cd247, Cd2, Ccr2, Ccl5, Acap1 |
| T-cell_IMMGEN | Zap70, Ubash3a, Trat1, Tnfrsf4, Tnfrst25, Tnfrsf14, Tigit, |
| | Themis, Tbx21, St8sia1, Spock2, Slamf1, Sla2, Sh2d1a, |
| | Samd3, Ptgir, Ptger2, Prf1, Pdcd1, Nr3c2, Nosip, Nkg7, |
| | Mal, Lrrn3, Lef1, Ldlrap1, Lck, Lat, Klrg1, Klrd1, Klrb1a, |
| | Itk, Il7r, Il2ra, Il21, Il18rap, Icos, Gzmk, Gzmd, Gzma, |
| | Gpr171, Gfi1, Foxp3, Flt3l, Fbln5, Ephx2, Ephb6, Dusp2, |
| | Dsc1, Dgka, Cxcr6, Cxcr3, Ctsw, Ctla4, Cst7, Crtam, Cish, |
| | Cdr2, Cd96, Cd8a, Cd7, Cd6, Cd5, Cd40lg, Cd4, Cd3g, |
| | Cd3e, Cd3d, Cd300a, Cd28, Cd27, Cd247, Cd244a, Cd2, |
| | Cd160, Ccr7, Ccr5, Ccr4, Ccr2, Ccnd2, Ccl5, Ccl4, |
| | Bcl2a1a, Bcl11b, Ankrd55 |
| T-cell PURVESH | Ubash3a, Tnfrsf25, Themis, Nr3c2, Nosip, Mal, Lrrn3, |
| | Lef1, Ldlrap1, Il7r, Icos, Fbln5, Ephx2, Ephb6, Cish, Cdr2, |
| | Cd6, Cd5, Cd40lg, Cd3g, Cd3e, Ccr4 |

To define the extent to which E2 treated myeloid cells affect T cell functionality, CD11b⁺ myeloid cells were isolated from iBP tumors treated either with placebo or E2. These cells were then co-incubated with CD3⁺ T cells isolated from the spleens of non-tumor bearing Pmel mice (Thy1^{a}/Cy Tg(TcraTcrb)8Rest/J) for 72 hr. iBP tumors express gp100 (Pmel) that can be processed and presented by professional antigen presenting cells to T cells that are specific to the antigen (gp100). Prior to coincubation, T cells were stained with the Carboxyfluorescein succinimidyl ester (CFSE) dye and activated in the presence of sub-optimal CD3/CD28. As assessed by CFSE dye dilution, it was apparent that T cell (both CD4⁺ and CD8⁺) proliferation was significantly inhibited by co-incubation with myeloid cells isolated from tumors of E2 treated mice as compared to those T cells that were incubated with myeloid cells isolated from placebo treated mice (FIGS. 7F-7I). Additionally, myeloid cells from E2 treated mice also affected the cytotoxic capability of both CD8⁺ and CD4⁺ T cells as demonstrated by decreased expression of IFNγ (FIGS. 7J-7K and FIGS. 7N-7O) and granzyme B (GZMB) (FIGS. 7L-7M and FIGS. 7P-7Q). Taken together, these observations suggest that the ERα/E2 axis increases the immunosuppressive activities of tumor-infiltrating myeloid cells. In this experiment, the phenotypic characteristics of the isolated myeloid cells were not defined, i.e., bone marrow derived vs resident macrophages. However, in subsequent experiments it was determined that the suppressive effects of E2 were likely mediated by macrophages that differentiate from monocytes recruited to the tumor from the bone marrow.

### Example 5

E2 Promotes the Accumulation of Immune-Suppressive TAMs within the Tumor Microenvironment

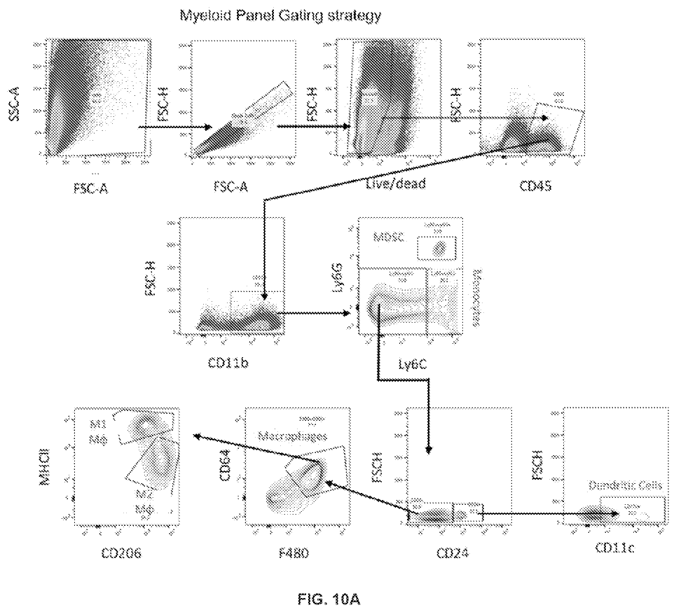

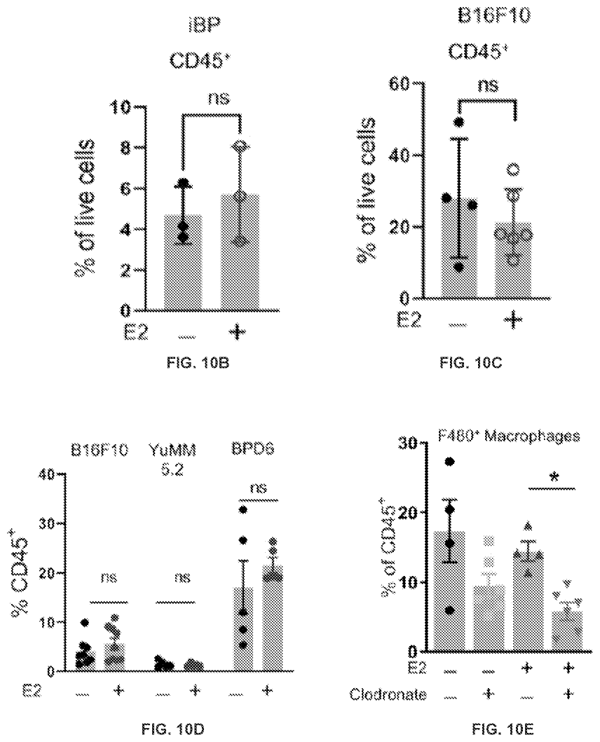

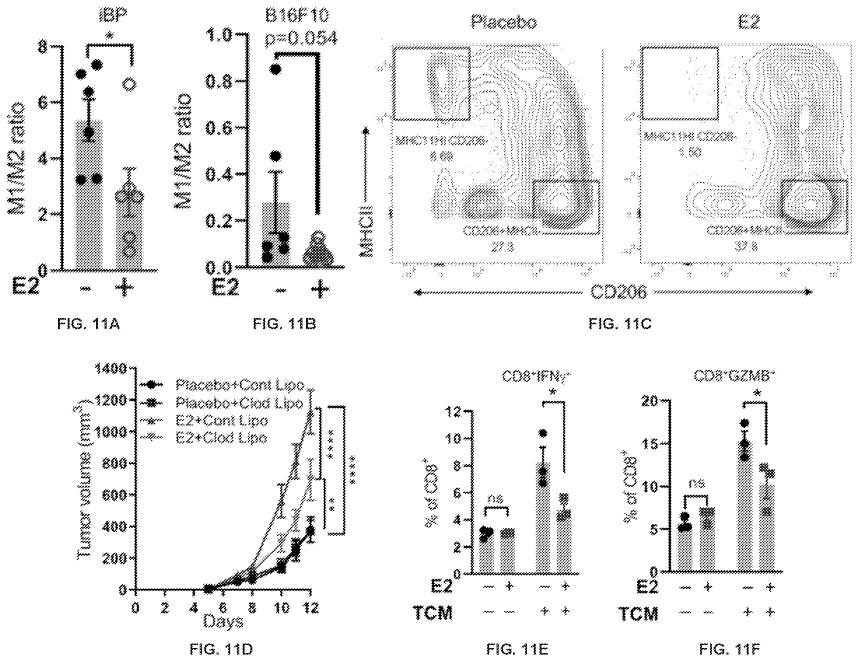

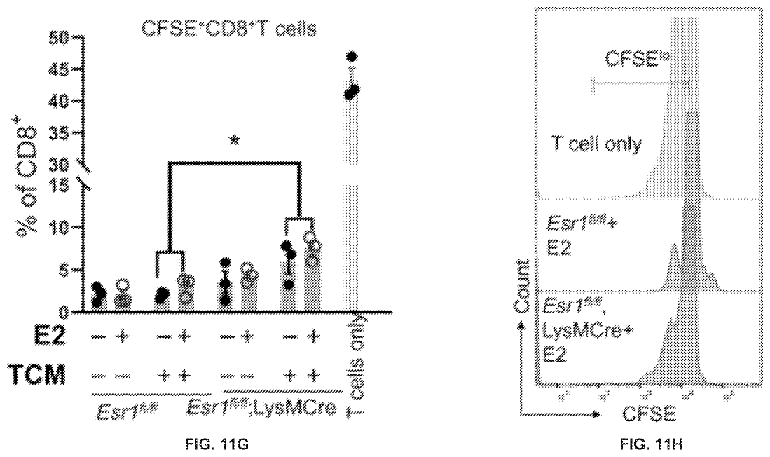

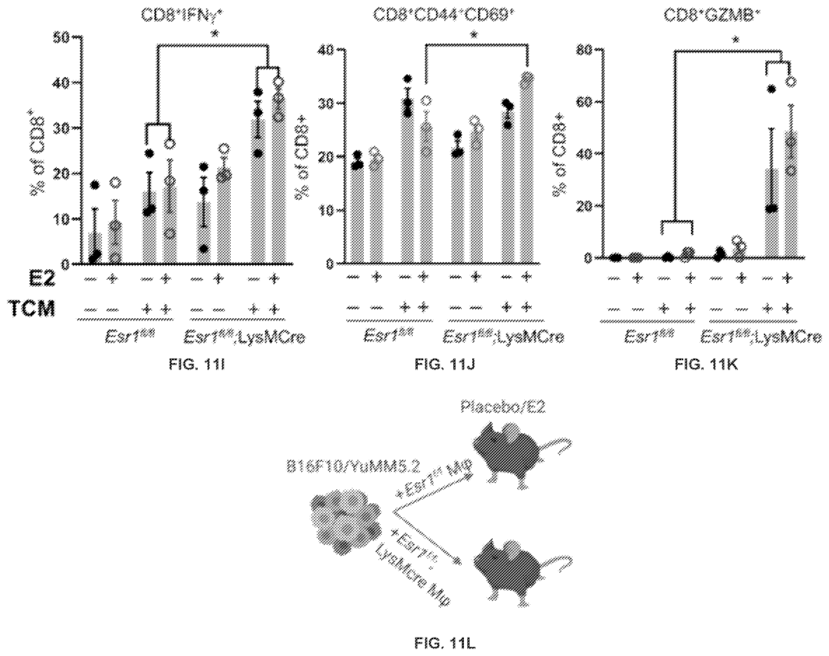

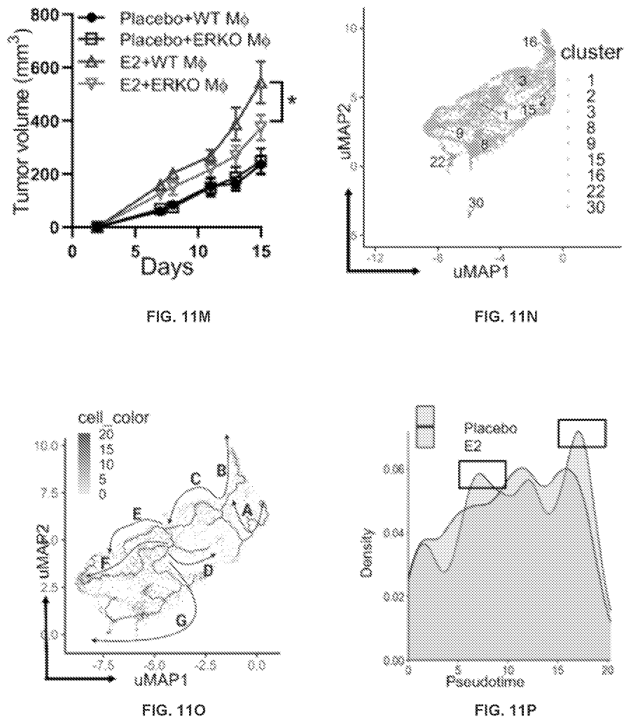

Flow cytometry was used to characterize the myeloid cells within tumors isolated from iBP mice and from mice engrafted with syngeneic tumors (B16F10), treated with either placebo or E2 (FIG. 10A). Quantitatively the infiltration of immune cells (CD45⁺) was similar in the two models and not impacted by treatment (FIGS. 10B-10C). Qualitative assessments, however, revealed that E2 treatment decreases the ratio of intratumoral immunostimulatory M1 (MHCII^{hi} CD206⁻) macrophages to immunosuppressive M2 (MHCII^{lo} CD206^{+/hi}) macrophages (FIGS. 11A-11C). Of note, no changes in the percentage of Ly6C+/Ly6G+ MDSCs in tumors between the two treatment conditions were observed (FIG. 10D). Depletion of macrophages using clodronate liposomes decreased melanoma tumor growth in E2 treated mice but was without any effect in placebo treated mice (FIG. 11D and FIG. 10E). To demonstrate a direct effect of E2 on macrophage polarization (and function), bone marrow progenitor cells were differentiated into macrophages in the presence of M-CSF and either normal media or 30% tumor conditioned media (TCM) from B16F10 cells. The addition of tumor conditioned media allows the TME to be partially mimicked where tumor derived factors influence the differentiation and polarization of macrophages. Following differentiation, macrophages were treated acutely with either DMSO or E2 (1 nM) and then polarized to an M2 state by the addition of IL4. The polarized macrophages were subsequently co-cultured with sub-optimally activated T cells (CD3/CD28 and IL2) isolated from spleens of non-tumor bearing mice, for 72 hr following which they were treated with protein transport inhibitors (monensin and brefeldin) for 6 hr to prevent release of cytokines and chemokines. Flow cytometry analysis revealed that T cells which were co-incubated with either placebo or E2 (1 nM) treated macrophages in normal media (NM) did not display any change in the expression of IFNγ and GZMB. The basal expression of GZMB and IFNγ in T cells was increased significantly upon exposure to macrophages cultured in TCM. However, when T cells were co-incubated with E2 (1 nM) treated macrophages differentiated in TCM, they show a decreased expression of GZMB and IFNγ compared to T cells that were co-incubated with DMSO treated macrophages (FIGS. 11E-11F). These results indicate that E2 treatment induced an immune-suppressive phenotype in tumor conditioned macrophages, which in turn suppressed the cytotoxic capabilities of T cells. However, in the absence of TCM, macrophages did not affect T cell activity even in the presence of E2.

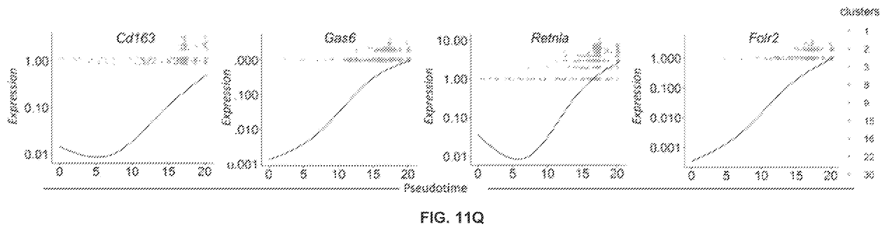

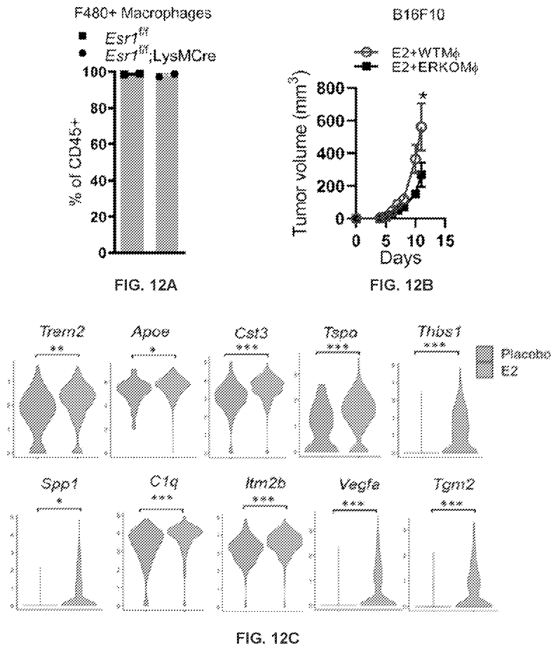

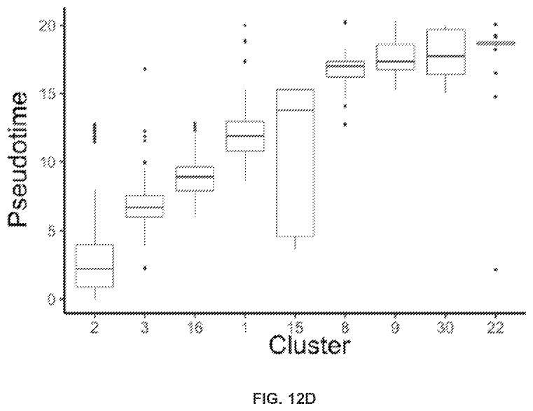

To further explore the roles of ERα in macrophage polarization, bone marrow progenitor cells from Esr1^{f/f} and Esr1^{f/f}; LysMCre animals were isolated and differentiated to bone marrow-derived macrophages (BMDM) in NM or 30% TCM (B16F10). The differentiated BMDM from both Esr1^{f/f} and Esr1^{f/f}; LysMCre genotypes were treated with either DMSO or E2 and then polarized to M2 macrophages by the addition of IL4 (24 hr). These macrophages were then co-incubated for 72 hr with CFSE and sub-optimally activated T cells isolated from non-tumor bearing mouse spleens. Quantification of CFSE dilution demonstrated a significant attenuation of T cell proliferation after incubating with BMDMs compared to T cells alone. No difference in the proliferation of T cells was observed when T cells were co-incubated with macrophages differentiated in NM, regardless of the genotypes of the BMDM and treatments. However, using BMDMs differentiated in TCM, a significant increase in proliferation (CFSE^{low/-}), activation (CD44⁺D69⁺) and cytotoxic (IFNγ⁺ and GZMB⁺) markers was observed when T cells were incubated with BMDM derived from Esr1^{f/f}; LysMCre mice compared to Esr1^{f/f} mice irrespective of the presence or absence of E2 (FIGS. 11G-11K). These results demonstrated that the depletion of ERα in the macrophages enhanced their capacity to promote proliferation of cytotoxic T cells (GZMB⁺ and IFNγ). However, in contrast to previous experiments where a decrease in GZMB and IFNγ expression in T cells was observed upon co-incubation with E2 treated macrophages, T cells did not show similar decrease in the expression of these cytotoxic T cell markers when co-incubated with E2 treated ERα^{f/f} macrophages (FIGS. 11E-11F vs. FIG. 11I and FIG. 11K). It may be due to differences in the underlying genetics (Esr1^{f/f} vs WT). The importance of ERα signaling in macrophages in modulating melanoma tumor growth was further probed in vivo by co-injecting YuMM5.2 or B16F10 tumor cells together with BMDM (FIG. 12A) from either Esr1^{f/f} or Esr1^{f/f}; LysMCre mice (1:1) (FIG. 11L) into syngeneic ovariectomized C57BL/SJ mice treated placebo or E2. The tumor promoting effects of E2 were significantly compromised when tumors (YuMM5.2 and B16F10) were implanted with BMDM from Esr1^{f/f}; LysMCre animals versus Esr1^{f/f} animals (FIG. 11M and FIG. 12B). Taken together, these results indicated the E2/ERα signaling axis in macrophages cooperated with tumor derived factors to promote the establishment of an immune-suppressive TME that facilitated melanoma tumor growth.

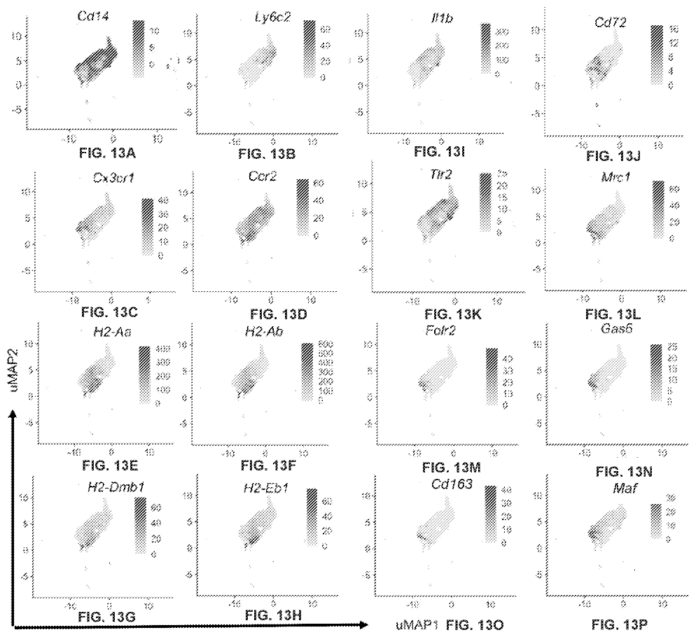

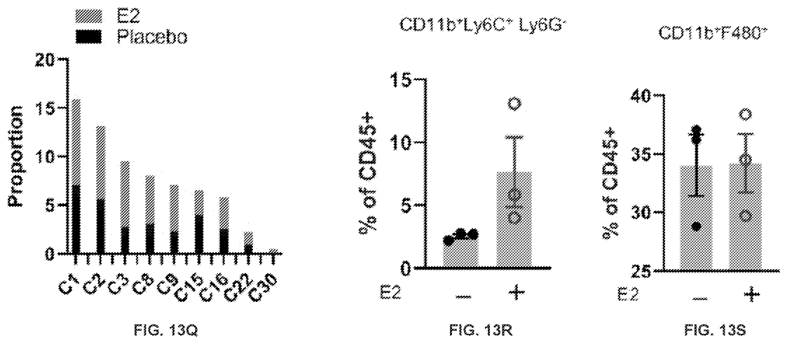

Examination of the scRNA seq profiles revealed that the CD68⁺ monocyte/TAM population from E2 treated tumors expressed markers that were previously reported to be selectively upregulated in TAMs vs. macrophages isolated from the lungs of non-tumor bearing mice (Trem2, Apoe, Thbs1, Spp1) (FIG. 12C). Genes associated with inflammation and those encoding select chemokines (Itm2b, C1q) and M2 macrophages markers (Tspo, Vegfa, Tgm2) were also upregulated in the CD68+ cells from the E2 group (FIG. 12C). The CD68⁺ population was comprised of cells from 9 different clusters (clusters 1, 2, 3, 8, 9, 15, 16, 22, and 30) (FIG. 11N). Analyzing the developmental trajectories of the macrophage/monocyte populations by pseudotime analysis (FIG. 12D) revealed several major branches representing different clusters of cells emerging from monocytes (FIG. 11O). Among these populations, clusters 2, 3, and 16 expressed the monocytic markers Cd14 (FIG. 13A) with cluster 2 (arrow A) showing higher expression of Ly6c2 (FIG. 11O and FIG. 13B). The cluster 2 (arrow A) population then bifurcated into two branches, cluster 3 (arrow C) and cluster 16 (arrow B), both of which expressed intermediate levels of Cx3cr1 (FIG. 13C) but cluster 3 had higher expression of Ccr2 (FIG. 13D) compared to cluster 16. Thus, cluster 3 likely represented inflammatory monocytes while cluster 16 was more similar to patrolling tissue resident monocytes. Of note, both cluster 3 and 16 were increased in E2 treated tumors compared to placebo treatment (pseudotime block 5-10, boxed region) while the percentage of Ly6C^{hi} monocytes (cluster 2) remained the same between the two treatments (FIG. 11P and FIG. 13B). Cluster 3 further proceeded to a major branching point leading to the formation of 4 different trajectories, mainly cluster 15 (arrow D), cluster 1 (arrow E), cluster 9 (arrow F), and 8, 22, and 30 (arrow G) (FIG. 11O). Among these clusters, 1, 8, 22, 30, and 15 all expressed genes associated with the MHCII complex (H2-Aa, H2-Ab, H2-Dmb1, and H2-Eb) (FIGS. 13E-13H). Cluster 1 and 15 additionally expressed inflammatory genes Il1b (FIG. 131) and likely comprised of inflammatory or “M1-like” TAMs. While cluster 1 remained unchanged, cluster 15 decreased upon E2 treatment (FIG. 13Q). Clusters 8, 22, and 30 expressed inflammatory genes (Cd72 and Ir2) (FIG. 12J-12K) in addition to genes of MHCII complex, however they also expressed genes associated with M2 macrophages (Mrc1) (FIG. 12L). While the exact functionality of these macrophage subsets was not clear, phenotypically they are analogous to the population of circulating cells of monocyte/macrophage lineage that express markers of both M1 and M2 cell phenotypes as reported previously. Within these clusters, cluster 8 and cluster 30 showed expansion upon E2 treatment, while cluster 22 remained unchanged (FIG. 13Q). Cluster 9 was a notable exception, which expressed markers associated with immune-suppressive phenotype (Mrc1, Folr2, Gas6, RetnMa, and Cd163) (FIG. 11Q and FIGS. 13M-13O). This cluster also showed higher expression of Maf, a gene which is required for differentiation of monocytes to macrophages (FIG. 13P). Importantly, cluster 9 showed significant expansion with E2 treatment compared to placebo (FIG. 13Q). This observation supported the hypothesis that E2 treatment leads to the expansion of macrophages that demonstrate immune-suppressive phenotypes. Taken together, this analysis suggested that E2 may promote the initial recruitment of monocytes, as evidenced by increase in cluster 3 to the tumor microenvironment where the monocytes exposed to tumor derived factors and E2 undergo faster rates of differentiation and polarization to M2 macrophages (cluster 9) while at the same time suppresses expansion of M1 macrophages (cluster 15). This result was further supported by the flow cytometry data where a trend was observed towards an increase in the number of monocytes in response to E2 (FIG. 13R) and a decrease in M1/M2 ratio with the total number of F480⁺ macrophages remaining unchanged (FIGS. 11A-11B and FIG. 13S).

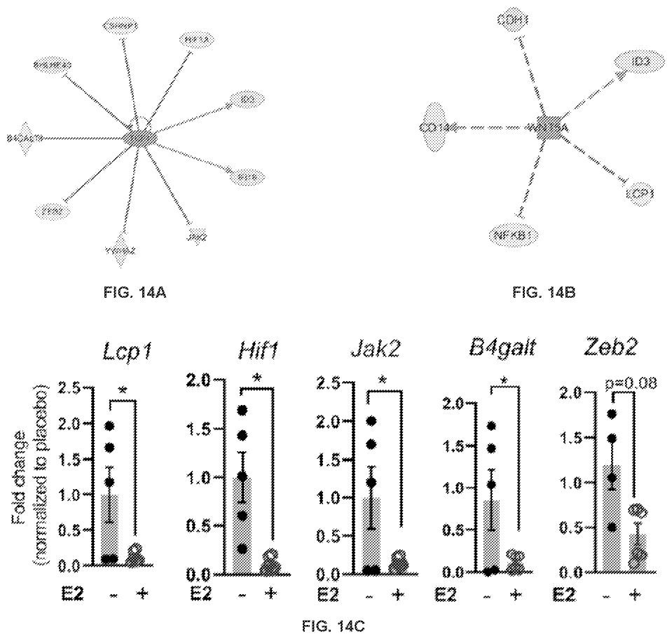

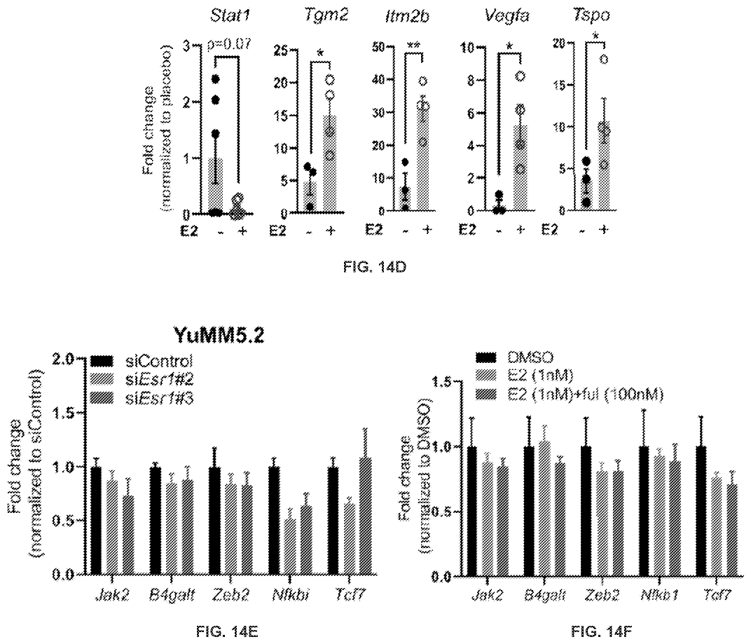

To determine the molecular pathway(s) that influence this M2 phenotype in E2 treated macrophages, upstream regulator analysis was performed of differentially expressed genes (DEGs) in CD68+ cells using Ingenuity Pathway Analysis (IPA). This analysis highlighted the importance of the TCF4 and WNTSA pathways (FIGS. 14A-14B), the significance of which was explored in tumor infiltrating myeloid cells isolated from iBP tumors excised from mice treated with placebo or E2. Gene expression analysis revealed that multiple genes in the WNT5A and TCF4 pathways were differentially regulated by E2 compared to placebo in these cells (FIG. 14C). WNTSA, signaling through the canonical β-catenin pathway, has been implicated in various biological processes including embryogenesis, cell fate development, and endothelial cell differentiation resulting in the upregulation of vasculogenic and angiogenic processes, although the significance of E2 in the regulation of these processes in the TME remains to be determined. Of note, WNT5A signaling has also been reported to induce tolerogenic phenotypes in macrophages in breast cancer patients. Myeloid cells isolated from E2 treated tumors were demonstrated to manifest a gene expression pattern characteristic of M2 macrophages with increased expression of multiple genes, such as Vegfa, Tgm2 and Tspo and Stat1 (FIG. 14D). It has yet to be determined whether E2-regulated expression of these genes depends on WNT signaling. In contrast to myeloid cells, knockdown of Esr1 or treatment with either E2 (1 nM) or E2 (1 nM)+fulvestmat (100 nM) did not change the expression of WNTSA-β-catenin targets in YuMM5.2 cells (FIGS. 14E-14F), although EVER signaling has previously been shown to influence 1-catenin signaling in cancer cells. Together, these results indicated a likely role for E2 in the functional activation of WVNT5A-β-catenin signaling leading to macrophage polarization towards an immune-suppressive state in the melanoma tumor microenvironment.

### Example 6

### E2 Treatment Suppresses Anti-Tumor T Cell Responses

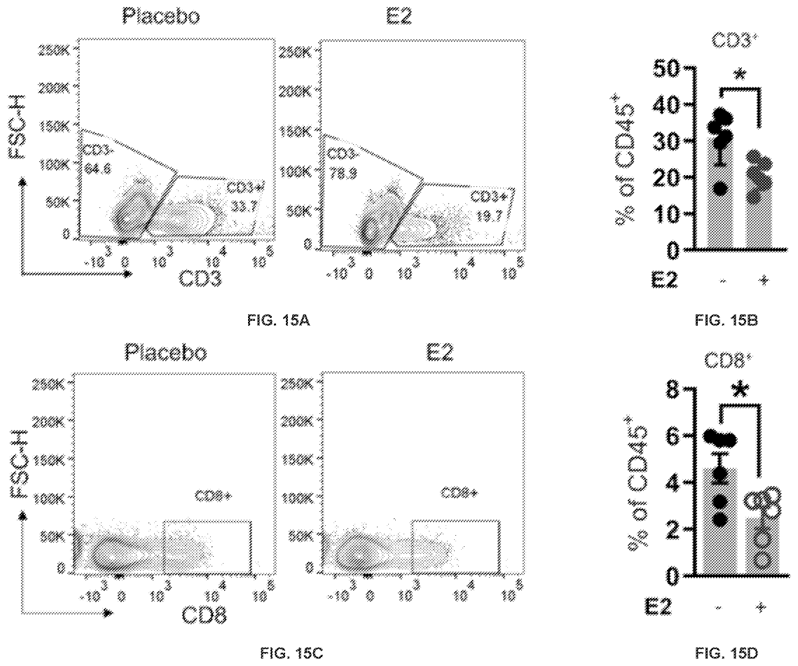

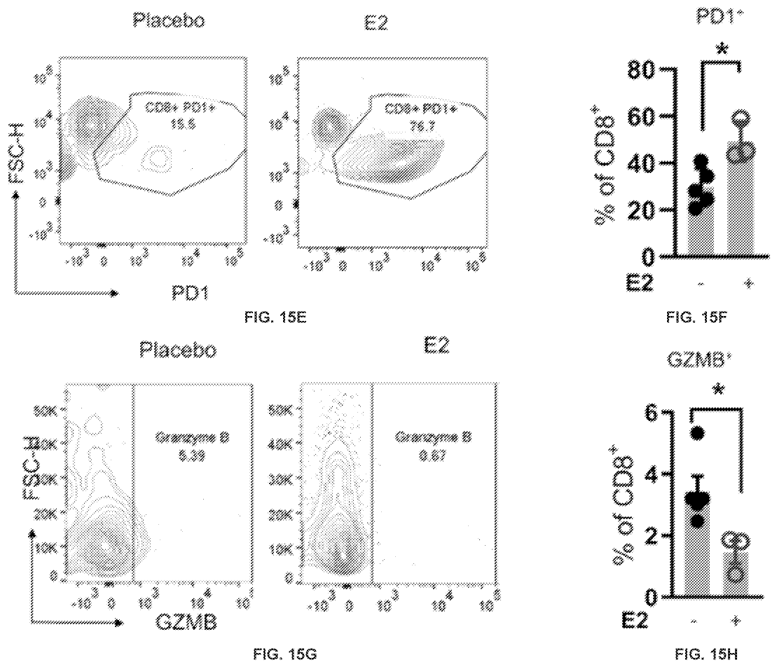

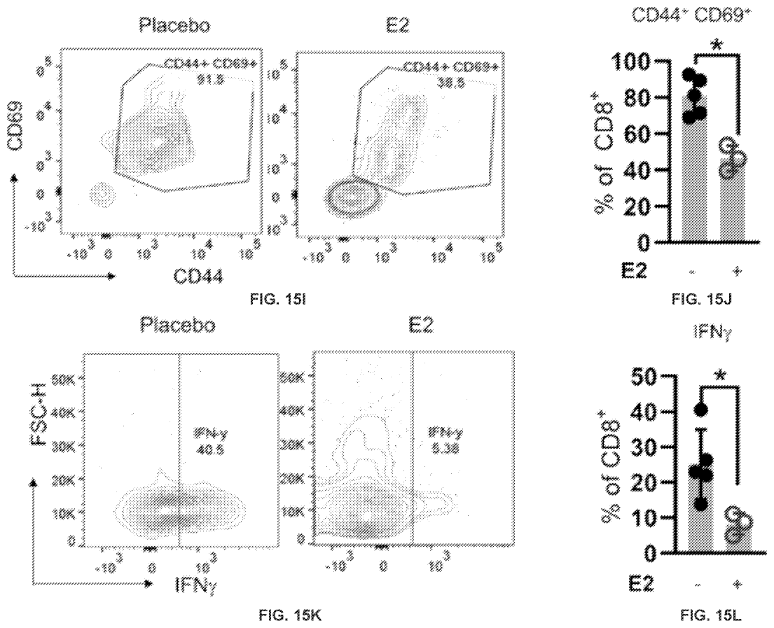

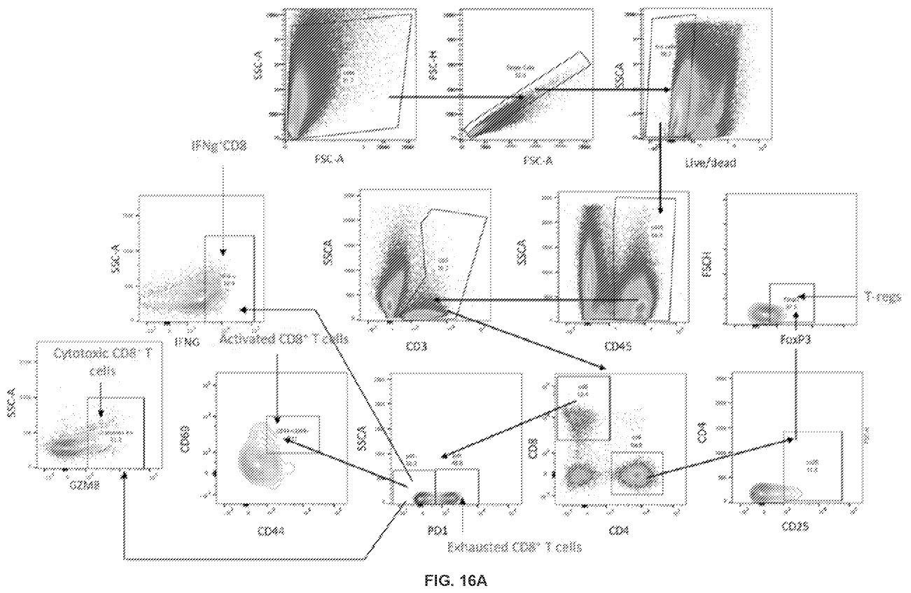

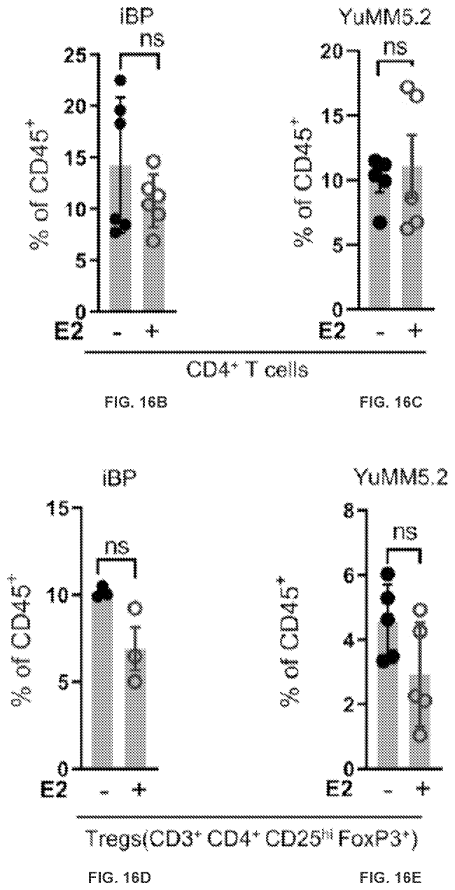

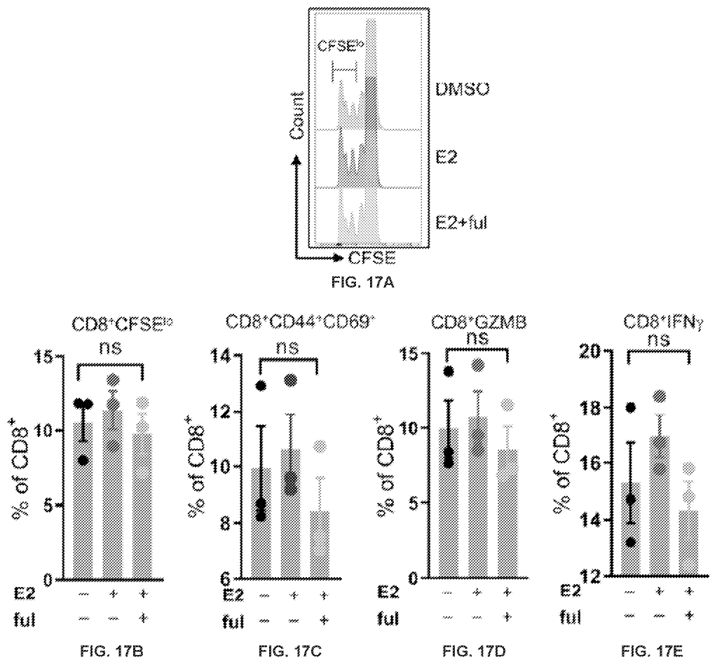

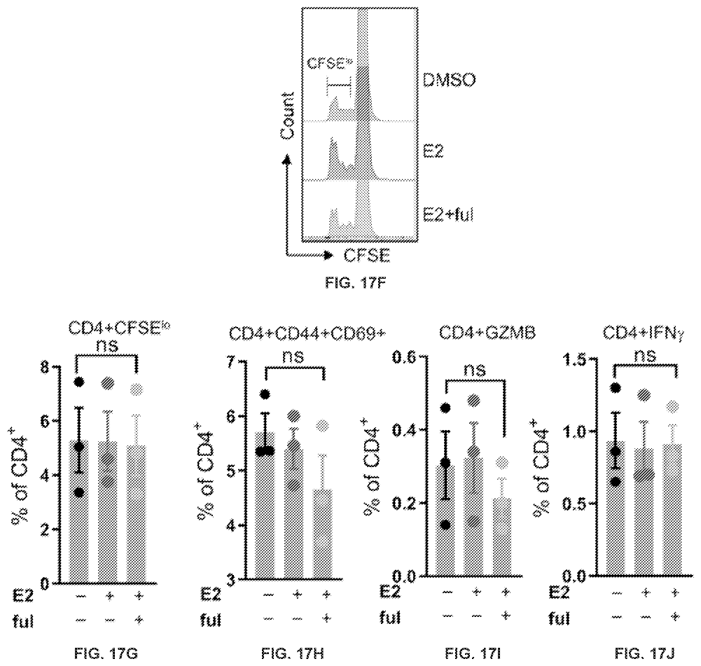

The results of the ex vivo studies described above suggested that E2 exerts a direct effect on macrophages to suppress the proliferation and activity of both CD4⁺ and CD8⁺ T cells. Flow cytometry analysis of tumor-infiltrating T cells from iBP tumors also revealed an overall decrease in the CD3⁺ T cell population with E2 treatment (FIGS. 15A-15B and FIG. 16A). Further, sub-gating of the CD3⁺ positive T cell population indicated that the number of intra-tumoral CD8⁺ cytotoxic T cells were decreased upon E2 treatment, while no significant changes in CD4⁺ T cells were observed (FIGS. 15C-15D and FIGS. 16B-16C). The activity of tumor infiltrating T cells was also evaluated using CD3⁺ T cells isolated from syngeneic YuMM5.2 tumors. For this purpose, T cells were isolated from placebo and E2 treated tumors and ex vivo treated with PMA and ionomycin for 4 hr along with protein transport inhibitors. Flow cytometry analysis demonstrated that when compared to T cells isolated from placebo treated mice, the CD8⁺ tumor infiltrating lymphocytes (TILs) isolated from E2 treated YuMM5.2 tumors were markedly more exhausted, expressing significantly more PD1 (FIGS. 15E-15F) and reduced expression of Granzyme B, (FIGS. 15G-15H), activation markers CD44 and CD69 (FIGS. 151-15J), and cytokines such as IFNγ (FIGS. 15K-15L). As in the iBP model, a significant impact of E2 treatment on the infiltration of CD4⁺ FOXP3⁺ regulatory T cell subsets was not observed (FIGS. 16D-16E). When taken together, these results suggested that systemic E2 treatment reduces T cell functionality albeit in an indirect manner as Esr1, Esr2 or Gper1 RNA were not expressed in T cells within the tumor microenvironment (FIGS. 8B-8D). Further, treatment of T cells in vitro with either E2 or the SERD fulvestrant did not affect the proliferation or cytotoxic capabilities of either CD4⁺ or CD8⁺ T cells (FIGS. 17A-17J). Taken together, these data indicated that E2 indirectly reduced T cell function secondary to its effects on macrophages.

### Example 7

### Pharmacological Inhibition of ER Reverses the Growth Promoting Effects of E2 on Melanoma Tumors

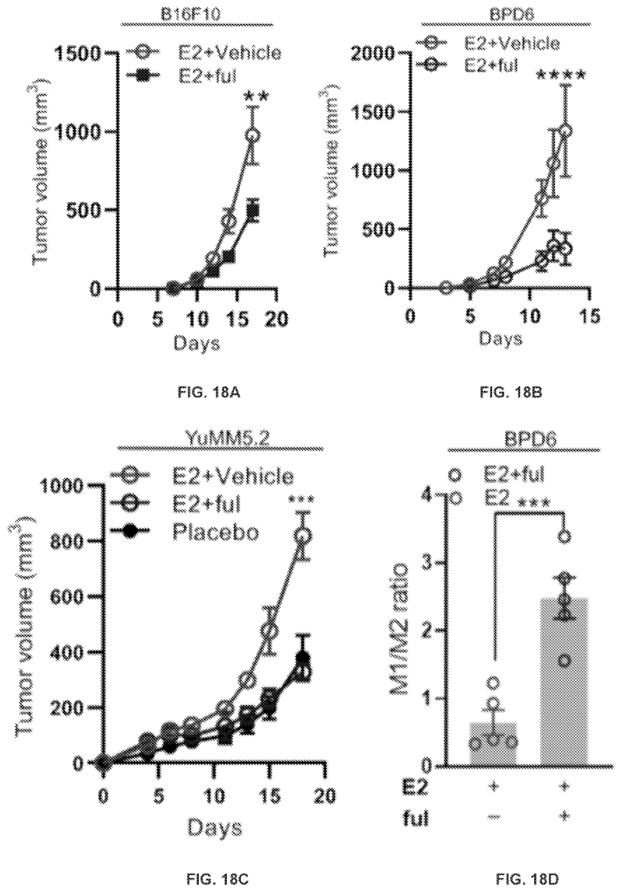

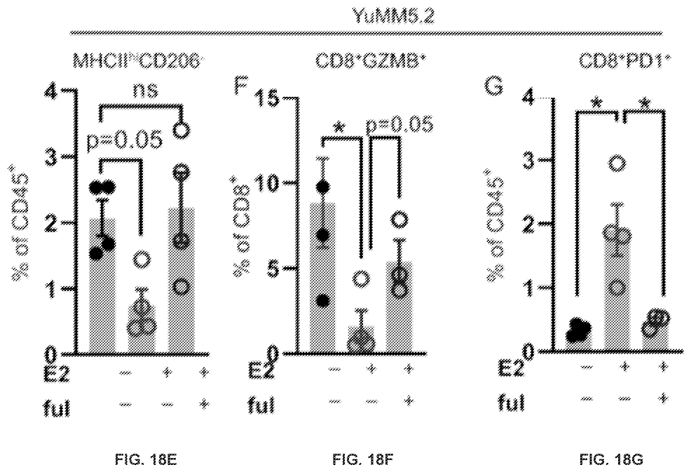

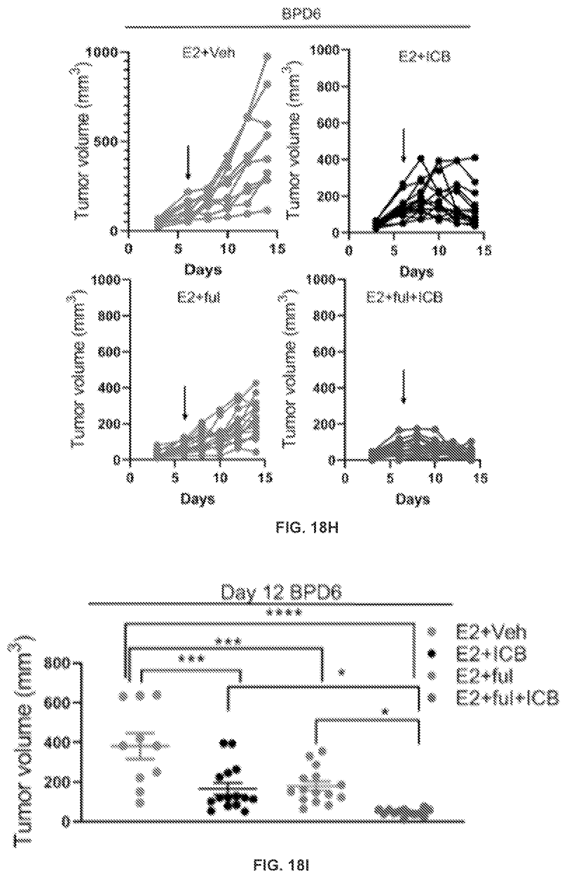

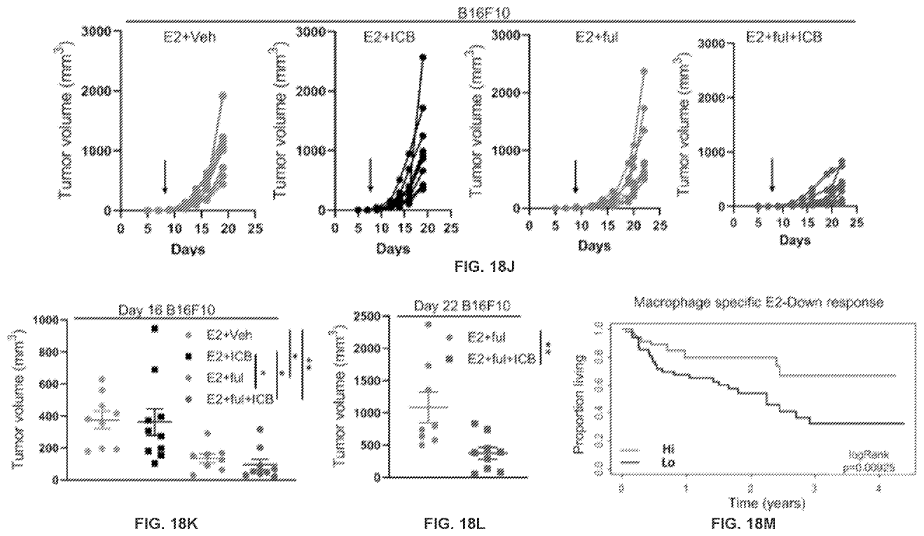

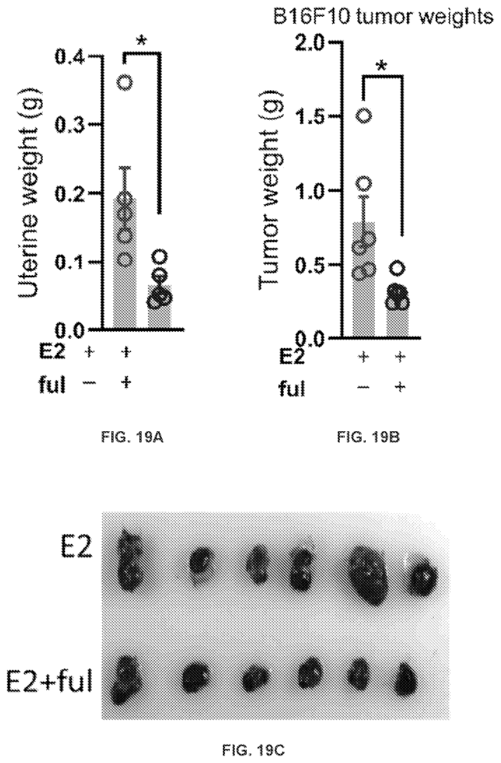

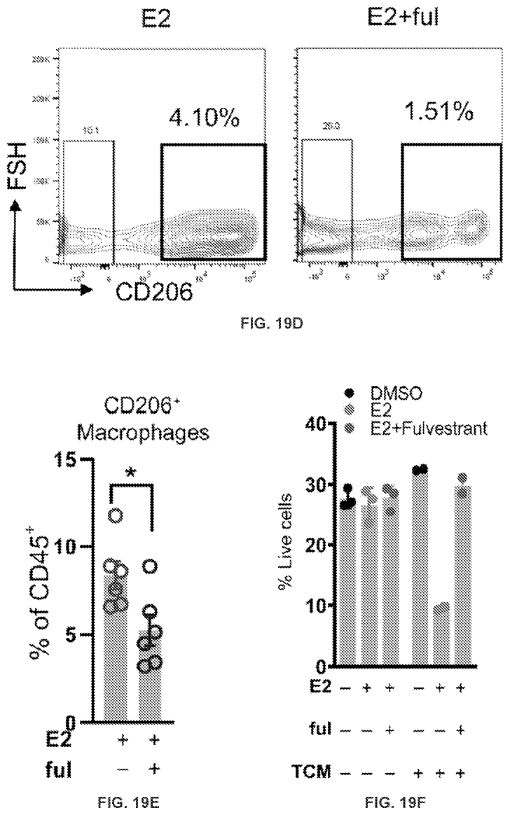

Fulvestrant, a SERD, acts by both inactivating and degrading ER and is approved for use in post-menopausal patients with ER-positive breast cancer who have progressed on first-line endocrine therapies. It was selected for these studies as it is the most efficacious ER inhibitor currently available for clinical use. At a dose that was determined to model achievable levels in breast cancer patients (25 mg/kg), fulvestrant significantly reduced tumor growth in all preclinical models of melanoma examined (B16F10, YuMM5.2, and BPD6) (FIGS. 18A-18C and FIGS. 19A-19C). To understand how fulvestrant affects the TME, the tumor infiltrating immune cell repertoire was analyzed by flow cytometry. An increase in intratumoral M1/M2 ratio or an increase in inflammatory macrophages (MHCII^{hi} CD206⁻) was observed when E2 treated mice were co-treated with fulvestrant (FIGS. 18D-ISE and FIGS. 19D-19E). Tumor infiltrating T cells from fulvestrant treated tumors displayed an increase in cytotoxic capabilities as measured by Granzyme B (GZMB) expression (FIG. 18F). Additionally, fulvestrant treatment led to a decrease in the number of PD1⁺CD8⁺ T cells (exhausted T cells) that increased with E2 treatment (FIG. 18G). Similar observations were made in studies performed in vitro when BMDM cells treated with fulvestrant were co-incubated with CFSE-labelled sub-optimally activated (CD3/CD28) T cells in presence of IL2. Analysis of CFSE dilution revealed that the proliferation of T cells was not affected by their co-incubation with macrophages differentiated in NM and treated with either E2 or E2+fulvestrant. However, T cells exposed to macrophages, differentiated in 30% TCM and E2, effectively suppressed T cell proliferation, an activity that was reversed by treatment with fulvestrant (FIG. 19F). Collectively, these results indicated that fulvestrant can inhibit the effects of E2 on tumor growth and remodel the tumor immune microenvironment to favor tumor growth inhibition in melanoma.

Studies were then conducted to evaluate whether fulvestrant improves/restores response to the immune checkpoint inhibitor, α-PD1, in the PD1 sensitive BPD6 and unresponsive B16F10 tumor model. In the PD1 sensitive BPD6 model, treatment with either fulvestrant or ICB (α-PD1 and α-CTLA4) slows tumor growth, however the combination of both drugs further suppressed tumor growth when compared to each individual treatment (FIGS. 18H-18I). To determine whether fulvestrant can also increase the effectiveness of immunotherapy in ICB unresponsive B16F10 model, mice with established B16F10 tumors were treated with fulvestrant and α-PD1 either alone or in combination. Importantly, the combination of fulvestrant with α-PD1 suppressed the growth of B16F10 tumors, while PD1 treatment alone was without any effect (FIGS. 18J-18L). Taken together these results indicate that pharmacological targeting of ERα can improve the intratumoral M1/M2 ratio and increase the effectiveness of ICB in both ICB sensitive and resistant models of melanoma. Since E2-driven tumor growth appears to be macrophage dependent, it was anticipated that a macrophage specific ERα signature would predict ICB sensitivity in melanoma patients. To this end, the E2-regulated genes in all CD68⁺ macrophage/monocyte clusters identified from scRNA seq were first divided into 2 groups: genes upregulated by E2 (E2-Up response) and genes down regulated by E2 (E2-Down response) (TABLE 5). The human orthologs of the identified murine signatures were then used to predict survival of patients receiving ICB treatments using publicly available transcriptional datasets from patients receiving ICB treatments. It was observed that an enrichment of macrophage specific-E2 down regulated genes (E2-Down) correlated with a better overall survival in melanoma patients who have received ICB (FIG. 18M). These results highlighted the importance of ERα function in TAMs residing in melanoma TME and demonstrated how an ERα specific signature can be utilized to predict a patient's response to ICB treatments.

TABLE 5
E2 up- and down-regulated DEGs in CD68+ cells.

| | | | | | |
|---|---|---|---|---|---|
| Gene | p_val | avg_logFC | pct. 1 | pct. 2 | p_val_adj |

Genes down-regulated upon E2 treatment

| | | | | | |
|---|---|---|---|---|---|
| Gm42418 | 4.01E−99 | −0.30987 | 1 | 1 | 1.92E−94 |
| Pabpc1 | 3.29E−61 | −0.30645 | 0.986 | 0.977 | 1.57E−56 |
| Ifi202b | 9.77E−56 | −0.45888 | 0.255 | 0.511 | 4.66E−51 |
| Rpl23a | 2.64E−52 | −0.33947 | 0.912 | 0.936 | 1.26E−47 |
| Clec7a | 4.90E−48 | −0.68226 | 0.765 | 0.877 | 2.34E−43 |
| Prkcd | 8.90E−46 | −0.4952 | 0.865 | 0.921 | 4.25E−41 |
| Qsox1 | 7.86E−43 | −0.29814 | 0.152 | 0.348 | 3.75E−38 |
| Ywhaz | 2.14E−40 | −0.34652 | 0.881 | 0.923 | 1.02E−35 |
| Mir6236 | 8.77E−38 | −0.47949 | 0.909 | 0.932 | 4.18E−33 |
| Arg1 | 6.62E−30 | −1.49079 | 0.121 | 0.268 | 3.16E−25 |
| Gm13339 | 2.23E−28 | −0.32348 | 0.692 | 0.769 | 1.07E−23 |
| Hnrnpa2b1 | 6.74E−27 | −0.25075 | 0.936 | 0.934 | 3.22E−22 |
| Ddx3x | 1.10E−26 | −0.26479 | 0.832 | 0.882 | 5.27E−22 |
| Lcp1 | 1.56E−26 | −0.27897 | 0.961 | 0.965 | 7.43E−22 |
| Cd44 | 2.53E−25 | −0.36365 | 0.825 | 0.883 | 1.21E−20 |
| Dazap2 | 6.58E−25 | −0.25305 | 0.897 | 0.933 | 3.14E−20 |
| Rnf19b | 1.65E−24 | −0.40714 | 0.455 | 0.593 | 7.88E−20 |
| Arl4c | 2.26E−24 | −0.32545 | 0.817 | 0.853 | 1.08E−19 |
| Rbms1 | 4.29E−24 | −0.30603 | 0.862 | 0.881 | 2.05E−19 |
| Bhlhe40 | 1.05E−23 | −0.42137 | 0.388 | 0.543 | 5.00E−19 |
| Cbl | 1.81E−23 | −0.25872 | 0.753 | 0.819 | 8.63E−19 |
| Actg1 | 3.24E−23 | −0.27253 | 0.997 | 0.991 | 1.55E−18 |
| Lars2 | 3.72E−23 | −0.27375 | 0.816 | 0.854 | 1.77E−18 |
| Hk2 | 2.20E−22 | −0.31606 | 0.59 | 0.7 | 1.05E−17 |
| Cdh1 | 2.81E−22 | −0.46823 | 0.065 | 0.17 | 1.34E−17 |
| Ass1 | 2.88E−21 | −0.29806 | 0.125 | 0.251 | 1.38E−16 |
| Cd53 | 9.47E−21 | −0.28312 | 0.925 | 0.93 | 4.52E−16 |
| Hnrnpu | 9.81E−21 | −0.2502 | 0.656 | 0.745 | 4.68E−16 |
| Zeb2 | 4.16E−19 | −0.27039 | 0.953 | 0.949 | 1.98E−14 |
| Malt1 | 4.79E−19 | −0.44968 | 0.255 | 0.383 | 2.28E−14 |
| Rbbp6 | 8.16E−19 | −0.26927 | 0.585 | 0.68 | 3.90E−14 |
| Tes | 2.93E−18 | −0.26628 | 0.253 | 0.379 | 1.40E−13 |
| AA467197 | 9.88E−18 | −1.1474 | 0.121 | 0.23 | 4.72E−13 |
| Hexim1 | 1.18E−16 | −0.25983 | 0.511 | 0.621 | 5.63E−12 |
| Adam8 | 1.26E−16 | −0.53147 | 0.345 | 0.462 | 6.02E−12 |
| Plek | 2.00E−16 | −0.31211 | 0.837 | 0.87 | 9.57E−12 |
| Jak2 | 8.82E−16 | −0.32942 | 0.364 | 0.474 | 4.21E−11 |
| Zfp36l2 | 1.44E−14 | −0.27323 | 0.941 | 0.9 | 6.87E−10 |
| Ccl24 | 3.00E−14 | −1.19584 | 0.13 | 0.227 | 1.43E−09 |
| Slc7a11 | 3.97E−14 | −0.38456 | 0.161 | 0.265 | 1.90E−09 |
| Tapbp | 2.06E−13 | −0.25423 | 0.786 | 0.829 | 9.83E−09 |
| Epb41l2 | 2.70E−13 | −0.26678 | 0.426 | 0.525 | 1.29E−08 |
| Rbpj | 3.18E−13 | −0.2858 | 0.733 | 0.782 | 1.52E−08 |
| Havcr2 | 3.54E−13 | −0.27376 | 0.287 | 0.388 | 1.69E−08 |
| B4galt1 | 6.35E−13 | −0.25928 | 0.474 | 0.566 | 3.03E−08 |
| Nfkb1 | 9.03E−13 | −0.31868 | 0.72 | 0.769 | 4.31E−08 |
| Cish | 1.09E−12 | −0.28776 | 0.157 | 0.255 | 5.21E−08 |
| Hif1a | 1.10E−12 | −0.30163 | 0.688 | 0.725 | 5.24E−08 |
| N4bp1 | 1.71E−12 | −0.27229 | 0.514 | 0.6 | 8.15E−08 |
| Cox17 | 7.12E−12 | −0.25158 | 0.802 | 0.814 | 3.40E−07 |
| Ero1l | 8.97E−12 | −0.36815 | 0.267 | 0.36 | 4.28E−07 |
| Tgfbi | 9.25E−12 | −0.28736 | 0.959 | 0.941 | 4.42E−07 |
| Etv3 | 1.07E−11 | −0.25332 | 0.439 | 0.54 | 5.11E−07 |
| Mmp14 | 3.05E−11 | −0.42337 | 0.292 | 0.385 | 1.46E−06 |
| Rgcc | 4.49E−11 | −0.33561 | 0.197 | 0.295 | 2.14E−06 |
| Slc7a2 | 1.60E−10 | −0.45817 | 0.073 | 0.141 | 7.64E−06 |
| Hbegf | 1.88E−10 | −0.28825 | 0.052 | 0.111 | 8.96E−06 |
| Rhob | 9.86E−10 | −0.34427 | 0.416 | 0.499 | 4.71E−05 |
| Dusp5 | 1.08E−09 | −0.26439 | 0.391 | 0.478 | 5.17E−05 |
| Nampt | 1.54E−09 | −0.25391 | 0.404 | 0.478 | 7.35E−05 |
| Csrnp1 | 1.87E−09 | −0.27956 | 0.541 | 0.609 | 8.91E−05 |
| Slc2a1 | 3.02E−09 | −0.27258 | 0.302 | 0.377 | 0.000144 |
| Mafb | 4.04E−09 | −0.27317 | 0.811 | 0.842 | 0.000193 |
| Il1rn | 1.92E−08 | −0.70865 | 0.284 | 0.356 | 0.000918 |
| Itgax | 6.41E−08 | −0.27182 | 0.229 | 0.308 | 0.003058 |
| Scd2 | 1.30E−07 | −0.32672 | 0.309 | 0.377 | 0.006206 |
| Smox | 7.56E−07 | −0.27427 | 0.307 | 0.373 | 0.036086 |
| Fam129b | 1.02E−06 | −0.27316 | 0.488 | 0.537 | 0.048555 |
| Clic4 | 1.16E−06 | −0.28359 | 0.512 | 0.563 | 0.055551 |
| Hilpda | 3.80E−06 | −0.38177 | 0.159 | 0.218 | 0.181233 |
| Bnip3 | 4.87E−06 | −0.32411 | 0.224 | 0.28 | 0.232537 |
| Gatm | 9.90E−06 | −0.47277 | 0.389 | 0.436 | 0.472454 |
| Retnla | 0.000216 | −1.01252 | 0.114 | 0.157 | 1 |
| Sdc4 | 0.000116 | −0.57293 | 0.422 | 0.442 | 1 |
| Spp1 | 0.012489 | −0.53669 | 0.241 | 0.2 | 1 |
| Timp1 | 0.109847 | −0.39221 | 0.102 | 0.118 | 1 |
| Srgn | 0.003933 | −0.37507 | 0.956 | 0.938 | 1 |
| Slpi | 0.760551 | −0.36953 | 0.251 | 0.248 | 1 |
| Tgm2 | 0.003778 | −0.34501 | 0.525 | 0.539 | 1 |
| Id2 | 0.000906 | −0.32347 | 0.497 | 0.526 | 1 |
| Lyz1 | 0.802287 | −0.31085 | 0.11 | 0.109 | 1 |
| Cxcl2 | 0.080087 | −0.31077 | 0.436 | 0.396 | 1 |
| Emp1 | 0.007151 | −0.30161 | 0.245 | 0.278 | 1 |
| Ighm | 0.005633 | −0.26937 | 0.449 | 0.478 | 1 |
| Errfi1 | 0.007164 | −0.26125 | 0.333 | 0.362 | 1 |
| Ptgs2 | 0.418583 | −0.2589 | 0.281 | 0.294 | 1 |
| Grina | 0.040204 | −0.25269 | 0.609 | 0.597 | 1 |

Genes up-regulated upon E2 treatment

| | | | | | |
|---|---|---|---|---|---|
| Fcer1g | 1.70E−73 | 0.313666 | 0.991 | 0.959 | 8.12E−69 |
| Rps20 | 3.20E−71 | 0.292162 | 0.988 | 0.963 | 1.53E−66 |
| Rplp0 | 3.21E−70 | 0.333023 | 0.988 | 0.963 | 1.53E−65 |
| Gm10076 | 3.18E−66 | 0.375729 | 0.776 | 0.536 | 1.52E−61 |
| Cst3 | 2.22E−63 | 0.344584 | 0.993 | 0.974 | 1.06E−58 |
| Rpl9 | 2.26E−55 | 0.258515 | 0.99 | 0.967 | 1.08E−50 |
| Rps11 | 9.76E−55 | 0.270445 | 0.992 | 0.967 | 4.66E−50 |
| Itm2b | 2.29E−51 | 0.410393 | 0.988 | 0.955 | 1.09E−46 |
| Rps2 | 2.13E−46 | 0.274231 | 0.982 | 0.947 | 1.02E−41 |
| Rpl3 | 7.06E−45 | 0.277989 | 0.983 | 0.947 | 3.37E−40 |
| Rpl29 | 1.93E−44 | 0.254772 | 0.981 | 0.939 | 9.21E−40 |
| Rpl21 | 4.96E−43 | 0.256687 | 0.975 | 0.93 | 2.37E−38 |
| Hint1 | 9.37E−43 | 0.254822 | 0.949 | 0.886 | 4.47E−38 |
| H2-D1 | 1.74E−42 | 0.280774 | 0.993 | 0.978 | 8.30E−38 |
| Ifitrn2 | 7.32E−42 | 0.373988 | 0.963 | 0.892 | 3.50E−37 |
| Dpep2 | 7.61E−41 | 0.372547 | 0.635 | 0.403 | 3.63E−36 |
| Chil3 | 1.08E−36 | 1.252599 | 0.497 | 0.324 | 5.15E−32 |
| Thbs1 | 1.21E−36 | 0.31892 | 0.754 | 0.55 | 5.79E−32 |
| Trem2 | 2.05E−36 | 0.483108 | 0.736 | 0.547 | 9.79E−32 |
| Cd48 | 8.60E−33 | 0.271492 | 0.77 | 0.586 | 4.11E−28 |
| Gm10222 | 1.08E−31 | 0.257707 | 0.924 | 0.838 | 5.14E−27 |
| Apoe | 5.96E−29 | 0.56897 | 0.91 | 0.834 | 2.85E−24 |
| Ifi27l2a | 5.41E−28 | 0.524508 | 0.832 | 0.734 | 2.58E−23 |
| Tspo | 2.97E−26 | 0.26039 | 0.949 | 0.888 | 1.42E−21 |
| Al607873 | 3.82E−26 | 0.278624 | 0.669 | 0.487 | 1.82E−21 |
| Ckb | 6.26E−26 | 0.284662 | 0.544 | 0.351 | 2.99E−21 |
| Fcrls | 1.22E−25 | 0.563825 | 0.363 | 0.181 | 5.82E−21 |
| Tmem176 | 1.42E−24 | 0.320935 | 0.827 | 0.711 | 6.76E−20 |
| Bst2 | 4.38E−23 | 0.296136 | 0.727 | 0.565 | 2.09E−18 |
| Itga6 | 6.45E−23 | 0.343685 | 0.427 | 0.257 | 3.08E−18 |
| Lyz2 | 2.71E−22 | 0.288498 | 0.987 | 0.972 | 1.30E−17 |
| Tmem176 | 6.13E−22 | 0.339366 | 0.755 | 0.63 | 2.93E−17 |
| AF251705 | 9.56E−22 | 0.263936 | 0.862 | 0.744 | 4.56E−17 |
| Mt1 | 2.24E−21 | 0.400572 | 0.67 | 0.527 | 1.07E−16 |
| Gdf3 | 2.69E−21 | 0.285113 | 0.193 | 0.064 | 1.28E−16 |
| Lrg1 | 3.40E−21 | 0.366331 | 0.15 | 0.036 | 1.62E−16 |
| Rgs10 | 3.29E−20 | 0.259922 | 0.766 | 0.656 | 1.57E−15 |
| Mgl2 | 7.17E−20 | 0.57124 | 0.391 | 0.227 | 3.42E−15 |
| Cfh | 1.63E−16 | 0.262516 | 0.393 | 0.245 | 7.79E−12 |
| Arl5c | 1.13E−12 | 0.265557 | 0.482 | 0.368 | 5.38E−08 |
| Cd14 | 3.49E−12 | 0.271502 | 0.86 | 0.788 | 1.66E−07 |
| Pf4 | 3.97E−12 | 0.471453 | 0.32 | 0.204 | 1.89E−07 |
| C1qb | 4.94E−12 | 0.518768 | 0.516 | 0.428 | 2.36E−07 |
| Ifitrn3 | 2.23E−10 | 0.27548 | 0.91 | 0.856 | 1.07E−05 |
| Ccrl2 | 5.20E−10 | 0.32632 | 0.383 | 0.284 | 2.48E−05 |
| C1qa | 4.09E−09 | 0.42138 | 0.468 | 0.372 | 0.000195 |
| C1qc | 8.10E−09 | 0.420775 | 0.484 | 0.397 | 0.000387 |
| Id3 | 1.63E−08 | 0.269949 | 0.281 | 0.191 | 0.00078 |
| Ly6c2 | 0.312581 | 0.265697 | 0.364 | 0.377 | 1 |
| S100a8 | 9.16E−05 | 0.785754 | 0.138 | 0.088 | 1 |

### Example 8

### Discussion

A tumor cell's extrinsic activity of ERα has been identified that results in an increased accumulation of M2 or alternatively activated macrophages in the TME that suppresses adaptive immunity and promotes tumor growth in murine models of melanoma. Previously, it has been demonstrated that E2 promotes MDSC mobilization to tumor sites and creates an immune-suppressive tumor microenvironment in ovarian, lung, and breast cancer. While there was anecdotal evidence suggesting that elevated numbers of circulating monocytic MDSCs track with Ipilimumab treatment outcome in melanoma patients, the data detailed herein revealed that it is the intratumoral M1/M2 macrophage ratio, and not changes in granulocytic MDSCs, that predicts responses in patients treated with either PD1 or CTLA4 alone or in combination. This encouraged the investigation of the mechanisms by which E2 modulates response to ICBs. Here, evidence is provided demonstrating that removal of endogenous estrogens (ovariectomy) provides a protective advantage against tumor growth in part by decreasing the number of immune suppressive TAMs and by preventing the exhaustion of cytotoxic T cells. This function was primarily attributed to E2/ER signaling in macrophages and their ability to facilitate M2 polarization. Of clinical importance is the finding that the SERD, fulvestrant, can reverse the effects of E2 on tumor growth and immune cell repertoire, establishing the importance of ER in melanoma biology and highlighting a potential new treatment modality for this disease.

Tumor associated macrophages are one of the dominant immune cell types within the TME and can promote tumor growth by increasing neo-vascularization, promoting wound healing/tissue repair processes, and blocking the activation of adaptive immune cells within the TME. TAM recruitment in tumors is generally associated with resistance to chemotherapy and immunotherapy, and thus there is a high level of interest in developing interventional approaches to suppress the immune-suppressive and pro-tumoral activities of these cells. Among the strategies employed and/or under investigation are depletion of TAMs in the TME using CSF1R antibodies or bisphosphonates, prevention of TAM recruitment to tumors by inhibiting the CCL2/CCR2 axis, or reprogramming of TAMs using anti-CD47-SIRPα antibodies, TLR agonists, and inhibitors of the enzyme calcium calmodulin kinase kinase-2. While somewhat successful in different tumor contexts, these therapies have often suffered from severe toxicities that have limited their use in patients. This highlights the potential clinical importance of the observation that estrogens (E2) can promote the establishment and maintenance of a tumor suppressive microenvironment by TAM polarization—an activity that can be reversed by ER antagonist/SERD, fulvestrant.

Estrogens have been shown to play a major role in reducing inflammation by promoting the polarization of macrophages towards an anti-inflammatory state during airway inflammation and cutaneous wound repair. However, very little is known as to how E2 effects TAM function in tumors. In breast and ovarian cancer, tumor cell intrinsic E2/ER signaling has been linked to increased recruitment of TAMs in the tumor microenvironment. This study, on the other hand, highlighted a specific role for TAM intrinsic E2/ER signaling in promoting tumor growth in validated murine models of melanoma. It is demonstrated herein that inhibition of estrogen action in macrophages (depletion of ER) can recapitulate the systemic depletion of estrogen action on melanoma tumor growth. Therefore, it appears that most of the protumorigenic actions of E2 in the melanoma tumor microenvironment can be attributed to ER signaling in macrophages.

One of the most important findings in this study was that E2 polarized TAMs within the TME display the phenotypic features of M2-like immunosuppressive macrophages. This observation was confirmed by both flow cytometry analysis and by pseudotime analysis of gene expression from single cell RNA sequencing data, in which it was revealed that E2 leads to an initial accumulation of both inflammatory and patrolling monocytes. It then accelerates the polarization of inflammatory monocytes to M2 macrophages that express characteristic immune-suppressive markers (Cd163, Mrc1, Folr2, Retnla, and Gas6). However, the molecular mechanism(s) underlying this accelerated polarization of monocytes to macrophages remain to be determined.

The functional significance of an increased accumulation of immunosuppressive macrophages was highlighted by demonstrating that E2 treated TAMs blocked the cytotoxic activity of CD8⁺ T cells by preventing granzyme B expression and IFNγ release. Importantly, this activity was only manifested by macrophages residing in the tumor microenvironment and in BMDM cultured in TCM but not observed in BMDM cultured in NM. These results indicated that soluble factors secreted by tumor cells work in concert with E2 to promote TAM polarization that subsequently suppresses adaptive immunity. In line with that, changes in the expression of targets downstream of WNT5A/TCF4 signaling in tumor associated myeloid cells treated with E2 have been observed. Although functioning primarily as a positive regulator of the non-canonical WNT signaling pathway, WNT5A can in some contexts activate canonical WNT signaling through S-catenin to increase TCF/LEF transcriptional activity. Importantly, it has been demonstrated that tumor cell derived WNT5A can induce f-catenin activation in DCs leading to enhanced Indoleamine 2,3 dioxygenase (IDO) production, melanoma progression and M2 polarization. Since E2-mediated regulation of WNT5A targets in tumor associated myeloid cells was observed, it is speculated that tumor derived WNT5A may work in collaboration with E2 to skew macrophage polarization towards an immune-suppressive state and suppress T cell activity.

In contrast to CD8⁺ T cells, varying effects of E2 on CD4⁺ T cell activation and/or proliferation was observed when co-culturing with macrophages in vitro vs CD4⁺ T cells in E2 treated tumors in vivo. While in vito activated CD4⁺ T cells from naïve mice, co-cultured with myeloid cells isolated from E2 treated tumors ex vivo, demonstrate a decrease in proliferative and cytotoxic capabilities, there were no apparent differences in either proliferation or cytotoxicity of CD4⁺ T cells in placebo or E2 treated tumors. Apart from TAMs, the CD4⁺ T cells in the tumors are chronically exposed to cytokines and factors secreted by different cell types residing in the tumor which may account for lack of differences in their proliferative and cytotoxic states between placebo and E2, which is a possibility that is currently being explored.

ERα modulators are used as first-line treatment in ER+ breast cancer where tumor cell intrinsic actions of E2/ER axis facilitate tumor growth. The data presented herein demonstrated that in hormone-independent cancers (i.e., no direct effects of estrogens on cancer cells) like melanoma. ER antagonists/SERDs, such as fulvestrant, can efficiently suppress tumor growth by promoting anti-tumor immunity. The results of studies using tamoxifen in melanoma patients were equivocal, likely attributable to its inherent partial ER-agonistic activity. Fulvestrant is both a high affinity competitive antagonist and a receptor degrader allowing for a deep inhibition of ER action. Unfortunately, although an approved drug, its poor pharmaceutical properties has limited the clinical use of fulvestrant. Currently, there are twelve new orally bioavailable SERDs in clinical development, and there is an ongoing interest in evaluating the potential utility of these drugs as immune modulators. Moreover, useful cell/process selective ER inhibition can also be achieved using § elective Estrogen Receptor Modulators (SERMs) (i.e., bazedoxifene, lasofoxifene, and raloxifene), drugs whose relative agonist/antagonist properties differ depending on cell/tissue context. Thus, in addition to profiling new SERDs, these studies provide the rationale for testing different classes of SERDs and SERMs for their ability to reprogram macrophage function and increase tumor immunity in the setting of melanoma.

One of the most important findings of this study is that fulvestrant works in concert with ICBs to suppress melanoma tumor growth in both ICB sensitive and ICB unresponsive syngeneic models of melanoma. This can be attributed, at least in part, to the ability of fulvestrant to promote a pro immunogenic environment by elevating the M1 to M2 macrophage ratio and by increasing the number of intratumoral activated CD8⁺ T cells. This observation has significant clinical importance as although α-PD1 therapy is successful in some melanoma patients, the majority of treated patients do not respond to, or acquire resistance to, this intervention. It is believed that the findings in murine models of melanoma will translate to humans. This position is supported by the findings that a macrophage-derived, ER-downregulated, gene signature can predict survival in melanoma patients treated with ipilimumab and pembrolizumab/nivolumab. These findings highlighted the potential clinical utility of using a combination of ER modulators (SERDs or SERMs) with ICBs in melanoma patients who develop ICB resistance due to an increased accumulation of immune suppressive TAMs in tumors. Additionally, it was demonstrated that expression of the aromatase gene, correlates with enhanced expression of TAM markers such as CD68, CSF1R, CSF1, as well as a trend towards increased expression of PDCD1 in α-PD1 non-responders. This finding suggests that although patients who have higher levels of circulating estrogens are particularly vulnerable to develop resistance to α-PD1 therapy that intra-tumoral E2 production may also contribute to disease pathobiology. One of the major side effects of ICBs is the development of immune related adverse events (irAE), among which endocrine toxicities are most frequent. While the most common endocrinopathies related to ICB usage is associated with thyroid dysfunction, recent reports have also suggested a significant increase in risk of hypogonadism in ICB treated patients. Thus, the use of appropriate SERMs that demonstrate estrogenic action towards reproductive organs to ameliorate the inflammatory side effects of ICB, while at the same time promoting anti-tumor immunity, may have added clinical utility.

In conclusion, this study demonstrated that the E2/ER axis plays an important role in macrophage reprogramming within the melanoma TME and that specific targeting of the ER signaling axis in macrophages may improve the long-term survival of melanoma patients. While this study provided extensive evidence describing the role of ERα in modulating TAM polarization and suppression of adaptive immunity, the exact mechanism(s) by which E2 influences the immune suppressive activity of the TAM remain to be determined. Future studies addressing the possible mechanisms by which E2 influences TAM biology will be informative as to which of the existing SERMs or SERDs will be most useful for use in ICB regimens and/or help to define the characteristics of next generation ER-modulators optimized for their positive effects on tumor immunity. Additionally, while this study exclusively focuses on TAM intrinsic E2/ER signaling, melanoma cells express both nuclear ERs (ERα and ERβ) as well as GPER. While the functionality of these receptors in melanoma cells are yet to be studied in detail, the contribution of melanoma cell intrinsic E2/ER signaling to the tumor growth phenotype that has been observed cannot be completely ruled out. Studies using melanoma cells genetically depleted of ER may be informative as to the contribution of tumor cell intrinsic E2/ER signaling on melanoma biology.

Taken together, the results of these studies have provided the underlying rationale for a clinical study to explore the use of fulvestrant (and potentially other ER-modulators) as a means to increase the efficacy of immune checkpoint inhibitors.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure.

Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of treating cancer in a subject, the method comprising administering to the subject at least one estrogen receptor (ER) modulating drug and at least one additional therapy.

Clause 2. A method of treating cancer in a subject, the method comprising: administering to the subject at least one estrogen receptor (ER) modulating drug such that the effectiveness of an ICB therapy is increased relative to a control.

Clause 3. The method of clause 2, further comprising administering to the subject the ICB therapy.

Clause 4. The method of clause 2 or 3, wherein the ICB therapy is selected from anti-PD1, anti-CTLA4, anti-PDL1, and DMXAA, or a combination thereof.

Clause 5. The method of any one of clauses 2-4, wherein the effectiveness of the ICB therapy is increased by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control.

Clause 6. The method of any one of clauses 2-5, wherein the method further comprises administering to the subject at least one additional therapy.

Clause 7. The method of any one of clauses 1-6, wherein the at least one ER modulating drug is selected from a selective estrogen receptor modulator (SERM), a selective estrogen receptor degrader (SERD), an antiprogestin, an aromatase inhibitor, or a combination thereof.

Clause 8. The method of clause 7, wherein the SERM is selected from lasofoxifene, bazodoxifene, tamoxifen, raloxifene, clomiphene, ospemiphene, arzoxifene, toremifene, and H3B6545, or a combination thereof.

Clause 9. The method of any one of clauses 7-8, wherein the SERD is selected from fulvestrant, LSZ102, LY3484356, giredestrant, camizestrant, GDC0927, D-052, AC0682, AZD9496, SAR439859, RAD1901, G1T48, Zn-c5, ARV-471, and OP-1250, or a combination thereof.

Clause 10. The method of any one of clauses 7-9, wherein the antiprogestin is selected from mifepristone, asoprisnil, onapristone, and telapristone, or a combination thereof.

Clause 11. The method of any one of clauses 7-10, wherein the aromatase inhibitor is selected from letrozole, anastrozole, exemestane, vorozole, formestane, fadrozole, testolactone, aminoglutethimide, androstatrienedione, and 6-Oxo, or a combination thereof.

Clause 12. The method of any one of clauses 1-11, wherein the at least one additional therapy is selected from chemotherapy, immunotherapy, radiation therapy, hormone therapy, targeted drug therapy, cryoablation, and surgery, or a combination thereof.

Clause 13. The method of clause 12, wherein the chemotherapy is selected from an antimitotic agent, an alkylating agent, an antimetabolite, an antimicrotubule agent, a topoisomerase inhibitor, a cytotoxic agent, a cell cycle inhibitor, a growth factor inhibitor, a histone deacetylase (HDAC) inhibitor, and an inhibitor of a pathway that cross-talks with and activates ER transcriptional activity, or a combination thereof.

Clause 14. The method of clause 13, wherein the alkylating agent is selected from cisplatin, oxaliplatin, chlorambucil, procarbazine, and carmustine, or a combination thereof.

Clause 15. The method of clause 13 or 14, wherein the antimetabolite is selected from methotrexate, 5-fluorouracil, cytarabine, and gemcitabine, or a combination thereof.

Clause 16. The method of any one of clauses 13-15, wherein the antimicrotubule agent is selected from vinblastine and paclitaxel, or a combination thereof.

Clause 17. The method of any one of clauses 13-16, wherein the topoisomerase inhibitor is selected from etoposide and doxorubicin, or a combination thereof.

Clause 18. The method of any one of clauses 13-17, wherein the cytotoxic agent comprises bleomycin.

Clause 19. The method of any one of clauses 13-18, wherein the cell cycle inhibitor is selected from a cyclin-dependent kinase 4/6 (CDK4/6) inhibitor selected from palbociclib, abemaciclib, and ribociclib, or a combination thereof.

Clause 20. The method of any one of clauses 13-19, wherein the growth factor inhibitor is selected from a human epidermal growth factor receptor 2 (HER2) inhibitor such as trastuzumab, deruxtecan, sacitizumab, or ado-trastuzumab emtansine.

Clause 21. The method of any one of clauses 13-20, wherein the HDAC inhibitor is selected from vorinostat, romidepsin, chidamide, panobinostat, belinostat, Vvlproic acid, mocetinostat, abexinostat, entinostat, pracinostat, resminostat, givinostat, quisinostat, kevetrin, CUDC-101, AR-42, tefinostat, CHR-3996, 4SC202, CG200745, rocilinostat, and sulforaphane, or a combination thereof.

Clause 22. The method of clause 21, wherein the entinostat is not administered with an HER2 inhibitor.

Clause 23. The method of any one of clauses 13-22, wherein the inhibitor of a pathway that cross-talks with and activates ER transcriptional activity is selected from a phosphoinositide 3-kinase (PI3K) inhibitor, a heat shock protein 90 (HSP90) inhibitor, and a mammalian target of rapamycin (mTOR) inhibitor such as Everolimus.

Clause 24. The method of any one of clauses 12-23, wherein the immunotherapy is selected from a checkpoint inhibitor and denosumab, or a combination thereof.

Clause 25. The method of clause 24, wherein the checkpoint inhibitor is selected from anti-PD1, anti-CTLA4, anti-PDL1, and DMXAA, or a combination thereof.

Clause 26. The method of any one of clauses 12-25, wherein the targeted drug therapy is selected from vemurafenib, anti-EGFR targeted therapy, a serotonin-norepinephrine reuptake inhibitor (SNRI), a selective serotonin reuptake inhibitor (SSRI), and gabapentin, or a combination thereof.

Clause 27. The method of any one of clauses 1-27, wherein the at least one ER modulating drug is administered with anti-PD1, or with anti-CTLA4, or with anti-PD1 and anti-CTLA4.

Clause 28. The method of clause 27, wherein the method further comprises administering vemurafenib.

Clause 29. The method of any one of clauses 1-28, wherein the at least one ER modulating drug and the at least one additional therapy are administered simultaneously or sequentially.

Clause 30. The method of any one of clauses 28 and 29, wherein the at least one ER modulating drug and the at least one additional therapy and the vemurafenib are administered simultaneously or sequentially.

Clause 31. The method of any one of clauses 1-30, wherein the at least one ER modulating drug is administered to the subject once every day, once every 2 days, once every 3 days, once every 4 days, once every 5 days, once every 6 days, once every 7 days, once every week, once every 2 weeks, once every 3 weeks, once every 4 weeks, once every 5 weeks, once every 6 weeks, once every 7 weeks, once every 8 weeks, once every month, once every 2 months, once every 3 months, once every 4 months, once every 5 months, or once every 6 months.

Clause 32. The method of any one of clauses 1-31, wherein the at least one ER modulating drug is administered to the subject for 1 year, 2 years, 3 years, 4 years, 5 years, or more than 5 years.

Clause 33. The method of any one of clauses 1-32, wherein the at least one ER modulating drug is administered to the subject orally, intravenously, transdermally, or vaginally.

Clause 34. The method of any one of clauses 1-33, wherein the ER is ER-alpha or ER-beta.

Clause 35. The method of any one of clauses 1-34, wherein the cancer is selected from melanoma, colon cancer, breast cancer, and lung cancer.

Clause 36. The method of any one of clauses 1-35, wherein tumor-associated macrophage (TAM) polarization towards an immune suppressive phenotype is reduced, or wherein ER-alpha in myeloid cells is depleted, or wherein the Wnt 5A/TCF4 pathway is reduced, or wherein CD4+ T cell infiltration is not affected, or wherein an interferon pathway is reduced, or wherein CD8+ T cell proliferation is increased, or wherein CD8+ T cell migration is increased, or wherein CD8+ T cell cytotoxicity is increased, or wherein the ratio of M1/M2 macrophages is increased, or wherein tumor growth is decreased, or wherein tumor size is decreased, or wherein metastasis is reduced, or a combination thereof, in the subject.

Clause 37. A composition for treating cancer, the composition comprising at least one estrogen receptor (ER) modulating drug and at least one additional therapy.

Clause 38. The composition of clause 37, wherein the at least one ICB therapy is selected from anti-PD1, anti-CTLA4, anti-PDL1, and DMXAA, or a combination thereof.

Clause 39. A method of predicting response of a subject to ICB therapy, the method comprising: determining the level of expression in the subject of a gene selected from “Genes up-regulated upon E2 treatment” in TABLE 5 and/or “Genes down-regulated upon E2 treatment” in TABLE 5, wherein the level of expression of the gene selected from “Genes down-regulated upon E2 treatment” is increased relative to a control, and/or wherein the level of expression of the gene selected from “Genes up-regulated upon E2 treatment” is decreased relative to a control; and identifying the subject as responsive to ICB therapy.

Clause 40. The method of clause 39, further comprising administering to the subject at least one ICB therapy.

Clause 41. The method of clause 40, wherein the at least one ICB therapy is selected from anti-PD1, anti-CTLA4, anti-PDL1, and DMXAA, or a combination thereof.

SEQUENCES

SEQ ID NO: 1
CCTGCACTGAATCAAGAGGTTGC
SEQ ID NO: 2
CCATCAGAAGGACTTGCTGGCT
SEQ ID NO: 3
GCTACCAGATGGAAACTGTGCG
SEQ ID NO: 4
GCCTCTGTAATGTTGGTGAGATC
SEQ ID NO: 5
GCAACTCGACTATGGCATCTACG
SEQ ID NO: 6
CGGAATGAGGTCCACATCACTG
SEQ ID NO: 7
GCCTCTCATTGTCACCGAAGAAC
SEQ ID NO: 8
TGGCTGACGTTGGAGATCACCA
SEQ ID NO: 9
GCAGTGAGCATCGAAGAGTACC
SEQ ID NO: 10
GGCAAAAGCATCTGGAGTTCCAG
SEQ ID NO: 11
GAAGGAACACGGCTGTCAGCAA
SEQ ID NO: 12
GATGAGCAGGTTGCTGTTCTGG
SEQ ID NO: 13
GAGCCTACTTTGTACGTGGCGA
SEQ ID NO: 14
GCTCTTTCCAGACTATGTAGGAG
SEQ ID NO: 15
CATCAGTGTGCCTGTACCAGAG
SEQ ID NO: 16
GAGGAATCACGTAGCACTTGTCC
SEQ ID NO: 17
CTGCTGTAACGATGAAGCCCTG
SEQ ID NO: 18
GCTGTAGGAAGCTCATCTCTCC
SEQ ID NO: 19
TCTGTGCCAGACACGATTGACG
SEQ ID NO: 20
GAGGCAGAGTTCAGAGCCAAGT
SEQ ID NO: 21
CCTCTCATCACCTACAGCAACG
SEQ ID NO: 22
CTGGAGACAGTGGGTAATACGG
SEQ ID NO: 23
AGGTGCCCTACTACCTGGAG
SEQ ID NO: 24
GTCTCTCTCGGCCATTCTGG
SEQ ID NO: 25
CATCCATCCATCCATCCA
SEQ ID NO: 26
TTCAGGGTCATGGAGACAGT
SEQ ID NO: 27
AGGTCTACCCGCCATACCTT
SEQ ID NO: 28
CGCCATAGTGACAGCCAGAT
SEQ ID NO: 29
AGATTCGGGATATGCTGTTGGC
SEQ ID NO: 30
TCGGGTCCTAGACCAGTGTTC
SEQ ID NO: 31
AUAUUCAGAAUAGAUCAUGGG
SEQ ID NO: 32
UGUGCUUCAACAUUCUCCCTC
SEQ ID NO: 33
UGCUUAAUCACAAGAGGGCTT

1. A method of treating cancer in a subject, the method comprising administering to the subject at least one estrogen receptor (ER) modulating drug and at least one additional therapy.

2. A method of treating cancer in a subject, the method comprising administering to the subject at least one estrogen receptor (ER) modulating drug such that the effectiveness of an ICB therapy is increased relative to a control.

3. The method of claim 2, further comprising administering to the subject the ICB therapy.

4. The method of claim 2 or 3, wherein the ICB therapy is selected from anti-PD1, anti-CTLA4, anti-PDL1, and DMXAA, or a combination thereof.

5. The method of any one of claims 2-4, wherein the effectiveness of the ICB therapy is increased by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control.

6. The method of any one of claims 2-5, wherein the method further comprises administering to the subject at least one additional therapy.

7. The method of any one of claims 1-6, wherein the at least one ER modulating drug is selected from a selective estrogen receptor modulator (SERM), a selective estrogen receptor degrader (SERD), an antiprogestin, an aromatase inhibitor, or a combination thereof.

8. The method of claim 7, wherein the SERM is selected from lasofoxifene, bazodoxifene, tamoxifen, raloxifene, clomiphene, ospemiphene, arzoxifene, toremifene, and H3B6545, or a combination thereof.

9. The method of any one of claims 7-8, wherein the SERD is selected from fulvestrant, LSZ102, LY3484356, giredestrant, camizestrant, GDC0927, D-052, AC0682, AZD9496, SAR439859, RAD1901, G1T48, Zn-c5, ARV-471, and OP-1250, or a combination thereof.

10. The method of any one of claims 7-9, wherein the antiprogestin is selected from mifepristone, asoprisnil, onapristone, and telapristone, or a combination thereof.

11. The method of any one of claims 7-10, wherein the aromatase inhibitor is selected from letrozole, anastrozole, exemestane, vorozole, formestane, fadrozole, testolactone, aminoglutethimide, androstatrienedione, and 6-Oxo, or a combination thereof.

12. The method of any one of claims 1-11, wherein the at least one additional therapy is selected from chemotherapy, immunotherapy, radiation therapy, hormone therapy, targeted drug therapy, cryoablation, and surgery, or a combination thereof.

13. The method of claim 12, wherein the chemotherapy is selected from an antimitotic agent, an alkylating agent, an antimetabolite, an antimicrotubule agent, a topoisomerase inhibitor, a cytotoxic agent, a cell cycle inhibitor, a growth factor inhibitor, a histone deacetylase (HDAC) inhibitor, and an inhibitor of a pathway that cross-talks with and activates ER transcriptional activity, or a combination thereof.

14. The method of claim 13, wherein the alkylating agent is selected from cisplatin, oxaliplatin, chlorambucil, procarbazine, and carmustine, or a combination thereof.

15. The method of claim 13 or 14, wherein the antimetabolite is selected from methotrexate, 5-fluorouracil, cytarabine, and gemcitabine, or a combination thereof.

16. The method of any one of claims 13-15, wherein the antimicrotubule agent is selected from vinblastine and paclitaxel, or a combination thereof.

17. The method of any one of claims 13-16, wherein the topoisomerase inhibitor is selected from etoposide and doxorubicin, or a combination thereof.

18. The method of any one of claims 13-17, wherein the cytotoxic agent comprises bleomycin.

19. The method of any one of claims 13-18, wherein the cell cycle inhibitor is selected from a cyclin-dependent kinase 4/6 (CDK4/6) inhibitor selected from palbociclib, abemaciclib, and ribociclib, or a combination thereof.

20. The method of any one of claims 13-19, wherein the growth factor inhibitor is selected from a human epidermal growth factor receptor 2 (HER2) inhibitor such as trastuzumab, deruxtecan, sacitizumab, or ado-trastuzumab emtansine.

21. The method of any one of claims 13-20, wherein the HDAC inhibitor is selected from vorinostat, romidepsin, chidamide, panobinostat, belinostat, Vvlproic acid, mocetinostat, abexinostat, entinostat, pracinostat, resminostat, givinostat, quisinostat, kevetrin, CUDC-101, AR-42, tefinostat, CHR-3996, 4SC202, CG200745, rocilinostat, and sulforaphane, or a combination thereof.

22. The method of claim 21, wherein the entinostat is not administered with an HER2 inhibitor.

23. The method of any one of claims 13-22, wherein the inhibitor of a pathway that cross-talks with and activates ER transcriptional activity is selected from a phosphoinositide 3-kinase (PI3K) inhibitor, a heat shock protein 90 (HSP90) inhibitor, and a mammalian target of rapamycin (mTOR) inhibitor such as Everolimus.

24. The method of any one of claims 12-23, wherein the immunotherapy is selected from a checkpoint inhibitor and denosumab, or a combination thereof.

25. The method of claim 24, wherein the checkpoint inhibitor is selected from anti-PD1, anti-CTLA4, anti-PDL1, and DMXAA, or a combination thereof.

26. The method of any one of claims 12-25, wherein the targeted drug therapy is selected from vemurafenib, anti-EGFR targeted therapy, a serotonin-norepinephrine reuptake inhibitor (SNRI), a selective serotonin reuptake inhibitor (SSRI), and gabapentin, or a combination thereof.

27. The method of any one of claims 1-27, wherein the at least one ER modulating drug is administered with anti-PD1, or with anti-CTLA4, or with anti-PD1 and anti-CTLA4.

28. The method of claim 27, wherein the method further comprises administering vemurafenib.

29. The method of any one of claims 1-28, wherein the at least one ER modulating drug and the at least one additional therapy are administered simultaneously or sequentially.

30. The method of any one of claims 28 and 29, wherein the at least one ER modulating drug and the at least one additional therapy and the vemurafenib are administered simultaneously or sequentially.

31. The method of any one of claims 1-30, wherein the at least one ER modulating drug is administered to the subject once every day, once every 2 days, once every 3 days, once every 4 days, once every 5 days, once every 6 days, once every 7 days, once every week, once every 2 weeks, once every 3 weeks, once every 4 weeks, once every 5 weeks, once every 6 weeks, once every 7 weeks, once every 8 weeks, once every month, once every 2 months, once every 3 months, once every 4 months, once every 5 months, or once every 6 months.

32. The method of any one of claims 1-31, wherein the at least one ER modulating drug is administered to the subject for 1 year, 2 years, 3 years, 4 years, 5 years, or more than 5 years.

33. The method of any one of claims 1-32, wherein the at least one ER modulating drug is administered to the subject orally, intravenously, transdermally, or vaginally.

34. The method of any one of claims 1-33, wherein the ER is ER-alpha or ER-beta.

35. The method of any one of claims 1-34, wherein the cancer is selected from melanoma, colon cancer, breast cancer, and lung cancer.

36. The method of any one of claims 1-35, wherein tumor-associated macrophage (TAM) polarization towards an immune suppressive phenotype is reduced, or wherein ER-alpha in myeloid cells is depleted, or wherein the Wnt 5A/TCF4 pathway is reduced, or wherein CD4+ T cell infiltration is not affected, or wherein an interferon pathway is reduced, or wherein CD8+ T cell proliferation is increased, or wherein CD8+ T cell migration is increased, or wherein CD8+ T cell cytotoxicity is increased, or wherein the ratio of M1/M2 macrophages is increased, or wherein tumor growth is decreased, or wherein tumor size is decreased, or wherein metastasis is reduced, or a combination thereof, in the subject.

37. A composition for treating cancer, the composition comprising at least one estrogen receptor (ER) modulating drug and at least one additional therapy.

38. The composition of claim 37, wherein the at least one ICB therapy is selected from anti-PD1, anti-CTLA4, anti-PDL1, and DMXAA, or a combination thereof.

39. A method of predicting response of a subject to ICB therapy, the method comprising:
determining the level of expression in the subject of a gene selected from “Genes up-regulated upon E2 treatment” in TABLE 5 and/or “Genes down-regulated upon E2 treatment” in TABLE 5,
wherein the level of expression of the gene selected from “Genes down-regulated upon E2 treatment” is increased relative to a control, and/or
wherein the level of expression of the gene selected from “Genes up-regulated upon E2 treatment” is decreased relative to a control; and
identifying the subject as responsive to ICB therapy.

40. The method of claim 39, further comprising administering to the subject at least one ICB therapy.

41. The method of claim 40, wherein the at least one ICB therapy is selected from anti-PD1, anti-CTLA4, anti-PDL1, and DMXAA, or a combination thereof.
